(19) United States
(12) Patent Application Publication
(10) Pub. No.: US 2024/0327477 A1
(43) Pub. Date: Oct. 3, 2024

(54) COMPOSITIONS AND METHODS FOR THE TREATMENT OF TDP-43 PROTEINOPATHIES

(71) Applicant: SOLA BIOSCIENCES LLC, Natick, MA (US)

(72) Inventors: Akinori Hishiya, Belmont, MA (US); Keizo Koya, Chestnut Hill, MA (US)

(21) Appl. No.: 17/997,142

(22) PCT Filed: Apr. 27, 2021
(86) PCT No.: PCT/US21/29289

§ 371 Date: May 12, 2023

Related U.S. Application Data
(60) Provisional application No. 63/035,437, filed on Jun. 5, 2020.
(60) Provisional application No. 63/016,707, filed on Apr. 28, 2020.

(51) Int. Cl. C07K 14/47; A61K 38/00; A61P 25/28; C12N 15/86

(52) U.S. Cl. CPC C07K 14/4702; A61P 25/28; C12N 15/86; A61K 38/00; C07K 2319/01; C12N 2750/14143

(57) ABSTRACT

A novel class of fusion proteins to recruit a cell's innate chaperone mechanism, specifically the Hsp70-mediated system, to specifically reduce TDP-43-mediated protein aggregation and associated proteopathies is disclosed.

### CROSS REFERENCE TO RELATED APPLICATION

This application claims priority under 35 U.S.C. § 119 (e) to the U.S. Provisional Patent Application 63/016,707 filed Apr. 28, 2020, and also to U.S. Provisional Patent Application 63/035,437 filed Jun. 5, 2020. The entire contents of the aforementioned applications are hereby incorporated by reference in their entireties.

### BACKGROUND

All proteins expressed within a cell need to correctly fold into their intended structures in order to function properly. A growing number of diseases and disorders are shown to be associated with inappropriate folding of proteins and/or inappropriate deposition and aggregation of proteins and lipoproteins as well as infectious proteinaceous substances. Also known as a conformational disease or proteopathy, examples of diseases caused by misfolding include Alzheimer's disease (AD), amyotrophic lateral sclerosis (ALS), and frontotemporal lobar dementia (FTLD). The mutant protein aggregates in cells causing typical cytotoxic cellular inclusion bodies.

A wide variety of neurodegenerative diseases are characterized pathologically by the accumulation of intracellular or extracellular protein aggregates composed of amyloid fibrils (Forman et al., (2004) Nat Med. 10:1055-1063). For example, the pathology of Alzheimer's disease (AD) is defined by senile plaques and neurofibrillary tangles composed of β-amyloid and microtubule-associated protein tau, respectively, and Lewy bodies composed of α-synuclein are the disease-defining lesions of Parkinson's disease. Until recently, the neuropathology of both frontotemporal lobar degeneration with ubiquitin inclusions (FTLD-U) (Kumar-Singh & Van (2007) Brain Pathol., 17:104-114), the most common phenotype associated with the FTLD syndrome, and amyotrophic lateral sclerosis (ALS) (Xiao et al., (2006) Biochim Biophys Acta, 1762:1001-1012) were defined by non-amyloidogenic ubiquitinated inclusions (UBI).

FTLD, the second most common form of presenile dementia, refers to a heterogeneous group of neurodegenerative disorders that have in common behavioral and/or language dysfunction (Kumar-Singh & Van, ibid). Some affected individuals manifest a movement disorder such as parkinsonism or motor neuron disease (MND). While the designation FTLD reflects the prominent frontal and temporal lobe degeneration, multiple neuropathological abnormalities are identified in these patients (Cairns et al., (2007) Acta Neuropathol., 1145:5-22). Two broad pathological subdivisions of FTLD are recognized: brains with tau-positive inclusions (i.e., tauopathies) and brains with UBI that are not detected with antibodies to tau, α-synuclein, and β-amyloid (i.e., FTLD-U). Up to 40% of FTLD show a familial pattern of inheritance with three different genetic abnormalities associated with FTLD-U pathology including mutations in progranulin (PGRN) and valosin-containing protein (VCP) as well as linkage to a novel locus on chromosome 9p.

Amyotrophic lateral sclerosis (ALS), also known as motor neuron disease or Lou Gehrig's disease, is a neurodegenerative disease characterized by progressive degeneration of both upper and lower motor neurons in the brainstem and spinal cord, leading to progressive muscle wasting and weakness (Al-Chalabi et al., (2016) Amyotrophic lateral sclerosis., 15 (11): 1182-1194; Robberecht & Philips, (2013) Nat Rev Neurosci., 14 (4): 248-264; Talbot et al., (2018) Nucleic Acids Res., 37 (8): e64). ALS has a median prevalence about 5.4 cases per 100,000 persons for a median age of onset at 54-67 years old, with men at a slightly higher risk compared to women (Chio et al., (2009) Amyotroph Lateral Scler., 10 (5-6): 310-323; Chio et al., (2013) Neuroepidemiology, 41 (2): 118-130; McCombe & Henderson, (2010) Gend Med., 7 (6): 557-570). ALS is a devastating neurodegenerative disease without any effective treatment, and patients usually die within 2-4 years from disease onset, primarily due to respiratory failure and swallowing problems (Chio et al., (2009) Amyotroph Lateral Scler., 10 (5-6): 310-323; del Aguila et al., (2003) Neurology, 60 (5): 813-819; Tabata et al., (2009) Nucleic Acids Res., 7 (8): e64).

The majority of ALS cases are sporadic with unknown cause (sALS) while only ˜10% cases involve Mendelian pattern of inheritance of familial gene mutations known as familial ALS (fALS) (Renton et al., (2014) Nat Neurosci., 17 (1): 17-23; Taylor et al., (2016) Nature, 539 (7628): 197-206; Turner et al., (2017) J Neurol Neurosurg Psychiatry, 88 (12): 1042-1044). To date, up to 30 genes are described as monogenic causes of ALS, with the most frequent being C9orf72, SOD1, FUS, and TARDBP/TDP43 (Chia et al., (2018) Lancet Neurol., 17 (1): 94-102; Nicolas et al., (2018) Neuron, 97 (6): 1268-1283; Volk et al., (2018) Med Genet., 30 (2): 252-258).

The variability in penetrance as well as identification of gene mutations in a large number of genes in ALS suggests that multiple factors such as multigenic components and environmental factors can underlie the disease susceptibility. In fact, it has been suggested that a combination of cellular pathways such as protein misfolding/aggregation (Ross & Poirier, (2004) Nat Med., 10 Suppl: S10-17), glutamate-mediated excitotoxicity (Blasco et al., (2014) Curr Med Chem., 21 (31): 3551-3575), mitochondrial dynamic abnormalities (Cappello & Francolini, (2017) Int J Mol Sci., 18 (10); Delic et al., (2018) J Neurosci Res., 96 (8): 1353-1366; Onesto et al., (2016) Acta Neuropathol Commun., 4 (1): 47), and contributors to oxidative stress (Anand et al., (2013) Oxid Med Cell Longev., 2013:635831; Sharma et al., (2016) Neurochem Res., 41 (5): 965-984) can lead to neurotoxic outcomes in ALS.

The transactive response (TAR)-DNA binding Protein with a molecular weight of 43 KDa (TDP-43) was identified as the major disease protein in the UBI of FTLD-U and ALS (Neumann et al., (2006) Science 314:130-133). The identification of TDP-43 pathology in both of these disorders provided a mechanistic link for the following: 1) a large proportion of ALS patients manifest a range of behavioral and cognitive changes that lie on the spectrum of FTLD (Murphy et al., (2007) Arch. Neurol., 64:330-334); 2) MND is commonly observed in FTLD-U patients (McKhann et al., (2001) Arch. Neurol., 58:1803-1809); 3) there is significant overlap in the ubiquitin pathology observed in ALS and FTLD-U (Mackenzie & Feldman (2005) J. Neuropathol. Exp. Neurol. 64:730-739); and 4) identification of genetic loci and mutations in specific genes in families with co-segregation of both ALS and FTLD (Talbot & Ansorge (2006) Hum. Mol. Genet., 15: R183-R187). TDP-43 has also been shown to be a histopathological marker of multiple other neurodegenerative diseases, including Alzheimer's disease (Amador-Ortiz et al., (2007), Ann Neurol., 61:435-45), Parkinson's disease (Lin and Dickson, (2008) Acta Neuropathol., 116:205-13), and Huntington's disease (Schwab et al., (2008), J Neuropathol Exp Neurol., 67:1159-65), hippocampal sclerosis (Amador-Ortiz et al., (2007), ibid), and dementia with Lewy's bodies (Lin and Dickson, (2008), ibid); reviewed in (Lagier-Tourenne et al., (2010), Human Molecular Genetics, 19: R46-R64).

Therefore, a need exists for the development of new therapeutic modalities optimized to target specific antigens, proteins, glycoproteins or lipoproteins, particularly against diseases with pathologies based on protein misfolding and aggregation, as well as in cases of heterogeneous aggregates.

The heat shock 70 kDa proteins (referred to herein as “Hsp70s”) constitute a ubiquitous class of chaperone proteins in the cells of a wide variety of species (Tavaria et al., (1996) Cell Stress Chaperones 1, 23-28). Hsp70 requires assistant proteins called co-chaperone proteins, such as J domain proteins and nucleotide exchange factors (NEFs) (Hartl et al., (2009) Nat Struct Mol Biol 16, 574-581), in order to function. In the current model of Hsp70 chaperone machinery for folding proteins, Hsp70 cycles between ATP- and ADP-bound states, and a J domain protein binds to another protein in need of folding or refolding (referred to as a “client protein”), interacting with the ATP-bound form of Hsp70 (Hsp70-ATP) (Young (2010) Biochem Cell Biol 88, 291-300; Mayer, (2010) Mol Cell 39, 321-331). Binding of the J domain protein-client complex to Hsp70-ATP stimulates ATP hydrolysis, which causes a conformational change in the Hsp70 protein, closing a helical lid and, thereby, stabilizing the interaction between the client protein with Hsp70-ADP, as well as eliciting the release of the J domain protein that is then free to bind to another client protein.

Therefore, according to this model, J domain proteins play a critical role within the Hsp70 machinery by acting as a bridge, and facilitating the capture and submission of a wide variety of client proteins into the Hsp70 machinery to promote folding or refolding into the proper conformation (Kampinga & Craig (2010) Nat Rev Mol Cell Biol 11, 579-592). The J domain family is widely conserved in species ranging from prokaryotes (DnaJ protein) to eukaryotes (Hsp40 protein family). The J domain (about 60-80 aa) is composed of four helices: I, II, III, and IV. Helices II and III are connected via a flexible loop containing an “HPD motif”, which is highly conserved across J domains and thought to be critical for activity (Tsai & Douglas, (1996) J Biol Chem 271, 9347-9354). Mutations within the HPD sequence has been found to abolish J domain function.

Given the context provided above for proteopathies such as ALS, it seems clear that reducing the level of misfolded proteins could serve as a means to treat, prevent or otherwise ameliorate the symptoms of these devastating disorders and that, recruitment of a cell's innate ability to repair protein misfolding would be a logical choice to pursue.

### Summary of the Invention

The inventors have developed a novel class of fusion proteins to recruit a cell's innate chaperone mechanism, specifically the Hsp70-mediated system, to specifically reduce TDP-43-mediated protein aggregation. Unlike in previous studies by the inventors using fusion proteins comprising fragments of a Hsp40 protein (also called J proteins), a co-chaperone that interacts with Hsp70, to enhance protein secretion and expression, the present study employs J domain-containing fusion proteins for the purpose of reducing protein aggregation and cytotoxicity caused by aggregation of mutant TDP-43 proteins. In this context, the inventors have made the surprising discovery that the elements of J domain required for function is quite distinct from use of J domains in enhancing protein expression and secretion, demonstrating a distinct mechanism for the mode of action of the present fusion proteins. The fusion proteins described herein comprise a J domain and a domain that has affinity for TDP-43. The presence of the TDP-43-binding domain within the fusion protein results in specific reduction in aggregation of mutant TDP-43 proteins.

E1. Therefore, in a first aspect, disclosed herein is an isolated fusion protein comprising a J domain of a J protein and a TDP-43-binding domain.

E2. The fusion protein of E1, wherein the J domain of a J protein is of eukaryotic origin.

E3. The fusion protein of any one of E1-E2, wherein the J domain of a J protein is of human origin.

E4. The fusion protein of any one of E1-E3, wherein the J domain of a J protein is cytosolically localized.

E5. The fusion protein of any one of E1-E4, wherein the J domain of a J protein is selected from the group consisting of SEQ ID Nos: 1-50.

E6. The fusion protein of any one of E1-E5, wherein the J domain comprises the sequence selected from the group consisting of SEQ ID NOs: 1, 5, 6, 10, 16, 24, 25, 31 and 49.

E7. The fusion protein of any one of E1-E6, wherein the J domain comprises the sequence of SEQ ID NO: 5.

E8. The fusion protein of any one of E1-E6, wherein the J domain comprises the sequence of SEQ ID NO: 10.

E9. The fusion protein of any one of E1-E6, wherein the J domain comprises the sequence of SEQ ID NO: 16.

E10. The fusion protein of any one of E1-E6, wherein the J domain comprises the sequence of SEQ ID NO: 25.

E11. The fusion protein of any one of E1-E6, wherein the J domain comprises the sequence of SEQ ID NO: 31.

E12. The fusion protein of any one of E1-E11, wherein the TDP-43-binding domain has a K_{D} for TDP-43 (for example, using a reporter construct comprising the C-terminal 207 amino acids of TDP-43) of 1 UM or less, for example, 300 nM or less, 100 nM or less, 30 nM or less, 10 nM or less, for example when measured using an ELISA assay.

E13. The fusion protein of any one of E1-E12, wherein the TDP-43-binding domain comprises the sequence selected from the group consisting of SEQ ID NOs: 51-55.

E14. The fusion protein of any one of E1-E13, wherein the TDP-43-binding domain comprises the sequence of SEQ ID NO:51-53.

E15. The fusion protein of any one of E1-E13, wherein the TDP-43-binding domain comprises the sequence of SEQ ID NO:51.

E16. The fusion protein of any one of E1-E13, wherein the TDP-43-binding domain comprises the sequence of SEQ ID NO:53.

E17. The fusion protein of any one of E1-E16, comprising a plurality of TDP-43-binding domains.

E18. The fusion protein of any one of E1-E17, consisting of two TDP-43-binding domains.

E19. The fusion protein of any one of E1-E18, consisting of three TDP-43-binding domains.

E20. The fusion protein of any one of E1-E19, comprising one of the following constructs:
a. DNAJ-X-T,
b. DNAJ-X-T-X-T,
c. DNAJ-X-T-X-T-X-T,
d. T-X-DNAJ,
e. T-X-T-X-DNAJ,
f. T-X-T-X-T-X-DNAJ,
g. T-X-DNAJ-X-T,
h. T-X-DNAJ-X-T-X-T,
i. TDNAJ-X-TTTTTDNAJ-X-T,
j. T-X-T-X-DNAJ-X-TT,
k. TTDNAJ-X-T-X-TTTTTDNAJ-X-T,
l. T-X-T-X-DNAJ-X-T-X-T-X-T,
m. T-X-T-X-T-X-DNAJ-X-T,
n. T-X-T-X-T-X-DNAJ-X-T-X-T,
o. T-X-T-X-T-X-DNAJ-X-T-X-T-X-T,
p. DnaJ-X-DnaJ-X-T-X-T,
q. T-X-DnaJ-X-DnaJ,
r. T-X-T-X-DnaJ-X-DnaJ, and
s. T-X-TDnaJ-X-TDnaJ-X-TTTT
wherein,
T is a TDP-43-binding domain,
DNAJ is a J domain of a J protein, and
X is an optional linker.

E21. The fusion protein of any one of E1-E20, wherein the fusion protein comprises the J domain sequence of SEQ ID NO: 5 and the TDP-43-binding domain sequence of SEQ ID NO: 51.

E22. The fusion protein of any one of E1-E21, wherein the fusion protein comprises the J domain sequence of SEQ ID NO: 5 and two copies of the TDP-43-binding domain sequence of SEQ ID NO: 53.

E23. The fusion protein of any one of E1-E22, wherein the fusion protein comprises the sequence selected from the group consisting of SEQ ID NOs: 80-85 and 89-97.

E24. The fusion protein of any one of E1-E23, wherein the fusion protein comprises the sequence selected from the group consisting of SEQ ID NOs: 80, 82-85, 89-90 and 92-97.

E25. The fusion protein of any one of E1-E23, wherein the fusion protein comprises the sequence of SEQ ID NO: 80.

E26. The fusion protein of any one of E1-E23, wherein the fusion protein comprises the sequence of SEQ ID NO: 90.

E27. The fusion protein of any one of E1-E23, wherein the fusion protein comprises the sequence of SEQ ID NO: 92.

E28. The fusion protein of any one of E1-E23, wherein the fusion protein comprises the sequence of SEQ ID NO: 94.

E29. The fusion protein of any one of E1-E23, wherein the fusion protein comprises the sequence of SEQ ID NO: 95.

E30. The fusion protein of any one of E1-E23, wherein the fusion protein comprises the sequence of SEQ ID NO: 96.

E31. The fusion protein of any one of E1-E30, further comprising a targeting reagent.

E32. The fusion protein of any one of E1-E31, further comprising an epitope.

E33. The fusion protein of E32, wherein the epitope is a polypeptide selected from the group consisting of SEQ ID NOs: 67-73.

E34. The fusion protein of any one of E1-E33, further comprising a cell-penetrating agent.

E35. The fusion protein of E34, wherein the cell-penetrating agent is selected from the group consisting of SEQ ID NOs: 74-77.

E36. The fusion protein of any one of E1-E35, further comprising a signal sequence.

E37. The fusion protein of E36, wherein the signal sequence comprises the peptide sequence selected from the group consisting of SEQ ID NOs: 98-100.

E38. The fusion protein of any one of E1-E37, which is capable of reducing aggregation of TDP-43 proteins in a cell.

E39. The fusion protein of any one of E1-E38, which is capable of reducing TDP-43-mediated cytotoxicity.

E40. A nucleic acid sequence encoding the fusion protein of any one of E1-E39.

E41. The nucleic acid sequence of E40, wherein said nucleic acid is DNA.

E42. The nucleic acid sequence of any one of E40, wherein said nucleic acid is RNA.

E43. The nucleic acid sequence of any one of E40-E42, wherein said nucleic acid comprises at least one modified nucleic acid.

E44. The nucleic acid sequence of any one of E40-E43, further comprising a promoter region, 5′ UTR, 3′ UTR such as poly (A) signal.

E45. The nucleic acid sequence of E44, wherein the promoter region comprises a sequence selected from the group consisting of a CMV enhancer sequence, a CMV promoter, a CBA promoter, UBC promoter, GUSB promoter, NSE promoter, Synapsin promoter, MeCP2 promoter and GFAP promoter.

E46. A vector comprising the nucleic acid sequence of any one of E40-E45.

E47. The vector of E46, wherein the vector is selected from the group consisting of adeno-associated virus (AAV), adenovirus, lentivirus, retrovirus, herpesvirus, poxvirus (vaccinia or myxoma), paramyxovirus (measles, RSV or Newcastle disease virus), baculovirus, reovirus, alphavirus, and flavivirus.

E48. The vector of E46 or E47, wherein the vector is an AAV.

E49. A virus particle comprising a capsid and the vector of any one of E46-E48.

E50. The virus particle of E49, wherein the capsid is selected from the group consisting of AAV1, AAV2, AAV3, AAV4, AAV5, AAV6, AAV7, AAV8, AAV9, AAV10 AAV11, AAV12, pseudotyped AAV, a rhesus-derived AAV, AAVrh8, AAVrh10 and AAV-DJan AAV capsid mutant, an AAV hybrid serotype, an organ-tropic AAV, a cardiotropic AAV, and a cardiotropic AAVM41 mutant.

E51. The virus particle of E49 or E50, wherein the capsid is selected from the group consisting of AAV2, AAV5, AAV8, AAV9 and AAVrh10.

E52. The virus particle of any one of E49-E51, wherein the capsid is AAV2.

E53. The virus particle of any one of E49-E51, wherein the capsid is AAV5.

E54. The virus particle of any one of E49-E51, wherein the capsid is AAV8.

E55. The virus particle of any one of E49-E51, wherein the capsid is AAV9.

E56. The virus particle of any one of E49-E51, wherein the capsid is AAV rh10.

E57. A pharmaceutical composition comprising an agent selected from the group consisting of the fusion protein of any one of E1-E39, a cell expressing the fusion protein of E1-E39, the nucleic acid of any one of E40-E45, the vector of any one of E46-E48, the virus particle of any one of E49-E56, and a pharmaceutically acceptable carrier or excipient.

E58. A method of reducing toxicity of a TDP-43 protein in a cell, comprising contacting said cell with an effective amount of one or more agents selected from the group consisting of the fusion protein of any one of E1-E39, a cell expressing the fusion protein of E1-E39, the nucleic acid of any one of E40-E45, the vector of any one of E46-E48, the virus particle of any one of E49-E56, and the pharmaceutically composition of E57.

E59. The method of E58, wherein the cell is in a subject.

E60. The method of any one of E58-E59, wherein the subject is a human.

E61. The method of any one of E58-E60, wherein the cell is a cell of the central nervous system and peripheral nervous system.

E62. The method of any one of E58-E61, wherein subject is identified as having a TDP-43 disease.

E63. The method of E62, wherein the TDP-43 disease is selected from the group consisting of ALS, FTD, Parkinson's disease, Huntington's disease, Alzheimer's disease, hippocampal sclerosis, dementia with Lewy's bodies, and limbic predominant age-related TDP-43 encephalopathy.

E64. The method of E62 or E63, wherein the TDP-43 disease is ALS.

E65. The method of any one of E58-E64, wherein there is a reduction in the amount of aggregated TDP-43 protein in the cell when compared with a control cell.

E66. A method of treating, preventing, or delaying the progression of a TDP-43 disease in a subject in need thereof, the method comprising administering an effective amount of one or more agents selected from the group consisting of the fusion protein of any one of E1-E39, a cell expressing the fusion protein of E1-E39, the nucleic acid of any one of E40-E45, the vector of any one of E46-E48, the virus particle of any one of E49-E56, and the pharmaceutically composition of E57 . . .

E67. The method of E66, wherein the TDP-43 disease is selected from the group consisting of ALS, FTD, Parkinson's disease, Huntington's disease, Alzheimer's disease, hippocampal sclerosis, dementia with Lewy's bodies, and limbic predominant age-related TDP-43 encephalopathy.

E68. The method of E67, wherein the TDP-43 disease is ALS.

E69. Use of one or more of the fusion protein of any one of E1-E39, a cell expressing the fusion protein of E1-E39, the nucleic acid of any one of E40-E45, the vector of any one of E46-E48, the virus particle of any one of E49-E56, and the pharmaceutically composition of E57, in the preparation of a medicament useful for the prevention or delay of progression of a TDP-43 disease in a subject.

E70. The Use of E69, wherein the TDP-43 disease is selected from the group consisting of ALS, FTD, Parkinson's disease, Huntington's disease, Alzheimer's disease, hippocampal sclerosis, dementia with Lewy's bodies, and limbic predominant age-related TDP-43 encephalopathy.

E71. The use of E69 or E70, wherein the TDP-43 disease is ALS.

### DESCRIPTION OF THE FIGURES

FIG. 1A shows a Clustal Omega sequence alignment of representative human J domain sequences. The highly conserved HPD domain is shown in the highlighted box.

FIG. 1B shows a Clustal Omega sequence alignment of representative human J domain sequences.

FIG. 2 shows some illustrative fusion protein constructs comprising a J domain and TDP-43-binding domains.

FIG. 3 shows visualization of aggregation of TDP43-GFP fusion constructs in cells as measured by fluorescence microscopy, and the effect of J domain fusion proteins in reducing the aggregation. Fluorescence microscopy of cells transfected with GFP reporter constructs containing either full-length TDP-43 (GFP-TDP43FL; panels 1-4) or the C-terminal fragment of TDP-43 (GFP-TDPCTF; panels 5-8) further containing scFv controls (panels 2 and 6), DnaJB1-scFv (3B12A) fusion protein (panels 3 and 7), and DnaJB1-scFv (3B12A) fusion protein containing a P33Q mutation in the conserved HPD domain (panels 4 and 8) were compared with cells transfected only with the reporter constructs (panels 1 and 5).

FIG. 4 shows a quantitation of aggregation in different constructs from FIG. 3, normalized to the control cells expressing only the GFP-TDP43CTF construct.

FIG. 5 shows immunoblot analysis of extracts of cells expressing GFP reporter constructs with or without fusion protein constructs. The top panel is a Western blot analysis using anti-GFP antibodies, detecting the larger GFP-TDP43FL (lanes 1-4) and smaller GFP-TDP43CTF (lanes 5-8). The lower panel is a Western blot analysis using anti-FLAG epitope antibodies, which detects the scFv (3B12A) control (lanes 2 and 6), the fusion protein constructs (DnaJB1-scFv (3B12A), lanes 3 and 7), and the DnaJB1-scFv (3B12A) fusion protein containing a P33Q mutation in the conserved HPD domain (panels 4 and 8).

FIG. 6 shows immunoblot detection of the GFP reporter constructs from soluble or insoluble fractions from extracts of cells expressing either the GFP-TDP43FL or GFP-TDP43CTF reporter constructs, and also expressing none (negative control) or constructs 2 or 3.

FIG. 7 shows visualization of aggregation of TDP43-GFP fusion constructs in cells as measured by fluorescence microscopy, and the effect of J domain fusion proteins in reducing the aggregation. Fluorescence microscopy of cells transfected with GFP reporter constructs containing either full-length TDP-43 (GFP-TDP43FL) or the C-terminal fragment of TDP-43 (GFP-TDPCTF), and further containing constructs 2, 3, 5, 6, and 7 were compared with controls expressing reporter alone (none).

FIG. 8 shows visualization of aggregation of TDP43-GFP fusion constructs in cells as measured by fluorescence microscopy, and the effect of J domain fusion proteins in reducing the aggregation. Fluorescence microscopy of cells transfected with GFP reporter constructs containing either full-length TDP-43 (GFP-TDP43FL) or the C-terminal fragment of TDP-43 (GFP-TDPCTF), and further containing constructs 1, 2, 3, 4, 9, 10, 11 or 14 were compared with controls expressing reporter alone (none).

FIG. 9 shows quantitation and detection of TDP-43 reporter constructs: FIG. 9A shows quantitative differences in aggregation in cells from the experiment shown in FIG. 8.

FIG. 9B shows immunoblot analysis of cell extracts using anti-GFP antibody to quantitate the reporter construct levels in cellular extracts.

FIG. 10 shows the effects of Bafilomycin A1 (BFA), a potent inhibitor of late-phase autophagy and MG132, a proteasome inhibitor, on the reduction of the GFP-TDP43CTF in cells co-expressing Construct 3. Cells were transfected either with the reporter construct GFP-TDP43FL (lane 2) or GFP-TDP43CTF (lanes 3-8), and co-transfected with nucleic acids encoding Construct 3 (lanes 4-8). Treatment of cells with either BFA (at 0.01 μM, lane 5, and at 0.1 μM, lane 6) resulted in higher levels of the GFP-TDP43CTF reporter protein as detected by immunoblot. In contrast, treatment with 0.1 or 1.0 μM of MG132 (lanes 7 and 8, respectively) had little to no effect.

FIG. 11 shows the effects of co-expressing Construct 3 (lanes 4 and 8) on the levels of the GFP-TDP43CTF reporter in soluble (non-aggregated) and insoluble (aggregated) fractions of cell extracts, as probed with anti-TDP43 antibody (FIG. 11A) and anti-phospho TDP43 antibody (FIG. 11B)

FIG. 12 shows the effects of co-expression of Construct 3 (lane 3), Construct 7 (lane 4), Construct 2 (lane 5), Construct 15 (lane 6) and Construct 16 (lane 7) in reducing the level of phosphorylated GFP-TDP43CTF reporter in cell extracts, as probed with anti-phosphoTDP43 antibody.

FIG. 13 Shows the effects of co-expressing Construct 3 (lanes 3, 5, 8 and 10) in cells expressing either the TDP43FL (lanes 2, 3, 7 and 8) and TDP43ANLS constructs (lanes 4, 5, 9 and 10) on the levels of TDP-43 (as probed with anti-TDP43 antibodies, top panel), phosphorylated TDP-43 (as probed with anti-phospho TDP-43 antibodies, second panel), Flag epitope (probed with anti-FLAG antibody, third panel), and tubulin (anti-tubulin antibody, bottom panel).

FIG. 14 Shows additional constructs tested for the ability to reduce phosphorylated TDP-43. Cells expressing either GFP-TDP43FL (lanes 1 and 7) or GFP-TDP43CTF (lanes 2-6, 8-12) were co-transfected with Construct 3 (lanes 3 and 9), Construct 17 (lanes 4 and 10), Construct 18 (lanes 5 and 11) and Construct 19 (lanes 6 and 12). Soluble fractions (lanes 1-6) and insoluble fractions (lanes 7-12) were probed with anti-phospho TDP43 antibodies.

FIG. 15 shows a summary of the results of C57 mice injected with AAV rh10 containing either control or a vector encoding Construct 3, administered either by intrathecal (IT) or intracerebrovascular (ICV) injection. FIG. 15A summarizes the study schedule. FIG. 15B shows immunoblots of cerebrum extracts from mice 3 weeks after IT administration with AAV rh10 containing control (lanes 1 & 2) or a vector containing Construct 3 (lanes 3 & 4). FIG. 15C shows immunoblots of cerebrum extracts from mice after ICV injection. Lanes 1 —3 show immunoblots of cerebrum extracts from mice 3 weeks after ICV administration with AAV rh10 containing control (lanes 1 & 2) or a vector containing Construct 3 (lane 3). Lanes 4-8 show immunoblots of 8 week mice from control (lanes 4-6) and Construct 3 (lanes 7 & 8) mice.

FIG. 16 shows a summary of the results using the ANLS8 mice injected with AAV rh10 containing either control or vector encoding Construct 3, administered by ICV injection. FIG. 16A shows the study schedule. FIG. 16B shows the average weight of males from each group. FIG. 16C shows survival of mice from the different groups.

### DEFINITIONS

As used in the specification and claims, the singular forms “a”, “an” and “the” include plural references unless the context clearly dictates otherwise. For example, the term “a cell” includes a plurality of cells, including mixtures thereof.

The terms “polypeptide”, “peptide”, and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

As used herein the term “amino acid” refers to either natural and/or unnatural or synthetic amino acids, including but not limited to both the D or L optical isomers, and amino acid analogs and peptidomimetics. Standard single or three letter codes are used to designate amino acids.

A “host cell” includes an individual cell or cell culture which can be or has been a recipient for the subject vectors. Host cells include progeny of a single host cell. The progeny may not necessarily be completely identical (in morphology or in genomic of total DNA complement) to the original parent cell due to natural, accidental, or deliberate mutation. A host cell includes cells transfected in vivo with a vector of this invention.

“Isolated,” when used to describe the various polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is generally greater than that of its naturally occurring counterpart. In general, a polypeptide made by recombinant means and expressed in a host cell is considered to be “isolated.”

An “isolated” polynucleotide or polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal or extra-chromosomal location different from that of natural cells.

The terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The terms “TDP-43 disorder” or “TDP-43-mediated disease”, as herein defined refers to disorders associated with formation of intracellular TDP-43 aggregates, particularly aggregates of TDP-43 mutant protein. Examples of TDP-43 disorders include, but are not limited to preferably referring to amyotrophic lateral sclerosis (ALS), frontotemporal dementia (FTD), Parkinson's disease, Huntington's disease, Alzheimer's disease, hippocampal sclerosis, dementia with Lewy's bodies, and limbic predominant age-related TDP-43 encephalopathy.

A “vector” is a nucleic acid molecule, preferably self-replicating in an appropriate host, which transfers an inserted nucleic acid molecule into and/or between host cells. The term includes vectors that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions. An “expression vector” is a polynucleotide which, when introduced into an appropriate host cell, can be transcribed and translated into a polypeptide(s). An “expression system” usually connotes a suitable host cell comprised of an expression vector that can function to yield a desired expression product.

The term “operably linked” refers to a juxtaposition of described components wherein the components are in a relationship permitting them to function in their intended manner. A control sequence “operably linked” to a coding sequence is ligated in such a way that expression of the coding sequence is achieved under conditions compatible with the control sequences. “Operably linked” sequences may include both expression control sequences that are contiguous with the gene of interest and expression control sequences that act in trans or at a distance to control the gene of interest. The term “expression control sequence” refers to polynucleotide sequences that are necessary to affect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (such as, a Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include components whose presence is essential for expression and processing and can also include additional components whose presence is advantageous, for example, leader sequences and fusion partner sequences. Unless stated otherwise, a description or statement herein of inserting a nucleic acid molecule encoding a fusion protein of the invention into an expression vector means that the inserted nucleic acid has also been operably linked within the vector to a functional promoter and other transcriptional and translational control elements required for expression of the encoded fusion protein when the expression vector containing the inserted nucleic acid molecule is introduced into compatible host cells or compatible cells of an organism.

“Recombinant” as applied to a polynucleotide means that the polynucleotide is the product of various combinations of in vitro cloning, restriction and/or ligation steps, and other procedures that result in a construct that can potentially be expressed in a host cell.

The terms “gene” and “gene fragment” are used interchangeably herein. They refer to a polynucleotide containing at least one open reading frame that is capable of encoding a particular protein after being transcribed and translated. A gene or gene fragment may be genomic or cDNA, as long as the polynucleotide contains at least one open reading frame, which may cover the entire coding region or a segment thereof. A “fusion gene” is a gene composed of at least two heterologous polynucleotides that are linked together.

The terms “disease” and “disorder” are used interchangeably to indicate a pathological state identified according to acceptable medical standards and practices in the art.

As used herein, the term “effective amount” refers to the amount of a therapy that is sufficient to reduce or ameliorate the severity and/or duration of a disease or one or more symptoms thereof; to prevent the advancement of a detrimental or pathological state; to cause regression of a pathological state; to prevent recurrence, development, onset, or progression of one or more symptoms associated with a pathological state; to detect a disorder; or to enhance or improve the prophylactic or therapeutic effect(s) of a therapy (e.g., the administration of another prophylactic or therapeutic agent).

As used herein, the term “J domain” refers to a fragment which retains the ability to accelerate the intrinsic ATPase catalytic activity of Hsp70 and its cognate. The J domains of a variety of J proteins have been determined (see, for example, Kampinga et al. (2010) Nat. Rev., 11:579-592; Hennessy et al. (2005) Protein Science, 14:1697-1709, each of which is incorporated by reference in its entirety), and are characterized by a number of hallmarks: which is characterized by four α-helices (I, II, III, IV) and usually having the highly conserved tripeptide sequence motif of histidine, proline, and aspartic acid (referred to as the “HPD motif”) between helices II and III. Typically, the J domain of a J protein is between fifty and seventy amino acids in length, and the site of interaction (binding) of a J domain with an Hsp70-ATP chaperone protein is believed to be a region extending from within helix II and the HPD motif is necessary for stimulation of Hsp70 ATPase activity. As used herein, the term “J domain” is meant to include natural J domain sequences and functional variants thereof which retain the ability to accelerate Hsp70 intrinsic ATPase activity, which can be measured using methods well known in the art (see, for example, Horne et al. (2010) J. Biol. Chem., 285, 21679-21688, which is incorporated herein by reference in its entirety). A non-limiting list of human J domains is provided in Table 1.

### DETAILED DESCRIPTION

The present inventors have found that certain contacting cells with a fusion protein construct comprising a J domain of a J protein and a TDP-43-binding domain have the unexpected effect of reducing the aggregation of mutant TDP43 proteins. Aggregation of mutant TDP-43 are believed to cause a number of devastating diseases, including, but not limited to, amyotrophic lateral sclerosis (ALS), Frontotemporal Dementia, and Alzheimer's Disease. Accordingly, useful compositions and methods to treat TDP-43 disorders, e.g., in a subject in need thereof, are provided herein.

To overcome issues associated with chaperone-based therapies, we investigated whether it would be possible to design artificial chaperone proteins with high specificity. We designed a series of fusion protein constructs comprising an effector domain for Hsp70 binding/activation (J domain sequence), and a domain conferring specificity to TDP-43 proteins. The resulting fusion proteins act to accelerate the intrinsic ATPase catalytic activity of Hsp70 and its cognate, resulting in increased protein folding, reduced aggregation and/or accelerated clearance.

### I. Fusion Protein Constructs

a. J Domains Useful in the Invention

J domains of a variety of J proteins have been determined. See, for example, Kampinga et al., Nat. Rev., 11:579-592 (2010); Hennessy et al., Protein Science, 14:1697-1709 (2005). A J domain useful in preparing a fusion protein of the invention has the key defining features of a J domain which principally accelerates HSP70 ATPase activity. Accordingly, an isolated J domain useful in the invention comprises a polypeptide domain, which is characterized by four α-helices (I, II, III, IV) and usually having the highly conserved tripeptide sequence of histidine, proline, and aspartic acid (referred to as the “HPD motif”) between helices II and III. Typically, the J domain of a J protein is between fifty and seventy amino acids in length, and the site of interaction (binding) of a J domain with an Hsp70-ATP chaperone protein is believed to be a region extending from within helix II and the HPD motif is fundamental to primitive activity. Representative J domains include, but are not limited, a J domain of a DnaJB1, DnaJB2, DnaJB6, DnaJC6, a J domain of a large T antigen of SV40, and a J domain of a mammalian cysteine string protein (CSP-α). The amino acid sequences for these and other J domains that may be used in fusion proteins of the invention are provided in Table 1. The conserved HPD motif is highlighted in bold. In one embodiment, the fusion protein disclosed herein comprises a J domain selected from the group consisting of SEQ ID NOs: 1-50. As shown below in the Examples section, the inventors have discovered that use of a J domain missing the conserved “HPD” motif is not capable of reducing protein aggregation. As such, in another embodiment, the the fusion protein disclosed herein comprises a J domain comprising the consensus HPD motif. In one particular embodiment, selected from the group consisting of SEQ ID NOs: 1-15, 17-50.

In a particular embodiment, the fusion protein comprises a J domain selected from the group consisting of SEQ ID NOs: 1, 5, 6, 10, 16, 24, 25, 31 and 49.

TABLE 1
Representative Human J Domain Sequences

| Protein | SEQ ID | | |
|---|---|---|---|
| Name | NO: | Length | J domain amino acid sequence |
| DNAJA1 | 1 | 63 | TYYDVLGVKPNATQEELKKAYRKLALKYHPDKNPNEGEKFK |
| | | | QISQAYEVLSDAKKRELYDKGG |
| DNAJA2 | 2 | 63 | KLYDILGVPPGASENELKKAYRKLAKEYHPDKNPNAGDKEK |
| | | | EISFAYEVLSNPEKRELYDRYG |
| DNAJA3 | 3 | 66 | DYYQILGVPRNASQKEIKKAYYQLAKKYHPDTNKDDPKAKE |
| | | | KFSQLAEAYEVLSDEVKRKQYDAYG |
| DNAJA4 | 4 | 67 | ETQYYDILGVKPSASPEEIKKAYRKLALKYHPDKNPDEGEK |
| | | | FKLISQAYEVLSDPKKRDVYDQGGEQ |
| DNAJB1 | 5 | 69 | GKDYYQTLGLARGASDEEIKRAYRRQALRYHPDKNKEPGAE |
| | | | EKFKEIAEAYDVLSDPRKREIFDRYGEE |
| DNAJB2 | 6 | 70 | ASYYEILDVPRSASADDIKKAYRRKALQWHPDKNPDNKEFA |
| | | | EKKFKEVAEAYEVLSDKHKREIYDRYGRE |
| DNAJB3 | 7 | 69 | MVDYYEVLDVPRQASSEAIKKAYRKLALKWHPDKNPENKEE |
| | | | AERRFKQVAEAYEVLSDAKKRDIYDRYG |
| DNAJB4 | 8 | 69 | GKDYYCILGIEKGASDEDIKKAYRKQALKFHPDKNKSPQAE |
| | | | EKFKEVAEAYEVLSDPKKREIYDQFGEE |
| DNAJB5 | 9 | 65 | DYYKILGIPSGANEDEIKKAYRKMALKYHPDKNKEPNAEEK |
| | | | FKEIAEAYDVLSDPKKRGLYDQYG |
| DNAJB6 | 10 | 68 | VDYYEVLGVQRHASPEDIKKAYRKLALKWHPDKNPENKEEA |
| | | | ERKEKQVAEAYEVLSDAKKRDIYDKYG |
| DNAJB7 | 11 | 67 | DYYEVLGLQRYASPEDIKKAYHKVALKWHPDKNPENKEEAE |
| | | | RKFKEVAEAYEVLSNDEKRDIYDKYG |
| DNAJB8 | 12 | 67 | NYYEVLGVQASASPEDIKKAYRKLALRWHPDKNPDNKEEAE |
| | | | KKFKLVSEAYEVLSDSKKRSLYDRAG |
| DNAJB9 | 13 | 65 | SYYDILGVPKSASERQIKKAFHKLAMKYHPDKNKSPDAEAK |
| | | | FREIAEAYETLSDANRRKEYDTLG |
| DNAJB11 | 14 | 66 | DFYKILGVPRSASIKDIKKAYRKLALQLHPDRNPDDPQAQE |
| | | | KFQDLGAAYEVLSDSEKRKQYDTYG |
| DNAJB12 | 15 | 65 | YEILGVSRGASDEDLKKAYRRLALKFHPDKNHAPGATEAFK |
| | | | AIGTAYAVLSNPEKRKQYDQFGDD |
| DNAJB13 | 16 | 66 | DYYSVLGITRNSEDAQIKQAYRRLALKHHPLKSNEPSSAEI |
| | | | FRQIAEAYDVLSDPMKRGIYDKFG |
| DNAJB14 | 17 | 65 | NYYEVLGVTKDAGDEDLKKAYRKLALKFHPDKNHAPGATDA |
| | | | FKKIGNAYAVLSNPEKRKQYDLTG |
| DNAJC1 | 18 | 65 | NFYQFLGVQQDASSADIRKAYRKLSLTLHPDKNKDENAETQ |
| | | | FRQLVAIYEVLKDDERRQRYDDIL |
| DNAJC2 | 19 | 74 | DHYAVLGLGHVRYKATQRQIKAAHKAMVLKHHPDKRKAAGE |
| | | | PIKEGDNDYFTCITKAYEMLSDPVKRRAFNSVD |
| DNAJC3 | 20 | 69 | DYYKILGVKRNAKKQEIIKAYRKLALQWHPDNFQNEEEKKK |
| | | | AEKKFIDIAAAKEVLSDPEMRKKEDDGE |
| DNAJC4 | 21 | 66 | TYYELLGVHPGASTEEVKRAFFSKSKELHPDRDPGNPSLHS |
| | | | RFVELSEAYRVLSREQSRRSYDDQL |
| DNAJC5 | 22 | 70 | GESLYHVLGLDKNATSDDIKKSYRKLALKYHPDKNPDNPEA |
| | | | ADKFKEINNAHAILTDATKRNIYDKYGSL |
| DNAJC5B | 23 | 66 | ALYEILGLHKGASNEEIKKTYRKLALKHHPDKNPDDPAATE |
| | | | KFKEINNAHAILTDISKRSIYDKYG |
| DNAJC6 | 24 | 65 | TKWKPVGMADLVTPEQVKKVYRKAVLVVHPDKATGQPYEQY |
| | | | AKMIFMELNDAWSEFENQGQKPLY |
| DNAJC7 | 25 | 71 | DYYKILGVDKNASEDEIKKAYRKRALMHHPDRHSGASAEVQ |
| | | | KEEEKKFKEVGEAFTILSDPKKKTRYDSGQ |
| DNAJC8 | 26 | 68 | NPFEVLQIDPEVTDEEIKKRFRQLSILVHPDKNQDDADRAQ |
| | | | KAFEAVDKAYKLLLDQEQKKRALDVIQ |
| DNAJC9 | 27 | 68 | DLYRVLGVRREASDGEVRRGYHKVSLQVHPDRVGEGDKEDA |
| | | | TRRFQILGKVYSVLSDREQRAVYDEQG |
| DNAJC10 | 28 | 66 | DFYSLLGVSKTASSREIRQAFKKLALKLHPDKNPNNPNAHG |
| | | | DFLKINRAYEVLKDEDLRKKYDKYG |
| DNAJC11 | 29 | 69 | DYYSLLNVRREASSEELKAAYRRLCMLYHPDKHRDPELKSQ |
| | | | AERLENLVHQAYEVLSDPQTRAIYDIYG |
| DNAJC12 | 30 | 66 | DYYTLLGCDELSSVEQILAEFKVRALECHPDKHPENPKAVE |
| | | | TFQKLQKAKEILTNEESRARYDHWR |
| DNAJC13 | 31 | 66 | DAYEVLNLPQGQGPHDESKIRKAYFRLAQKYHPDKNPEGRD |
| | | | MFEKVNKAYEFLCTKSAKIVDGPDP |
| DNAJC14 | 32 | 65 | NPFHVLGVEATASDVELKKAYRQLAVMVHPDKNHHPRAEEA |
| | | | FKVLRAAWDIVSNAEKRKEYEMKR |
| DNAJC15 | 33 | 55 | EAGLILGVSPSAGKAKIRTAHRRVMILNHPDKGGSPYVAAK |
| | | | INEAKDLLETTTKH |
| DNAJC16 | 34 | 65 | DPYRVLGVSRTASQADIKKAYKKLAREWHPDKNKDPGAEDK |
| | | | FIQISKAYEILSNEEKRSNYDQYG |
| DNAJC17 | 35 | 66 | DLYALLGIEEKAADKEVKKAYRQKALSCHPDKNPDNPRAAE |
| | | | LFHQLSQALEVLTDAAARAAYDKVR |
| DNAJC18 | 36 | 65 | NYYEILGVSRDASDEELKKAYRKLALKFHPDKNCAPGATDA |
| | | | FKAIGNAFAVLSNPDKRLRYDEYG |
| DNAJC19 | 37 | 55 | EAALILGVSPTANKGKIRDAHRRIMLLNHPDKGGSPYIAAK |
| | | | INEAKDLLEGQAKK |
| DNAJC20 | 38 | 72 | DYFSLMDCNRSFRVDTAKLQHRYQQLQRLVHPDFFSQRSQT |
| | | | EKDFSEKHSTLVNDAYKTLLAPLSRGLYLLK |
| DNAJC21 | 39 | 67 | CHYEALGVRRDASEEELKKAYRKLALKWHPDKNLDNAAEAA |
| | | | EQFKLIQAAYDVLSDPQERAWYDNHR |
| DNAJC22 | 40 | 65 | LAYQVLGLSEGATNEEIHRSYQELVKVWHPDHNLDQTEEAQ |
| | | | RHFLEIQAAYEVLSQPRKPWGSRR |
| DNAJC23 | 41 | 62 | NPYEVLNLDPGATVAEIKKQYRLLSLKYHPDKGGDEVMEMR |
| | | | IAKAYAALTDEESRKNWEEFG |
| DNAJC24 | 42 | 72 | DWYSILGADPSANISDLKQKYQKLILMYHPDKQSTDVPAGT |
| | | | VEECVQKFIEIDQAWKILGNEETKREYDLQR |
| DNAJC25 | 43 | 76 | DCYEVLGVSRSAGKAEIARAYRQLARRYHPDRYRPQPGDEG |
| | | | PGRTPQSAEEAFLLVATAYETLKDEETRKDYDYML |
| DNAJC26 | 44 | 65 | SRWTPVGMADLVAPEQVKKHYRRAVLAVHPDKAAGQPYEQH |
| | | | AKMIFMELNDAWSEFENQGSRPLF |
| DNAJC27 | 45 | 57 | DSWDMLGVKPGASRDEVNKAYRKLAVLLHPDKCVAPGSEDA |
| | | | FKAVVNARTALLKNIK |
| DNAJC28 | 46 | 65 | EYYRLLNVEEGCSADEVRESFHKLAKQYHPDSGSNTADSAT |
| | | | FIRIEKAYRKVLSHVIEQTNASQS |
| DNAJC29 | 47 | 88 | ILKEVTSVVEQAWKLPESERKKIIRRLYLKWHPDKNPENHD |
| | | | IANEVFKHLQNEINRLEKQAFLDQNADRASRRTESTSASRF |
| | | | QSDKYS |
| DNAJC30 | 48 | 66 | ALYDLLGVPSTATQAQIKAAYYRQCFLYHPDRNSGSAEAAE |
| | | | RFTRISQAYVVLGSATLRRKYDRGL |
| SV40 | 49 | 64 | QLMDLLGLERSAWGNIPLMRKAYLKKCKEFHPDKGGDEEKM |
| Jdomain | | | KKMNTLYKKMEDGVKYAHQPDFG |
| Bacterial | 50 | 70 | KQDYYEILGVSKTAEEREIRKAYKRLAMKYHPDRNQGDKEA |
| J-domain | | | EAKFKEIKEAYEVLTDSQKRAAYDQYGHA |

b. TDP-43-binding domain

The fusion protein also comprises at least one TDP-43-binding domain. The TDP-43-binding domain can be a single chain polypeptide, or a multimeric polypeptide joined with the J domain to form the fusion protein.

It is ideal that the TDP-43-binding domain possesses a sufficient affinity to be able to bind the TDP-43 protein when present at a pathological level within cells. Therefore, in one embodiment, the fusion protein comprises a TDP-43-binding domain that has a Kp for TDP-43 reporter construct (for example, the full-length TDP-43 (Novus Biologicals, NBP2-22850, Centennial, CO) of, for example, 2 μM or less, 1 μM or less, 500 nM or less, 300 nM or less, 100 nM or less, 30 nM or less when tested by ELISA on 96 well microtiter plates.

TDP-43-binding domains have been previously identified and characterized (see, for example, U.S. Pat. No. 10,259,866, WO 2016/53610, WO 2018/218252, WO 2019/134981, WO 2019/177138, each of which is incorporated herein by reference). Therefore, in another embodiment, the fusion protein comprises a TDP-43-binding domain that is selected from the group consisting of SEQ ID NOs: 51-55 (see, for example, Table 2). In one particular embodiment, the fusion protein comprises the TDP-43-binding domain of SEQ ID NO: 51. In another embodiment, the fusion protein comprises the TDP-43-binding domain of SEQ ID NO: 53. In still another embodiment, the fusion protein comprises the TDP-43-binding domain of SEQ ID NO: 52.

In another embodiment, the fusion protein also contemplates the use of the TDP-43-binding domain that is chemically conjugated to the J domain. The TDP-43-binding domain can be conjugated directly to the J domain. Alternatively, it can be conjugated to the J domain by a linker. For example, there are a large number of chemical cross-linking agents that are known to those skilled in the art and useful for cross-linking the TDP-43-binding domain to the J domain, or a targeting domain to a fusion protein comprising the TDP-43-binding domain and J domain. For example, the cross-linking agents are heterobifunctional cross-linkers, which can be used to link molecules in a stepwise manner. Heterobifunctional cross-linkers provide the ability to design more specific coupling methods for conjugating proteins, thereby reducing the occurrences of unwanted side reactions such as homo-protein polymers. A wide variety of heterobifunctional cross-linkers are known in the art, including succinimidyl 4-(N-maleimidomethyl) cyclohexane-1-carboxylate (SMCC), m-Maleimidobenzoyl-N-hydroxysuccinimide ester (MBS); N-succinimidyl (4-iodoacetyl) aminobenzoate (SIAB), succinimidyl 4-(p-maleimidophenyl) butyrate (SMPB), 1-ethyl-3-(3-dimethylaminopropyl) carbodiimide hydrochloride (EDC); 4-succinimidyloxycarbonyl-a-methyl-a-(2-pyridyldithio)-toluene (SMPT), N-succinimidyl 3-(2-pyridyldithio) propionate (SPDP), succinimidyl 6-[3-(2-pyridyldithio) propionate] hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo. In addition to the heterobifunctional cross-linkers, there exists a number of other cross-linking agents including homobifunctional and photoreactive cross-linkers. Disuccinimidyl suberate (DSS), bismaleimidohexane (BMH) and dimethylpimelimidate.2 HCl (Forbes-Cori Disease) are examples of useful homobifunctional cross-linking agents, and bis-[B-(4-azidosalicylamido) ethyl] disulfide (BASED) and N-succinimidyl-6 (4′-azido-2′-nitrophenylamino) hexanoate (SANPAH) are examples of useful photoreactive cross-linkers for use in this disclosure. For a recent review of protein coupling techniques, see Means et al., (1990) Bioconj. Chem. 1:2-12, incorporated by reference herein.

TABLE 2
Examples of TDP-43-binding domains

| | SEQ ID | | |
|---|---|---|---|
| Name | NO: | Length | Sequence |
| 3B12A | 51 | 241 | EVQLQQSGAELVKPGASVKL |
| | | | SCTASGFNIKDYYMHWVKQR |
| | | | TEQGLEWIGRIDPEDGETKY |
| | | | APKFQGKATITADTSSNTAY |
| | | | LQLSSLTSEDTAVYYCTIIY |
| | | | YYGSRYVDYWGQGTTLTVSG |
| | | | GGGSGGGGSGGGGSEIVLTQ |
| | | | SPTTMAASPGEKITITCSAS |
| | | | SSISSSYLHWYQQKPGESPK |
| | | | LLIYRTSNLASGVPARFSGS |
| | | | GSGTSYSLTIGTMEAEDVAT |
| | | | YYCQQGSSIPLTFGSGTKLE |
| | | | I |
| 51C1 | 52 | 239 | EVQLVESGGGLVQPGGSLRI |
| | | | SCTTSGFIFSDYWMHWVRQA |
| | | | PGKGLTWVSRINLDGSDTIY |
| | | | ADSVKGRFTISRDNDKNTLY |
| | | | LQMNSLRVEDTAIYYCARSR |
| | | | KSVWGQGTMVTVSSGGGGSG |
| | | | GGGSGGGGSQSALTQPASVS |
| | | | GSPGQSITISCTGSNTDVGA |
| | | | YDYVSWSQQLPGKAPKFVIF |
| | | | DVDVRPSGISDRESGSKSGN |
| | | | TASLTISGLQAEDEADYYCS |
| | | | SYTKSGTLVEGGGTKVTVV |
| 3F10 | 53 | 248 | EVQLLESGGDLVQPGGSLRL |
| | | | SCAASGFTESSQAMSWVRQA |
| | | | PGKGLEWVSALSRTGDYTWY |
| | | | ADSVRGRFTVSRDDSKNIFY |
| | | | LEMNSLRAEDTAVYYCAKNY |
| | | | YSSFGYNWAAFHIWGQGTMV |
| | | | TVSSGGGGSGGGGSGGGGSE |
| | | | IVLTQSPGTLSLSPGERATL |
| | | | SCRASQDVNNNYLAWYQQKP |
| | | | GQAPRLLIYGASRRATGVPD |
| | | | RFSGRGSGTDFTLTINRLEP |
| | | | EDFAMYFCQQYGGSPPYTFG |
| | | | QGTKLEIK |
| PolyQ | 54 | 25 | QC |
| (Q25) | | | |
| QBP1 | 55 | 11 | SNWKWWPGIFD |

c. Optional Linker

The fusion proteins described herein can optionally contain one or more linkers. Linkers can be peptidic or non-peptidic. The purpose of the linker is to provide, among other things, an adequate distance between functional domains within the protein (e.g., between the J domain and TDP-43-binding domain, between tandem arrangements of TDP-43-binding domains, between either the J domain and TDP-43-binding domain and an optional targeting reagent, or between either the J domain and TDP-43-binding domain and an optional detection domain or epitope) for optimal function of each of the domains. Clearly, a linker preferably does not interfere with the respective functions of the J domain, the target protein binding domain of a fusion protein according to the invention. A linker, if present in a fusion protein of the invention, is selected to attenuate the cytotoxicity caused by target proteins (TDP-43 proteins), and it may be omitted if direct attachment achieves a desired effect. Linkers present in a fusion protein of the invention may comprise one or more amino acids encoded by a nucleotide sequence present on a segment of nucleic acid in or around a cloning site of an expression vector into which is inserted in frame a nucleic acid segment encoding a protein domain or an entire fusion protein as described herein. In one embodiment, the peptide linker is between 1 amino acid and 20 amino acids in length. In another embodiment, the peptide linker is between 2 amino acids and 15 amino acids in length. In still another embodiment, the peptide linker is between 2 amino acids and 10 amino acids in length.

Selecting one or more polypeptide linkers to produce a fusion protein according to the invention is within the knowledge and skill of practitioners in the art. See, for example, Arai et al., Protein Eng., 14 (8): 529-532 (2001); Crasto et al., Protein Eng., 13 (5): 309-314 (2000); George et al., Protein Eng., 15 (11): 871-879 (2003); Robinson et al., Proc. Natl. Acad. Sci. USA, 95:5929-5934 (1998), each of which is incorporated herein by reference in its entirety. Examples of linkers of two or more amino acids that may be used in preparing a fusion protein according to the invention, include, by are not limited to, those provided below in Table 3.

TABLE 3
Linker Sequences

| SEQ | | |
|---|---|---|
| ID | | |
| NO: | Length | Sequence |
| 56 | 2 | SR |
| 57 | 4 | GTGS |
| 58 | 5 | GLESR |
| 59 | 4 | GGSG |
| 60 | 4 | GGGS |
| 61 | 5 | DIAAA |
| 62 | 9 | DIAAALESR |
| 63 | 15 | GGGGSGGGGSGGGGS |
| 64 | 11 | AEAAAKEAAAK |
| 65 | 15 | SGGGSGGGGSGGGGS |
| 66 | 25 | DIGGGGSGGGGSGGGGSGGGGSAAA |

d. Targeting Reagents

The fusion proteins disclosed herein can further comprise a targeting moiety. As used herein, the terms “targeting moiety” and “targeting reagent” are used interchangeably and refer to a substance associated with the fusion protein that enhances binding, transport, accumulation, residence time, bioavailability, or modifies biological activity or therapeutic effect of the fusion protein in a cell or in the body of a subject. A targeting moiety can have functionality at the tissue, cellular, and/or subcellular level. The targeting moiety can direct localization of the fusion protein to a particular cell, tissue or organ, or intracellular distribution, for example, upon administration of the fusion protein into a subject. In one embodiment, the targeting moiety is located at the N-terminus of the fusion protein. In another embodiment, the targeting moiety is located at the C-terminus of the fusion protein. In still another embodiment, the targeting moiety is located internally. In another embodiment, the targeting moiety is attached to the fusion protein via chemical conjugation.

The targeting moiety can include, but is not limited to, an organic or inorganic molecule, a peptide, a peptide mimetic, a protein, an antibody or fragment thereof, a growth factor, an enzyme, a lectin, an antigen or immunogen, viruses or component thereof, a viral vector, a receptors, a receptor ligand, a toxins, a polynucleotide, an oligonucleotide or aptamer, a nucleotide, a carbohydrate, a sugar, a lipid, a glycolipid, a nucleoprotein, a glycoprotein, a lipoprotein, a steroid, a hormone, a growth factor, a chemoattractant, a cytokine, a chemokine, a drug, or a small molecule, among others.

In an exemplary embodiment of the present invention, the targeting moiety enhances binding, transport, accumulation, residence time, bioavailability, or modifies biological activity of the modifies biological activity or therapeutic effect of the platform, or its associated ligand and/or active agent in the target cell or tissue, for example, neuronal cells, the central nervous system, and/or the peripheral nervous system. Thus, the targeting moiety can have specificity for cellular receptors associated with the central nervous system, or is otherwise associated with enhanced delivery to the CNS via the blood-brain barrier (BBB). Consequently, a ligand, as described above, can be both a ligand and a targeting moiety.

In some embodiments, the targeting moiety can be a cell-penetrating peptide, for example, as described in U.S. Pat. No. 10,111,965, which is incorporated by reference in its entirety. In another embodiment, the targeting moiety can be an antibody or an antigen-binding fragment or single-chain derivative thereof, for example, as described in U.S. Ser. No. 16/131,591, which is incorporated herein by reference in its entirety. In further embodiment, the targeting moiety can be a amino acid sequence for nuclear localization signal or nuclear export signal.

The targeting moiety can be coupled to the platform for targeted cellular delivery by being directly or indirectly bound to the core. For example, in embodiments where the core comprises a nanoparticle, conjugation of the targeting moiety to the nanoparticle can utilize similar functional groups that are employed to tether PEG to the nanoparticle. Thus, the targeting moiety can be directly bound to the nanoparticle through functionalization of the targeting moiety. Alternatively, the targeting moiety can be indirectly bound to the nanoparticle through conjugation of the targeting moiety to a functionalized PEG, as discussed above. A targeting moiety can be attached to core by way of covalent, non-covalent, or electrostatic interactions. In one embodiment, the targeting moiety is a peptide. In a particular embodiment, the targeting moiety is a peptide that is covalently attached to the N-terminus of the fusion protein.

e. Epitopes

In certain embodiments, the fusion protein of the present invention contains an optional epitope or tag, which can impart additional properties to the fusion protein. As used herein, the terms “epitope” and “tag” are used interchangeably to refer to an amino acid sequence, typically 300 amino acids or less in length, which is typically attached to the N-terminal or C-terminal end of the fusion protein. In one embodiment, the fusion protein of the present invention further comprises an epitope which is used to facilitate purification. Examples of such epitopes useful for purification, provided below in Table 4, include the human IgG1 Fc sequence (SEQ ID NO: 67), the FLAG epitope (DYKDDDDK, SEQ ID NO: 68), His6 epitope (SEQ ID NO: 69), c-myc (SEQ ID NO: 70), HA (SEQ ID NO: 71), V5 epitope (SEQ ID NO: 72), or glutathione-s-transferase (SEQ ID NO: 73). In another embodiment, the fusion protein of the present invention further comprises an epitope which is used to increase the half-life of the fusion protein when administered into a subject, for example a human. Examples of such epitopes useful for increasing half-life include the human Fc sequence. Therefore, in one particular embodiment, the fusion protein comprises, in addition to a J domain and TDP-43-binding domain, a human Fc epitope. The epitope is positioned at the C-terminal end of the fusion protein.

TABLE 4
Representative Examples of Epitopes

| SEQ | | | |
|---|---|---|---|
| ID | | | |
| NO: | EPITOPE | LENGTH | SEQUENCE |
| 67 | Human IgG1 | 232 | EPKSCDKTHTCPPCPAPELL |
| | Fc domain | | GGPSVFLFPPKPKDTLMISR |
| | | | TPEVTCVVVDVSHEDPEVKF |
| | | | NWYVDGVEVHNAKTKPREEQ |
| | | | YNSTYRVVSVLTVLHQDWLN |
| | | | GKEYKCKVSNKALPAPIEKT |
| | | | ISKAKGQPREPQVYTLPPSR |
| | | | DELTKNQVSLTCLVKGFYPS |
| | | | DIAVEWESNGQPENNYKTTP |
| | | | PVLDSDGSFFLYSKLTVDKS |
| | | | RWQQGNVFSCSVMHEALHNH |
| | | | YTQKSLSLSPGK |
| 68 | FLAG epitope | 8 | DYKDDDDK |
| 69 | His6 | 6 | HHHHHH |
| 70 | c-myc | 10 | EQKLISEEDL |
| 71 | HA | 9 | YPYDVPDYA |
| 72 | V5 epitope | 14 | GKPIPNPLLGLDST |
| 73 | Glutathione- | 220 | MSPILGYWKIKGLVQPTRLL |
| | S- | | LEYLEEKYEEHLYERDEGDK |
| | transferase | | WRNKKFELGLEFPNLPYYID |
| | | | GDVKLTQSMAIIRYIADKHN |
| | | | MLGGCPKERAEISMLEGAVL |
| | | | DIRYGVSRIAYSKDFETLKV |
| | | | DFLSKLPEMLKMFEDRLCHK |
| | | | TYLNGDHVTHPDFMLYDALD |
| | | | VVLYMDPMCLDAFPKLVCFK |
| | | | KRIEAIPQIDKYLKSSKYIA |
| | | | WPLQGWQATFGGGDHPPKSD |

f. Cell-Penetrating Peptides

In still other embodiments, the fusion protein described herein can further comprise a cell-penetrating peptide. Cell-penetrating peptides are known to carry a conjugated cargo, whether a small molecule, peptide, protein or nucleic acid, into cells. Non-limiting examples of cell-penetrating peptides in a fusion protein of the invention include, but are not limited to, a polycationic peptide, e.g., an HIV TAT peptide49-57, polyarginines, and penetratin pAntan (43-58), amphipathic peptide, e.g., pep-1, a hydrophobic peptide, e.g., a C405Y, and the like. See Table 5 below.

TABLE 5
Examples of Cell-Penetrating Peptides

| SEQ ID NO: | LENGTH | SEQUENCE |
|---|---|---|
| 74 | 9 | RKKRRQRRR |
| 75 | 15 | RQIKWFQNRRMKWKK |
| 76 | 21 | KETWWETWWTEWSQPKKKRKV |
| 77 | 17 | CSIPPEVKFNKPFVYLI |

Therefore, in one embodiment, the fusion protein comprises a cell-penetrating peptide and a fusion protein, wherein the cell-penetrating peptide is selected from the group consisting of SEQ ID NOs: 74-77, and the fusion protein comprising a J domain and a TDP-43 binding domain. In one embodiment, the fusion protein is selected from the group consisting of SEQ ID NOs: 80-85 and 89-96. In another embodiment, the fusion protein comprises the signal sequence of SEQ ID NO: 74, and the fusion protein selected from the group consisting of SEQ ID NOs: 80-85, 89-90 and 92-96. In another embodiment, the fusion protein comprises the cell-penetrating peptide of SEQ ID NO: 75, and the fusion protein selected from the group consisting of SEQ ID NOs: 80-85, 89-90 and 92-96. In still another embodiment, the fusion protein comprises the cell-penetrating peptide of SEQ ID NO: 76, and the fusion protein selected from the group consisting of SEQ ID NOs: 80-85, 89-90 and 92-96. In yet another embodiment, the fusion protein comprises the cell-penetrating peptide of SEQ ID NO: 77, and the fusion protein selected from the group consisting of SEQ ID NOs: 80-85, 89-90 and 92-96. Cells expressing the fusion protein constructs with the cell-penetrating peptide can be administered to a subject, for example a human subject (e.g., a patient having or at risk of suffering from a TDP-43 disorder). The fusion protein is secreted from the cells, which help reduce TDP-43-containing protein aggregation and/or associated cytotoxicity.

g. Arrangement of J Domain and TDP-43 Binding Domain

The fusion proteins described herein can be arranged in a multitude of ways. In one embodiment, the TDP-43-binding domains attached to the C-terminal side of the J domain. In another embodiment, the TDP-43-binding domains attached to the N-terminal side of the J domain. The TDP-43-binding domain and the J domain, in either configuration, can optionally be separated via a linker as described above.

In some embodiments, the J domain can be attached to a plurality of TDP-43-binding domains, for example, two TDP-43-binding domains, three TDP-43-binding domains, four TDP-43-binding domains or more. The TDP-43-binding domains can be attached to the N-terminal side of the J domain. Alternatively, the TDP-43-binding domains can be attached to the C-terminal side of the J domain. In still another embodiment, the TDP-43-binding domains can be attached on the N-terminal and C-terminal sides of the J domain. Each of the plurality of TDP-43-binding domains can be the same TDP-43-binding domain. In another embodiment, each of the plurality of TDP-43-binding domains in the fusion protein can be different TDP-43-binding domains (i.e., different sequences).

In some embodiments, the fusion proteins can comprise a structure selected from the following group:
a. DNAJ-X-T,
b. DNAJ-X-T-X-T,
c. DNAJ-X-T-X-T-X-T,
d. T-X-DNAJ,
e. T-X-T-X-DNAJ,
f. T-X-T-X-T-X-DNAJ,
g. T-X-DNAJ-X-T,
h. T-X-DNAJ-X-T-X-T,
i. TDNAJ-X-TTTTTDNAJ-X-T,
j. T-X-T-X-DNAJ-X-TT,
k. TTDNAJ-X-T-X-TTTTTDNAJ-X-T,
l. T-X-T-X-DNAJ-X-T-X-T-X-T,
m. T-X-T-X-T-X-DNAJ-X-T,
n. T-X-T-X-T-X-DNAJ-X-T-X-T,
o. T-X-T-X-T-X-DNAJ-X-T-X-T-X-T,
p. DnaJ-X-DnaJ-X-T-X-T,
q. T-X-DnaJ-X-DnaJ,
r. T-X-T-X-DnaJ-X-DnaJ, and
s. T-X-TDnaJ-X-TDnaJ-X-TTTT
wherein,
T is a TDP-43-binding domain,
DNAJ is a J domain of a J protein, and
X is an optional linker.

In one embodiment, the fusion protein comprises the J domain selected from the group consisting of SEQ ID NOs: 5, 6, 10, 24, and 31. In one particular embodiment, the fusion protein comprises the J domain of SEQ ID NO: 5.

In another embodiment, the TDP-43-binding domain is selected from the group consisting of SEQ ID NOs: 51-55. In one particular embodiment, the TDP-43-binding domain is selected from the group consisting of SEQ ID NOs: 51-53.

In still another embodiment, the fusion protein comprises the J domain of SEQ ID NO: 5, and the TDP-43-binding domain of SEQ ID NO: 51. In another embodiment, the fusion protein comprises the J domain of SEQ ID NO: 5, and at least two copies of the TDP-43-binding domain of SEQ ID NO: 53.

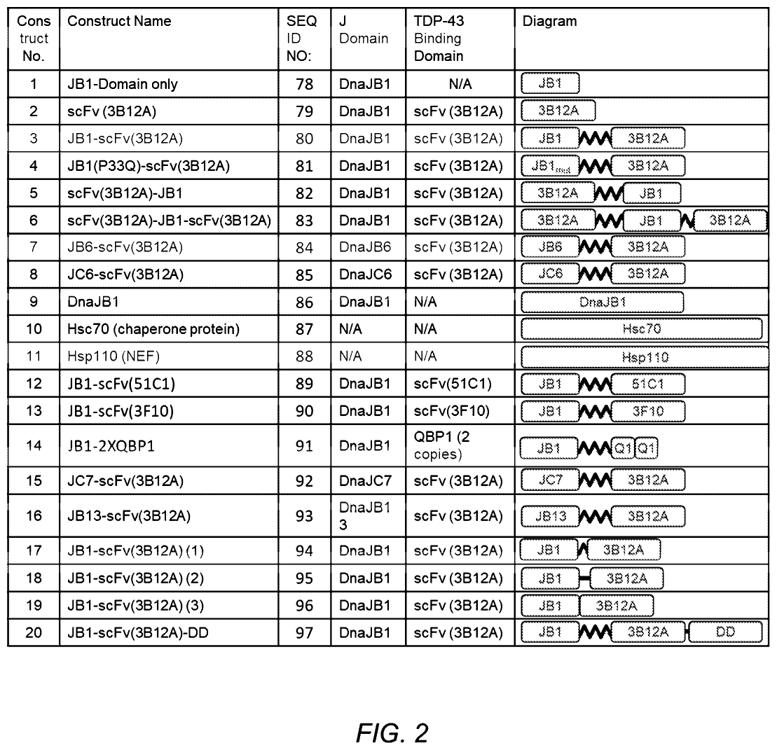

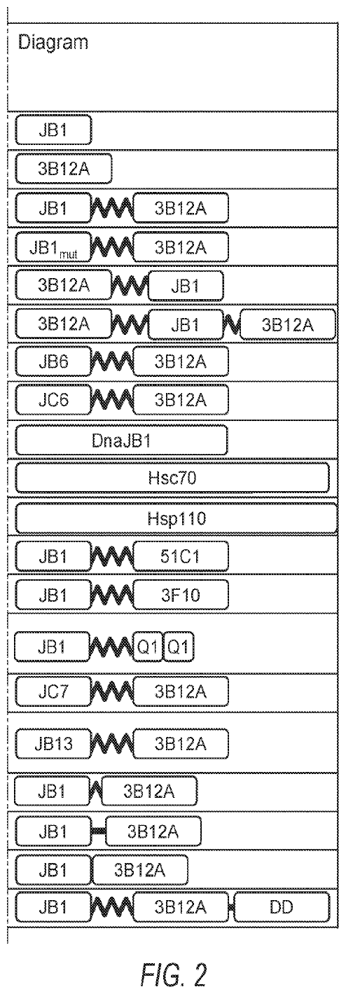

Non-limiting examples of fusion protein constructs comprising a J domain and TDP-43-binding domain are depicted schematically in FIG. 2, and also shown below in Table 6. In another embodiment, the specific fusion protein construct is selected from the group consisting of SEQ ID NOs: 80-85 and 89-96.

TABLE 6
Fusion Protein Constructs and Control Constructs

| Construct | SEQ ID | Construct | | |
|---|---|---|---|---|
| No | NO: | Name | Length | Sequence |
| 1 | 78 | J-domain | 75 | MGKDYYQTLGLARGASDEEIKRAYRRQALRYHPDKNKEPGAE |
| | | only | | EKFKEIAEAYDVLSDPRKREIFDRYGEEGLKGS |
| 2 | 79 | scFv(3B12A) | 253 | MGTGSEFDIAAAEVQLQQSGAELVKPGASVKLSCTASGENIK |
| | | | | DYYMHWVKQRTEQGLEWIGRIDPEDGETKYAPKFQGKATITA |
| | | | | DTSSNTAYLQLSSLTSEDTAVYYCTIIYYYGSRYVDYWGQGT |
| | | | | TLTVSGGGGSGGGGGGGGSEIVLTQSPTTMAASPGEKITIT |
| | | | | CSASSSISSSYLHWYQQKPGESPKLLIYRTSNLASGVPARES |
| | | | | GSGSGTSYSLTIGTMEAEDVATYYCQQGSSIPLTFGSGTKLE |
| | | | | I |
| 3 | 80 | JB1- | 341 | MGKDYYQTLGLARGASDEEIKRAYRRQALRYHPDKNKEPGAE |
| | | scFv(3B12A) | | EKFKEIAEAYDVLSDPRKREIFDRYGEEGLKGSDIGGGGSGG |
| | | | | GGSGGGGSGGGGSAAAEVQLQQSGAELVKPGASVKLSCTASG |
| | | | | FNIKDYYMHWVKQRTEQGLEWIGRIDPEDGETKYAPKFQGKA |
| | | | | TITADTSSNTAYLQLSSLTSEDTAVYYCTIIYYYGSRYVDYW |
| | | | | GQGTTLTVSGGGGSGGGGSGGGGSEIVLTQSPTTMAASPGEK |
| | | | | ITITCSASSSISSSYLHWYQQKPGFSPKLLIYRTSNLASGVP |
| | | | | ARFSGSGSGTSYSLTIGTMEAEDVATYYCQQGSSIPLTFGSG |
| | | | | TKLEI |
| 4 | 81 | JB1(P33Q)- | 341 | MGKDYYQTLGLARGASDEEIKRAYRRQALRYHQDKNKEPGAE |
| | | scFv(3B12A) | | EKFKEIAEAYDVLSDPRKREIFDRYGEEGLKGSDIGGGGSGG |
| | | | | GGSGGGGSGGGGSAAAEVQLQQSGAELVKPGASVKLSCTASG |
| | | | | FNIKDYYMHWVKQRTEQGLEWIGRIDPEDGETKYAPKFQGKA |
| | | | | TITADTSSNTAYLQLSSLTSEDTAVYYCTIIYYYGSRYVDYW |
| | | | | GQGTTLTVSGGGGSGGGGSGGGGSEIVLTQSPTTMAASPGEK |
| | | | | ITITCSASSSISSSYLHWYQQKPGFSPKLLIYRTSNLASGVP |
| | | | | ARFSGSGSGTSYSLTIGTMEAEDVATYYCQQGSSIPLTFGSG |
| | | | | TKLEI |
| 5 | 82 | scFv(3B12A)- | 323 | MGTEVQLQQSGAELVKPGASVKLSCTASGFNIKDYYMHWVKQ |
| | | JB1 | | RTEQGLEWIGRIDPEDGETKYAPKFQGKATITADTSSNTAYL |
| | | | | QLSSLTSEDTAVYYCTIIYYYGSRYVDYWGQGTTLTVSGGGG |
| | | | | SGGGGSGGGGSEIVLTQSPTTMAASPGEKITITCSASSSISS |
| | | | | SYLHWYQQKPGESPKLLIYRTSNLASGVPARFSGSGSGTSYS |
| | | | | LTIGTMEAEDVATYYCQQGSSIPLTFGSGTKLEIGSEFMGKD |
| | | | | YYQTLGLARGASDEEIKRAYRRQALRYHPDKNKEPGAEEKFK |
| | | | | EIAEAYDVLSDPRKREIFDRYGEEGLKGS |
| 6 | 83 | scFv(3B12A)- | 589 | MGTEVQLQQSGAELVKPGASVKLSCTASGFNIKDYYMHWVKQ |
| | | JB1- | | RTEQGLEWIGRIDPEDGETKYAPKFQGKATITADTSSNTAYL |
| | | scFv(3B12A) | | QLSSLTSEDTAVYYCTIIYYYGSRYVDYWGQGTTLTVSGGGG |
| | | | | SGGGGSGGGGSEIVLTQSPTTMAASPGEKITITCSASSSISS |
| | | | | SYLHWYQQKPGFSPKLLIYRTSNLASGVPARFSGSGSGTSYS |
| | | | | LTIGTMEAEDVATYYCQQGSSIPLTFGSGTKLEIGSEFMGKD |
| | | | | YYQTLGLARGASDEEIKRAYRRQALRYHPDKNKEPGAEEKFK |
| | | | | EIAEAYDVLSDPRKREIFDRYGEEGLKGSDIGGGGSGGGGSG |
| | | | | GGGSGGGGSAAAEVQLQQSGAELVKPGASVKLSCTASGENIK |
| | | | | DYYMHWVKQRTEQGLEWIGRIDPEDGETKYAPKFQGKATITA |
| | | | | DTSSNTAYLQLSSLTSEDTAVYYCTIIYYYGSRYVDYWGQGT |
| | | | | TLTVSGGGGSGGGGSGGGGSEIVLTQSPTTMAASPGEKITIT |
| | | | | CSASSSISSSYLHWYQQKPGESPKLLIYRTSNLASGVPARES |
| | | | | GSGSGTSYSLTIGTMEAEDVATYYCQQGSSIPLTFGSGTKLE |
| | | | | I |
| 7 | 84 | JB6- | 342 | MVDYYEVLGVQRHASPEDIKKAYRKLALKWHPDKNPENKEEA |
| | | scFv(3B12A) | | ERKEKQVAEAYEVLSDAKKRDIYDKYGKEGLNGGDIGGGGSG |
| | | | | GGGSGGGGSGGGGSAAAEVQLQQSGAELVKPGASVKLSCTAS |
| | | | | GFNIKDYYMHWVKQRTEQGLEWIGRIDPEDGETKYAPKFQGK |
| | | | | ATITADTSSNTAYLQLSSLTSEDTAVYYCTIIYYYGSRYVDY |
| | | | | WGQGTTLTVSGGGGSGGGGSGGGGSEIVLTQSPTTMAASPGE |
| | | | | KITITCSASSSISSSYLHWYQQKPGFSPKLLIYRTSNLASGV |
| | | | | PARFSGSGSGTSYSLTIGTMEAEDVATYYCQQGSSIPLTFGS |
| | | | | GTKLEI |
| 8 | 85 | JC6- | 346 | MTKWKPVGMADLVTPEQVKKVYRKAVLVVHPDKATGQPYEQY |
| | | scFv(3B12A) | | AKMIFMELNDAWSEFENQGQKPLYDIYDKYGKEGLNGGDIGG |
| | | | | GGSGGGGSGGGGSGGGGSAAAEVQLQQSGAELVKPGASVKLS |
| | | | | CTASGFNIKDYYMHWVKQRTEQGLEWIGRIDPEDGETKYAPK |
| | | | | FQGKATITADTSSNTAYLQLSSLTSEDTAVYYCTIIYYYGSR |
| | | | | YVDYWGQGTTLTVSGGGGSGGGGSGGGGSEIVLTQSPTTMAA |
| | | | | SPGEKITITCSASSSISSSYLHWYQQKPGESPKLLIYRTSNL |
| | | | | ASGVPARFSGSGSGTSYSLTIGTMEAEDVATYYCQQGSSIPL |
| | | | | TFGSGTKLEI |
| 9 | 86 | DnaJB1 | 343 | MGTMGKDYYQTLGLARGASDEEIKRAYRRQALRYHPDKNKEP |
| | | | | GAEEKFKEIAEAYDVLSDPRKREIFDRYGEEGLKGSGPSGGS |
| | | | | GGGANGTSFSYTFHGDPHAMFAEFFGGRNPFDTFFGQRNGEE |
| | | | | GMDIDDPFSGFPMGMGGFTNVNFGRSRSAQEPARKKQDPPVT |
| | | | | HDLRVSLEEIYSGCTKKMKISHKRLNPDGKSIRNEDKILTIE |
| | | | | VKKGWKEGTKITFPKEGDQTSNNIPADIVFVLKDKPHNIFKR |
| | | | | DGSDVIYPARISLREALCGCTVNVPTLDGRTIPVVFKDVIRP |
| | | | | GMRRKVPGEGLPLPKTPEKRGDLIIEFEVIFPERIPQTSRTV |
| | | | | LEQVLPI |
| 10 | 87 | Hsp70 | 840 | MSVVGIDLGFQSCYVAVARAGGIETIANEYSDRCTPACISFG |
| | | | | PKNRSIGAAAKSQVISNAKNTVQGFKRFHGRAFSDPFVEAEK |
| | | | | SNLAYDIVQLPTGLTGIKVTYMEEERNFTTEQVTAMLLSKLK |
| | | | | ETAESVLKKPVVDCVVSVPCFYTDAERRSVMDATQIAGLNCL |
| | | | | RLMNETTAVALAYGIYKQDLPALEEKPRNVVFVDMGHSAYQV |
| | | | | SVCAFNRGKLKVLATAFDTTLGGRKFDEVLVNHFCEEFGKKY |
| | | | | KLDIKSKIRALLRLSQECEKLKKLMSANASDLPLSIECEMND |
| | | | | VDVSGTMNRGKFLEMCNDLLARVEPPLRSVLEQTKLKKEDIY |
| | | | | AVEIVGGATRIPAVKEKISKFFGKELSTTLNADEAVTRGCAL |
| | | | | QCAILSPAFKVREFSITDVVPYPISLRWNSPAEEGSSDCEVE |
| | | | | SKNHAAPFSKVLTFYRKEPFTLEAYYSSPQDLPYPDPAIAQF |
| | | | | SVQKVTPQSDGSSSKVKVKVRVNVHGIFSVSSASLVEVHKSE |
| | | | | ENEEPMETDQNAKEEEKMQVDQEEPHVEEQQQQTPAENKAES |
| | | | | EEMETSQAGSKDKKMDQPPQAKKAKVKTSTVDLPIENQLLWQ |
| | | | | IDREMLNLYIENEGKMIMQDKLEKERNDAKNAVEEYVYEMRD |
| | | | | KLSGEYEKFVSEDDRNSFTLKLEDTENWLYEDGEDQPKQVYV |
| | | | | DKLAELKNLGQPIKIRFQESEERPKLFEELGKQIQQYMKIIS |
| | | | | SFKNKEDQYDHLDAADMTKVEKSTNEAMEWMNNKLNLQNKQS |
| | | | | LTMDPVVKSKEIEAKIKELTSTCSPIISKPKPKVEPPKEEQK |
| | | | | NAEQNGPVDGQGDNPGPQAAEQGTDTAVPSDSDKKLPEMDID |
| 11 | 88 | Hsp110 | 814 | MSVVGLDVGSQSCYIAVARAGGIETIANEFSDRCTPSVISFG |
| | | | | SKNRTIGVAAKNQQITHANNTVSNFKRFHGRAFNDPFIQKEK |
| | | | | ENLSYDLVPLKNGGVGIKVMYMGEEHLFSVEQITAMLLTKLK |
| | | | | ETAENSLKKPVTDCVISVPSFFTDAERRSVLDAAQIVGLNCL |
| | | | | RLMNDMTAVALNYGIYKQDLPSLDEKPRIVVFVDMGHSAFQV |
| | | | | SACAFNKGKLKVLGTAFDPELGGKNFDEKLVEHFCAEFKTKY |
| | | | | KLDAKSKIRALLRLYQECEKLKKLMSSNSTDLPLNIECEMND |
| | | | | KDVSGKMNRSQFEELCAELLQKIEVPLYSLLEQTHLKVEDVS |
| | | | | AVEIVGGATRIPAVKERIAKFFGKDISTTLNADEAVARGCAL |
| | | | | QCAILSPAFKVREFSVTDAVPFPISLIWNHDSEDTEGVHEVE |
| | | | | SRNHAAPFSKVLTFLRRGPFELEAFYSDPQGVPYPEAKIGRE |
| | | | | VVQNVSAQKDGEKSRVKVKVRVNTHGIFTISTASMVEKVPTE |
| | | | | ENEMSSEADMECLNQRPPENPDTDANEKKVDQPPEAKKPKIK |
| | | | | VVNVELPIEANLVWQLGKDLLNMYIETEGKMIMQDKLEKERN |
| | | | | DAKNAVEEYVYEFRDKLCGPYEKFICEQDHQNFLRLLTETED |
| | | | | WLYEEGEDQAKQAYVDKLEELMKIGTPVKVRFQEAEERPKMF |
| | | | | EELGQRLQHYAKIAADERNKDEKYNHIDESEMKKVEKSVNEV |
| | | | | MEWMNNVMNAQAKKSLDQDPVVRAQEIKTKIKELNNTCEPVV |
| | | | | TQPKPKIESPKLERTPNGPNIDKKEEDLEDKNNFGAEPPHQN |
| | | | | GECYPNEKNSVNMDLD |
| 12 | 89 | JB1- | 339 | MGKDYYQTLGLARGASDEEIKRAYRRQALRYHPDKNKEPGAE |
| | | scFv(51C1) | | EKFKEIAEAYDVLSDPRKREIFDRYGEEGLKGSDIGGGGSGG |
| | | | | GGSGGGGSGGGGSAAAEVQLVESGGGLVQPGGSLRISCTTSG |
| | | | | FIFSDYWMHWVRQAPGKGLTWVSRINLDGSDTIYADSVKGRE |
| | | | | TISRDNDKNTLYLQMNSLRVEDTAIYYCARSRKSVWGQGTMV |
| | | | | TVSSGGGGSGGGGSGGGGSQSALTQPASVSGSPGQSITISCT |
| | | | | GSNTDVGAYDYVSWSQQLPGKAPKFVIFDVDVRPSGISDRES |
| | | | | GSKSGNTASLTISGLQAEDEADYYCSSYTKSGTLVFGGGTKV |
| | | | | TVV |
| 13 | 90 | JB1- | 348 | MGKDYYQTLGLARGASDEEIKRAYRRQALRYHPDKNKEPGAE |
| | | scFv(3F10) | | EKFKEIAEAYDVLSDPRKREIFDRYGEEGLKGSDIGGGGSGG |
| | | | | GGSGGGGSGGGGSAAAEVQLLESGGDLVQPGGSLRLSCAASG |
| | | | | FTFSSQAMSWVRQAPGKGLEWVSALSRTGDYTWYADSVRGRF |
| | | | | TVSRDDSKNIFYLEMNSLRAEDTAVYYCAKNYYSSFGYNWAA |
| | | | | FHIWGQGTMVTVSSGGGGSGGGGSGGGGSEIVLTQSPGTLSL |
| | | | | SPGERATLSCRASQDVNNNYLAWYQQKPGQAPRLLIYGASRR |
| | | | | ATGVPDRESGRGSGTDFTLTINRLEPEDFAMYFCQQYGGSPP |
| | | | | YTFGQGTKLEIK |
| 14 | 91 | JB1-2XQBP1 | 135 | MGKDYYQTLGLARGASDEEIKRAYRRQALRYHPDKNKEPGAE |
| | | | | EKFKEIAEAYDVLSDPRKREIFDRYGEEGLKGSDIGGGGSGG |
| | | | | GGSGGGGSGGGGSAAASNWKWWPGIFDGLESNWKWWPGIFDS |
| | | | | RDYKDDDDK |
| 15 | 92 | JC7- | 338 | MDYYKILGVDKNASEDEIKKAYRKRALMHHPDRHSGASAEVQ |
| | | scFv(3B12A) | | KEEEKKFKEVGEAFTILSDPKKKTRYDSGQDIGGGGSGGGGS |
| | | | | GGGGSGGGGSAAAEVQLQQSGAELVKPGASVKLSCTASGENI |
| | | | | KDYYMHWVKQRTEQGLEWIGRIDPEDGETKYAPKFQGKATIT |
| | | | | ADTSSNTAYLQLSSLTSEDTAVYYCTIIYYYGSRYVDYWGQG |
| | | | | TTLTVSGGGGSGGGGSGGGGSEIVLTQSPTTMAASPGEKITI |
| | | | | TCSASSSISSSYLHWYQQKPGFSPKLLIYRTSNLASGVPARF |
| | | | | SGSGSGTSYSLTIGTMEAEDVATYYCQQGSSIPLTFGSGTKL |
| | | | | EI |
| 16 | 93 | JB13- | 341 | MGQDYYSVLGITRNSEDAQIKQAYRRLALKHHPLKSNEPSSA |
| | | scFv(3B12A) | | EIFRQIAEAYDVLSDPMKRGIYDKFGEEGLKGGDIGGGGSGG |
| | | | | GGSGGGGSGGGGSAAAEVQLQQSGAELVKPGASVKLSCTASG |
| | | | | FNIKDYYMHWVKQRTEQGLEWIGRIDPEDGETKYAPKFQGKA |
| | | | | TITADTSSNTAYLQLSSLTSEDTAVYYCTIIYYYGSRYVDYW |
| | | | | GQGTTLTVSGGGGSGGGGSGGGGSEIVLTQSPTTMAASPGEK |
| | | | | ITITCSASSSISSSYLHWYQQKPGFSPKLLIYRTSNLASGVP |
| | | | | ARFSGSGSGTSYSLTIGTMEAEDVATYYCQQGSSIPLTFGSG |
| | | | | TKLEI |
| 17 | 94 | JB1- | 321 | MGKDYYQTLGLARGASDEEIKRAYRRQALRYHPDKNKEPGAE |
| | | scFv(3B12A) | | EKFKEIAEAYDVLSDPRKREIFDRYGEEGLKGSGGGGSEVQL |
| | | (1) | | QQSGAELVKPGASVKLSCTASGFNIKDYYMHWVKQRTEQGLE |
| | | | | WIGRIDPEDGETKYAPKFQGKATITADTSSNTAYLQLSSLTS |
| | | | | EDTAVYYCTIIYYYGSRYVDYWGQGTTLTVSGGGGSGGGGSG |
| | | | | GGGSEIVLTQSPTTMAASPGEKITITCSASSSISSSYLHWYQ |
| | | | | QKPGFSPKLLIYRTSNLASGVPARFSGSGSGTSYSLTIGTME |
| | | | | AEDVATYYCQQGSSIPLTFGSGTKLEI |
| 18 | 95 | JB1- | 321 | MGKDYYQTLGLARGASDEEIKRAYRRQALRYHPDKNKEPGAE |
| | | scFv(3B12A) | | EKFKEIAEAYDVLSDPRKREIFDRYGEEGLKGSEAAAKEVQL |
| | | (2) | | QQSGAELVKPGASVKLSCTASGFNIKDYYMHWVKQRTEQGLE |
| | | | | WIGRIDPEDGETKYAPKFQGKATITADTSSNTAYLQLSSLTS |
| | | | | EDTAVYYCTIIYYYGSRYVDYWGQGTTLTVSGGGGSGGGGSG |
| | | | | GGGSEIVLTQSPTTMAASPGEKITITCSASSSISSSYLHWYQ |
| | | | | QKPGFSPKLLIYRTSNLASGVPARFSGSGSGTSYSLTIGTME |
| | | | | AEDVATYYCQQGSSIPLTFGSGTKLEI |
| 19 | 96 | JB1- | 316 | MGKDYYQTLGLARGASDEEIKRAYRRQALRYHPDKNKEPGAE |
| | | scFv(3B12A) | | EKFKEIAEAYDVLSDPRKREIFDRYGEEGLKGSEVQLQQSGA |
| | | (3) | | ELVKPGASVKLSCTASGFNIKDYYMHWVKQRTEQGLEWIGRI |
| | | | | DPEDGETKYAPKFQGKATITADTSSNTAYLQLSSLTSEDTAV |
| | | | | YYCTIIYYYGSRYVDYWGQGTTLTVSGGGGSGGGGSGGGGSE |
| | | | | IVLTQSPTTMAASPGEKITITCSASSSISSSYLHWYQQKPGF |
| | | | | SPKLLIYRTSNLASGVPARFSGSGSGTSYSLTIGTMEAEDVA |
| | | | | TYYCQQGSSIPLTFGSGTKLEI |
| 20 | 97 | JB1- | 497 | MGKDYYQTLGLARGASDEEIKRAYRRQALRYHPDKNKEPGAE |
| | | scFv(3F10)- | | EKFKEIAEAYDVLSDPRKREIFDRYGEEGLKGSDIGGGGSGG |
| | | DD | | GGSGGGGSAAAEVQLQQSGAELVKPGASVKLSCTASGENIKD |
| | | | | YYMHWVKQRTEQGLEWIGRIDPEDGETKYAPKFQGKATITAD |
| | | | | TSSNTAYLQLSSLTSEDTAVYYCTIIYYYGSRYVDYWGQGTT |
| | | | | LTVSGGGGSGGGGSGGGGSEIVLTQSPTTMAASPGEKITITC |
| | | | | SASSSISSSYLHWYQQKPGESPKLLIYRTSNLASGVPARFSG |
| | | | | SGSGTSYSLTIGTMEAEDVATYYCQQGSSIPLTFGSGTKLEI |
| | | | | LEGDYKDDDDKGSRFTRRGEDLFMCMDIQLVEALCGFQKPIS |
| | | | | TLDNRTIVITSHPGQIVKHGDIKCVLNEGMPIYRRPYEKGRL |
| | | | | IIEFKVNFPENGELSPDKLSLLEKLLPERKEVEETDEMDQVE |
| | | | | LVDFDPNQERRRHYNGEAYEDDEHHPRGGVQCQTS |

### II. Nucleic Acids Encoding Fusion Protein Constructs

According to another aspect of the invention, provided are isolated nucleic acids comprising a polynucleotide sequence selected from (a) a polynucleotide encoding the fusion protein of any of the foregoing embodiments, or (b) the complement of the polynucleotide of (a). The present invention provides isolated nucleic acids encoding fusion proteins comprising the J domain and TDP-43-binding domain, and sequences complementary to such nucleic acid molecules encoding the fusion proteins, including homologous variants thereof. In another aspect, the invention encompasses methods to produce nucleic acids encoding the fusion proteins disclosed herein, and sequences complementary to the nucleic acid molecules encoding fusion proteins, including homologous variants thereof. The nucleic acid according to this aspect of the invention can be a pre-messenger RNA (pre-mRNA), messenger RNA (mRNA), RNA, genomic DNA (gDNA), PCR amplified DNA, complementary DNA (cDNA), synthetic DNA, or recombinant DNA.

In yet another aspect, disclosed is a method of producing a fusion protein comprising (a) synthesizing and/or assembling nucleotides encoding the fusion protein, (b) incorporating the encoding gene into an expression vector appropriate for a host cell, (c) transforming the appropriate host cell with the expression vector, and (d) culturing the host cell under conditions causing or permitting the fusion protein to be expressed in the transformed host cell, thereby producing the biologically-active fusion protein, which is recovered as an isolated fusion protein by standard protein purification methods known in the art. Standard recombinant techniques in molecular biology is used to make the polynucleotides and expression vectors of the present invention.

In accordance with the invention, nucleic acid sequences that encode the fusion proteins disclosed herein (or its complement) are used to generate recombinant DNA molecules that direct the expression of the fusion proteins in appropriate host cells. Several cloning strategies are suitable for performing the present invention, many of which is used to generate a construct that comprises a gene coding for a fusion protein of the present invention, or its complement. In some embodiments, the cloning strategy is used to create a gene that encodes a fusion protein of the invention, or their complement.

In certain embodiments, a nucleic acid encoding one or more fusion proteins is an RNA molecule, and can be a pre-messenger RNA (pre-mRNA), messenger RNA (mRNA), RNA, genomic DNA (gDNA), PCR amplified DNA, complementary DNA (cDNA), synthetic DNA, or recombinant DNA.

In various embodiments, the nucleic acid is an mRNA that is introduced into a cell in order to transiently express a desired polypeptide. As used herein, “transient” refers to expression of a non-integrated transgene for a period of hours, days or weeks, wherein the period of time of expression is less than the period of time for expression of the polynucleotide if integrated into the genome or contained within a stable plasmid replicon in the cell.

In particular embodiments, the mRNA encoding a polypeptide is an in vitro transcribed mRNA. As used herein, “in vitro transcribed RNA” refers to RNA, preferably mRNA that has been synthesized in vitro. Generally, the in vitro transcribed RNA is generated from an in vitro transcription vector. The in vitro transcription vector comprises a template that is used to generate the in vitro transcribed RNA.

In particular embodiments, mRNAs may further comprise a comprise a 5′ cap or modified 5′ cap and/or a poly (A) sequence. As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap comprises a terminal group which is linked to the first transcribed nucleotide and recognized by the ribosome and protected from Rnases. The capping moiety can be modified to modulate functionality of mRNA such as its stability or efficiency of translation. In a particular embodiment, the mRNA comprises a poly (A) sequence of between about 50 and about 5000 adenines. In one embodiment, the mRNA comprises a poly (A) sequence of between about 100 and about 1000 bases, between about 200 and about 500 bases, or between about 300 and about 400 bases. In one embodiment, the mRNA comprises a poly (A) sequence of about 65 bases, about 100 bases, about 200 bases, about 300 bases, about 400 bases, about 500 bases, about 600 bases, about 700 bases, about 800 bases, about 900 bases, or about 1000 or more bases. Poly (A) sequences can be modified chemically or enzymatically to modulate mRNA functionality such as localization, stability or efficiency of translation.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms include polynucleotides in which one or more nucleotides have been added or deleted or replaced with different nucleotides compared to a reference polynucleotide. In this regard, it is well understood in the art that certain alterations inclusive of mutations, additions, deletions and substitutions can be made to a reference polynucleotide whereby the altered polynucleotide retains the biological function or activity of the reference polynucleotide.

In certain embodiments, the nucleic acid sequence comprises a nucleotide sequence encoding the gene of interest (e.g., the fusion proteins comprising a J domain and a polyglutamine binding domain) within a nucleic acid cassette. The term “nucleic acid cassette” or “expression cassette” as used herein refers to genetic sequences within the vector which can express an RNA, and subsequently a polypeptide. In one embodiment, the nucleic acid cassette contains a gene(s)-of-interest, e.g., a polynucleotide(s)-of-interest. In another embodiment, the nucleic acid cassette contains one or more expression control sequences, e.g., a promoter, enhancer, poly (A) sequence, and a gene(s)-of-interest, e.g., a polynucleotide(s)-of-interest. Vectors may comprise 1, 2, 3, 4, 5, 6, 7, 8, 9 or 10 or more nucleic acid cassettes. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. The cassette can be removed and inserted into a plasmid or viral vector as a single unit.

Illustrative ubiquitous expression control sequences suitable for use in particular embodiments include, but are not limited to, a cytomegalovirus (CMV) immediate early promoter, a viral simian virus 40 (SV40) (e.g early or late), a Moloney murine leukemia virus (MoMLV) LTR promoter, a Rous sarcoma virus (RSV) LTR, a herpes simplex virus (HSV) (thymidine kinase) promoter, H5, P7.5, and PI I promoters from vaccinia virus, an elongation factor 1-alpha (EFla) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), ß-kinesin (b-KIN), the human ROSA 26 locus (Irions et al, Nature Biotechnology 25, 1477-1482 (2007)), a Ubiquitin C promoter (UBC), a phosphogly cerate kinase-1 (PGK) promoter, a cytomegalovirus enhancer/chicken ß-actin (CAG) promoter (Okabe et al. (1997) FEBS let. 407:313-9), a b-actin promoter and a myeloproliferative sarcoma virus enhancer, negative control region deleted, dl587rev primer binding site substituted (MND) U3 promoter (Haas et al., Journal of Virology. 2003;77 (17): 9439-9450).

In one embodiment, at least one element may be used with the polynucleotides described herein to enhance the transgene target specificity and expression (See e.g., Powell et al. (2015) Discovery Medicine 19 (102): 49-57, the contents of which are herein incorporated by reference in its entirety) such as promoters. Promoters for which promote expression in most tissues include, but are not limited to, human elongation factor la-subunit (EFla), immediate-early cytomegalovirus (CMV), chicken β-actin (CBA) and its derivative CAG, the B glucuronidase (GUSB), or ubiquitin C (UBC). Tissue-specific expression elements can be used to restrict expression to certain cell types such as, but not limited to, nervous system promoters which can be used to restrict expression to neurons, astrocytes, or oligodendrocytes. Non-limiting example of tissue-specific expression elements for neurons include neuron-specific enolase (NSE), platelet-derived growth factor (PDGF), platelet-derived growth factor B-chain (PDGF-β), the synapsin (Syn), the methyl-CpG binding protein 2 (MeCP2), CaMKII, mGluR2, NFL, NFH, nB2, PPE, Enk and EAAT2 promoters. A non-limiting example of a tissue-specific expression elements for astrocytes include the glial fibrillary acidic protein (GFAP) and EAAT2 promoters. A non-limiting example of a tissue-specific expression element for oligodendrocytes include the myelin basic protein (MBP) promoter. Yu et al. (2011) Molecular Pain, 7:63, incorporated by reference in its entirety) evaluated the expression of eGFP under the CAG, EFla, PGK and UBC promoters in rat DRG cells and primary DRG cells using lentiviral vectors and found that UBC showed weaker expression than the other 3 promoters and there was only 10-12% glia expression seen for all promoters. Soderblom et al. (E. Neuro 2015, incorporated by reference in its entirety) the expression of eGFP in AAV8 with CMV and UBC promoters and AAV2 with the CMV promoter after injection in the motor cortex. Intranasal administration of a plasmid containing a UBC or EFla promoter showed a sustained airway expression greater than the expression with the CMV promoter (See e.g., Gill et al, (2001) Gene Therapy, Vol. 8, 1539-1546; incorporated by reference in its entirety). Husain et al. (2009) Gene Therapy, incorporated by reference in its entirety) evaluated a HβH construct with a hGUSB promoter, a HSV-1LAT promoter and a NSE promoter and found that the HβH construct showed weaker expression than NSE in mice brain. Passini and Wolfe (J. Virol. 2001, 12382-12392, incorporated by reference in its entirety) evaluated the long term effects of the HβH vector following an intraventricular injection in neonatal mice and found that there was sustained expression for at least 1 year. Low expression in all brain regions was found by Xu et al. (2001) Gene Therapy, 8, 1323-1332; incorporated by reference in its entirety) when NF-L and NF-H promoters were used as compared to the CMV-lacZ, CMV-luc, EF, GFAP, hENK, nAChR, PPE, PPE+wpre, NSE (0.3 kb), NSE (1.8 kb) and NSE (1.8 kb+wpre). Xu et al. found that the promoter activity in descending order was NSE (1.8 kb), EF, NSE (0.3 kb), GFAP, CMV, hENK, PPE, NFL and NFH. NFL is a 650 nucleotide promoter and NFH is a 920 nucleotide promoter which are both absent in the liver but NFH is abundant in the sensory proprioceptive neurons, brain and spinal cord and NFH is present in the heart. Scn8a is a 470 nucleotide promoter which expresses throughout the DRG, spinal cord and brain with particularly high expression seen in the hippocampal neurons and cerebellar Purkinje cells, cortex, thalamus and hypothalamus (See e.g., Drews et al. 2007 and Raymond et al. 2004; incorporated by reference in its entirety).

### III. Vectors Comprising Nucleic Acids Encoding Fusion Proteins

Also provided is a vector comprising nucleic acid according to the invention. Such a vector preferably comprises additional nucleic acid sequences such as elements necessary for transcription/translation of the nucleic acid sequence encoding a phosphatase (for example promoter and/or terminator sequences). Said vectors can also comprise nucleic acid sequences coding for selection markers (for example an antibiotic) to select or maintain host cells transformed with said vector. The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. In particular embodiments, non-viral vectors are used to deliver one or more polynucleotides contemplated herein to an affected cell (e.g. neuronal cells) In one embodiment, the vector is an in vitro synthesized or synthetically prepared mRNA encoding a fusion protein comprising a J domain and a TDP-43-binding domain. Illustrative examples of non-viral vectors include, but are not limited to mRNA, plasmids (e.g., DNA plasmids or RNA plasmids), transposons, cosmids, and bacterial artificial chromosomes.

Illustrative examples of vectors include, but are not limited to, a plasmid, autonomously replicating sequences, and transposable elements, e.g., piggyBac, Sleeping Beauty, Mosl, Tcl/mariner, Tol2, mini-Tol2, Tc3, MuA, Himar I, Frog Prince, and derivatives thereof. Additional Illustrative examples of vectors include, without limitation, plasmids, phagemids, cosmids, artificial chromosomes such as yeast artificial chromosome (YAC), bacterial artificial chromosome (BAC), or PI-derived artificial chromosome (PAC), bacteriophages such as lambda phage or M13 phage, and animal viruses. Illustrative examples of viruses useful as vectors include, without limitation, retrovirus (including lentivirus), adenovirus, adeno-associated virus, herpesvirus (e.g., herpes simplex vims), poxvirus, baculovirus, papillomavirus, and papovavirus (e.g., SV40). Illustrative examples of expression vectors include, but are not limited to, pClneo vectors (Promega) for expression in mammalian cells; pLenti4/V 5-DEST™, pLenti6/V 5-DEST™, and pLenti6.2/V 5-GW/lacZ (Invitrogen) for lentivirus-mediated gene transfer and expression in mammalian cells. In particular embodiments, coding sequences of polypeptides disclosed herein can be ligated into such expression vectors for the expression of the polypeptides in mammalian cells.

In particular embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally.

The vectors may comprise one or more recombination sites for any of a wide variety of site-specific recombinases. It is to be understood that the target site for a site-specific recombinase is in addition to any site(s) required for integration of a vector, e.g., a retroviral vector or lentiviral vector. As used herein, the terms “recombination sequence,” “recombination site,” or “site specific recombination site” refer to a particular nucleic acid sequence to which a recombinase recognizes and binds.

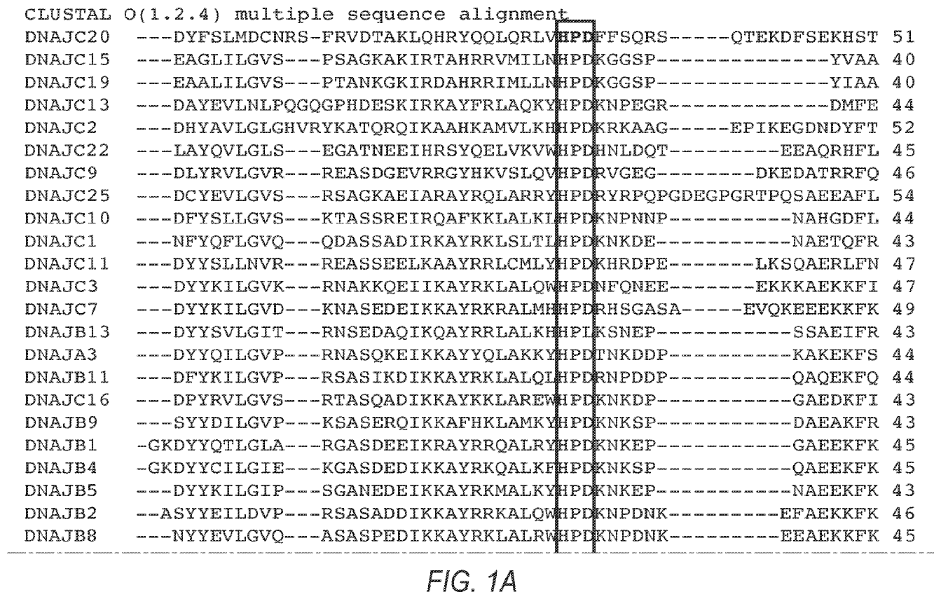

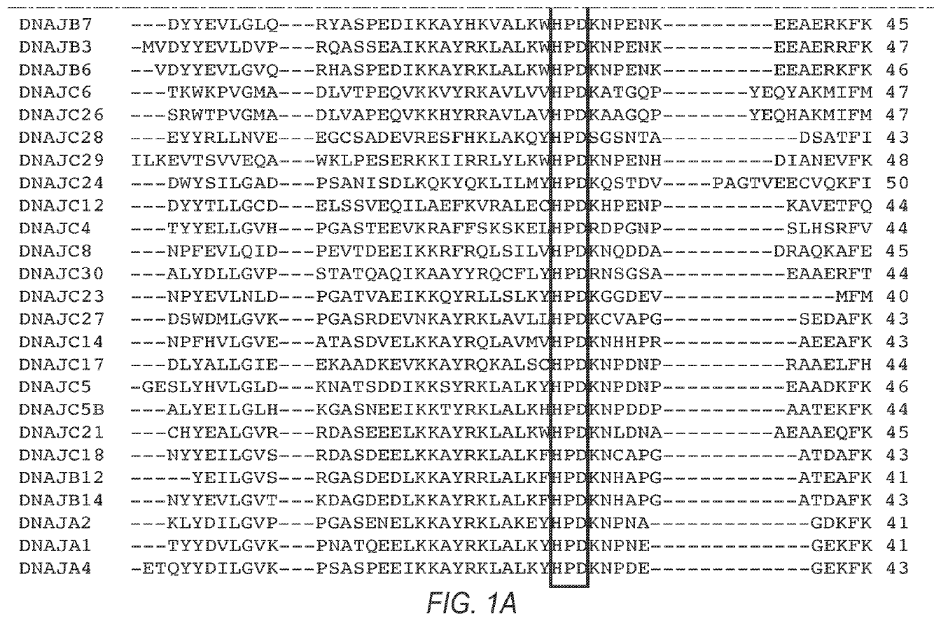

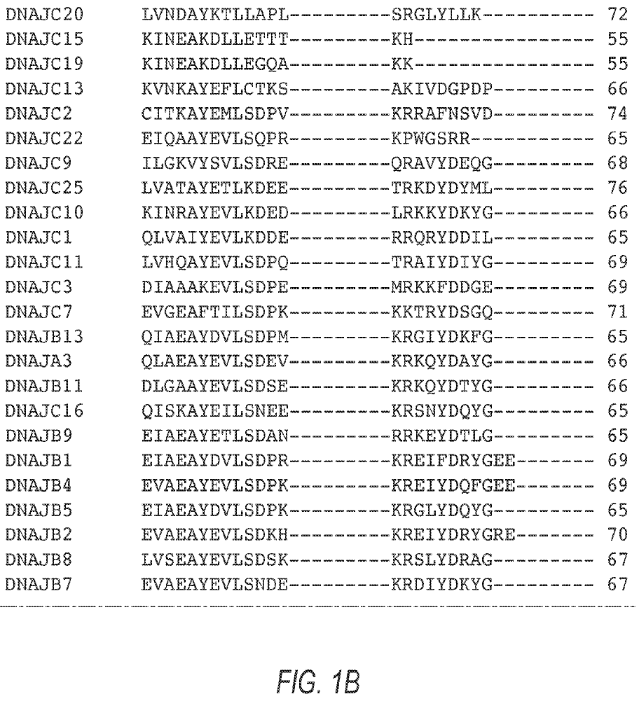

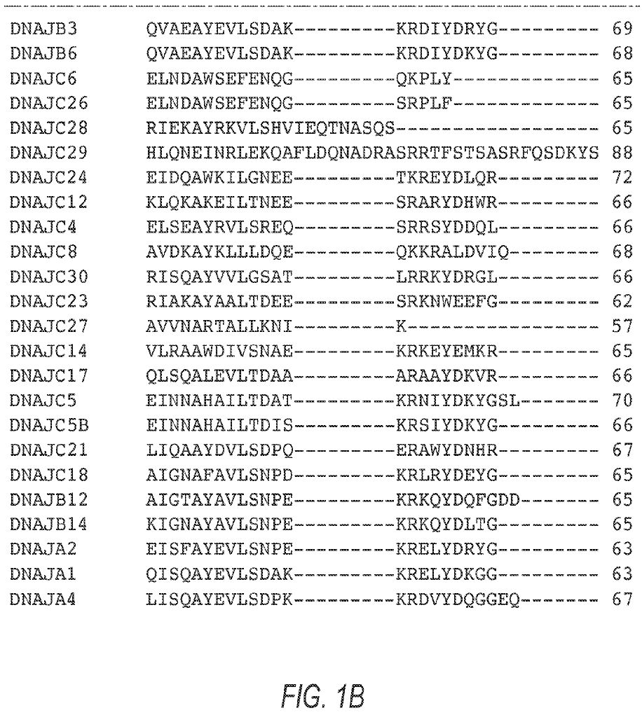

For example, one recombination site for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)). Suitable recognition sites for the FLP recombinase include, but are not limited to: FRT (McLeod, et al., 1996), FI, F2, F3 (Schlake and Bode, 1994), FyFs (Schlake and Bode, 1994), FRT (LE) (Senecoff et al., 1988), FRT (RE) (Senecoff et al., 1988).

Other examples of recognition sequences are the attB, attP, attL, and attR sequences, which are recognized by the recombinase enzyme | Integrase, e.g., phi-c3l. The (pC31 SSR mediates recombination only between the heterotypic sites attB (34 bp in length) and attP (39 bp in length) (Groth et al., 2000). attB and attP, named for the attachment sites for the phage integrase on the bacterial and phage genomes, respectively, both contain imperfect inverted repeats that are likely bound by ϕ031 homodimers (Groth et al., 2000). The product sites, attL and attR, are effectively inert to further tpQA 1-mediated recombination (Belteki et al., 2003), making the reaction irreversible. For catalyzing insertions, it has been found that attB-bearing DNA inserts into a genomic attP site more readily than an attP site into a genomic attB site (Thyagarajan et al., 2001; Belteki et al., 2003). Thus, typical strategies position by homologous recombination an attP-bearing “docking site” into a defined locus, which is then partnered with an attB-bearing incoming sequence for insertion.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15 (12): 477-83) and Jackson and Kaminski. 1995. RNA 1 (10): 985-1000. In particular embodiments, vectors include one or more polynucleotides-of-interest that encode one or more polypeptides. In particular embodiments, to achieve efficient translation of each of the plurality of polypeptides, the polynucleotide sequences can be separated by one or more IRES sequences or polynucleotide sequences encoding self-cleaving polypeptides. In one embodiment, the IRES used in polynucleotides contemplated herein is an EMCV IRES.

As used herein, the term “Kozak sequence” refers to a short nucleotide sequence that greatly facilitates the initial binding of mRNA to the small subunit of the ribosome and increases translation. (Kozak, 1986. Cell. 44 (2): 283-92, and Kozak, 1987. Nucleic Acids Res. 15 (20): 8125-48). In particular embodiments, the vectors comprise polynucleotides that have a consensus Kozak sequence and that encode a fusion protein comprising a J domain and TDP-43-binding domain. Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed.

Illustrative examples of viral vector systems suitable for use in particular embodiments contemplated herein include but are not limited to adeno-associated virus (AAV), retrovirus, herpes simplex virus, adenovirus, and vaccinia virus vectors.

In various embodiments, one or more polynucleotides encoding fusion protein comprising a J domain and a polyglutamine-binding domain are introduced into a cell, e.g., a neuronal cell, by transducing the cell with a recombinant adeno-associated virus (rAAV), comprising the one or more polynucleotides. AAV is a small (˜26 nm) replication-defective, primarily episomal, non-enveloped virus. AAV can infect both dividing and non-dividing cells and may incorporate its genome into that of the host cell. Recombinant AAV (rAAV) are typically composed of, at a minimum, a transgene and its regulatory sequences, and 5′ and 3′ AAV inverted terminal repeats (ITRs). The ITR sequences are about 145 bp in length. In particular embodiments, the rAAV comprises ITRs and capsid sequences isolated from AAV1, AAV2 (described, for example, in U.S. Pat. No. 6,962,815B2, which is incorporated herein by reference in its entirety), AAV3, AAV4, AAV5 (described, for example, in U.S. Pat. No. 7,479,554B2, which is incorporated herein by reference in its entirety), AAV6, AAV7, AAV8 (described, for example, in U.S. Pat. No. 7,282,199B2, which is incorporated herein by reference in its entirety), AAV9 (described, for example, in U.S. Pat. No. 9,737,618B2, which is incorporated herein by reference in its entirety), AAV rh10 (described, for example, in U.S. Pat. No. 9,790,472B2, which is incorporated herein by reference in its entirety) or AAV 10. In one embodiment, the vector of the present invention is encapsulated into a capsid selected from the group consisting of AAV2, AAV5, AAV8, AAV9 and AAV rh10. In one embodiment, the vector is encapsulated in AAV2. In one embodiment, the vector is encapsulated in AAV5. In one embodiment, the vector is encapsulated in AAV8. In one embodiment, the vector is encapsulated in AAV9. In still one embodiment, the vector is encapsulated in AAV rh10.

In some embodiments, a chimeric rAAV is used the ITR sequences are isolated from one AAV serotype and the capsid sequences are isolated from a different AAV serotype. For example, a rAAV with ITR sequences derived from AAV2 and capsid sequences derived from AAV6 is referred to as AAV2/AAV6. In particular embodiments, the rAAV vector may comprise ITRs from AAV2, and capsid proteins from any one of AAV1, AAV2, AAV3, AAV4, AAV5, AAV6, AAV7, AAV8, AAV9, or AAV10. In a preferred embodiment, the rAAV comprises ITR sequences derived from AAV2 and capsid sequences derived from AAV6. In a preferred embodiment, the rAAV comprises ITR sequences derived from AAV2 and capsid sequences derived from AAV2.

In some embodiments, engineering and selection methods can be applied to AAV capsids to make them more likely to transduce cells of interest.

Construction of rAAV vectors, production, and purification thereof have been disclosed, e.g., in U.S. Pat. Nos. 9,169,494; 9,169,492; 9,012,224; 8,889,641; 8,809,058; and 8,784,799, each of which is incorporated by reference herein, in its entirety.

### IV. Delivery

In particular embodiments, one or more polynucleotides encoding a fusion protein comprising a J domain and TDP-43-binding domain are introduced into a cell by non-viral or viral vectors. Illustrative methods of non-viral delivery of polynucleotides contemplated in particular embodiments include, but are not limited to: electroporation, sonoporation, lipofection, microinjection, biolistics, virosomes, liposomes, immunoliposomes, nanoparticles, poly cation or lipidnucleic acid conjugates, naked DNA, artificial virions, DEAE-dextran-mediated transfer, gene gun, and heat-shock.

Illustrative examples of polynucleotide delivery systems suitable for use in particular embodiments contemplated in particular embodiments include, but are not limited to those provided by Amaxa Biosystems, Maxcyte, Inc., BTX Molecular Delivery Systems, and Copernicus Therapeutics Inc. Lipofection reagents are sold commercially (e.g., Transfectam™ and Lipofectin™). Cationic and neutral lipids that are suitable for efficient receptor-recognition lipofection of polynucleotides have been described in the literature. See e.g., Liu et al., (2003) Gene Therapy. 10:180-187; and Balazs et al., (20W) Journal of Drug Delivery. 2011:1-12. Antibody-targeted, bacterially derived, non-living nanocell-based delivery is also contemplated in particular embodiments.

Viral vectors comprising polynucleotides contemplated in particular embodiments can be delivered in vivo by administration to an individual patient, typically by systemic administration (e.g., intravenous, intraperitoneal, intramuscular, subdermal, or intracranial infusion), by intrathecal injection, intracerebroventricular injection or topical application, as described below. Alternatively, vectors can be delivered to cells ex vivo, such as cells explanted from an individual patient (e.g., mobilized peripheral blood, lymphocytes, bone marrow aspirates, tissue biopsy, etc.) or universal donor hematopoietic stem cells, followed by reimplantation of the cells into a patient.

In one embodiment, a viral vector comprising a polynucleotide encoding a fusion protein disclosed herein is administered directly to an organism for transduction of cells in vivo.

A viral vector, suitably packaged and formulated, can be delivered into the central nervous system (CNS) via intrathecal delivery. For example, adeno-associated viral vectors can be delivered using methods described in U.S. Ser. No. 15/771,481, which is incorporated herein by reference in its entirety.

Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

In various embodiments, one or more polynucleotides encoding a fusion protein disclosed herein are introduced into a cell, for example, a neuronal cell or neuronal stem cell, by transducing the cell with a retrovirus, e.g., lentivirus, comprising the one or more polynucleotides. As used herein, the term “retrovirus” refers to an RNA virus that reverse transcribes its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus. As used herein, the term “lentivirus” refers to a group (or genus) of complex retroviruses. Illustrative lentiviruses include, but are not limited to: HIV (human immunodeficiency virus; including HIV type 1, and HIV 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred.

Lentiviral vectors preferably contain several safety enhancements as a result of modifying the LTRs. “Self-inactivating” (SIN) vectors refers to replication-defective vectors, e.g., in which the right (3′) LTR enhancer-promoter region, known as the U3 region, has been modified (e.g., by deletion or substitution) to prevent viral transcription beyond the first round of viral replication. An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex vims (HSV) (thymidine kinase) promoters. In certain embodiments, lentiviral vectors are produced according to known methods. See e.g., Kutner et al., BMC Biotechnol. 2009; 9:10. Doi: 10.1186/1472-6750-9-10; Kutner et al., Nat. Protoc. 2009; 4 (4): 495-505. Doi: 10.1038/nprot.2009.22.

According to certain specific embodiments contemplated herein, most or all of the viral vector backbone sequences are derived from a lentivirus, e.g., HIV-I. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. Moreover, a variety of lentiviral vectors are known in the art, see Naldini et al., (1996a, 1996b, and 1998); Zufferey et al., (1997); Dull et al., 1998, U.S. Pat. Nos. 6,013,516; and 5,994,136, many of which may be adapted to produce a viral vector or transfer plasmid contemplated herein.

In various embodiments, one or more polynucleotides encoding a fusion protein disclosed herein are introduced into a target cell by transducing the cell with an adenovirus comprising the one or more polynucleotides. Adenoviral based vectors are capable of very high transduction efficiency in many cell types and do not require cell division. With such vectors, high titer and high levels of expression have been obtained. This vector can be produced in large quantities in a relatively simple system. Most adenovirus vectors are engineered such that a transgene replaces the Ad Ela, Elb, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity.

Generation and propagation of the current adenovirus vectors, which are replication deficient, may utilize a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones & Shenk, 1978), the current adenovirus vectors, with the help of 293 cells, carry foreign DNA in either the E1, the D3 or both regions (Graham & Prevec, 1991). Adenovirus vectors have been used in eukaryotic gene expression (Levrero et al., 1991; Gomez-Foix et al., 1992) and vaccine development (Grunhaus & Horwitz, 1992; Graham & Prevec, 1992). Studies in administering recombinant adenovirus to different tissues include trachea instillation (Rosenfeld et al., 1991; Rosenfeld et al., 1992), muscle injection (Ragot et al., 1993), peripheral intravenous injections (Herz & Gerard, 1993) and stereotactic inoculation into the brain (Le Gal La Salle et al., 1993). An example of the use of an Ad vector in a clinical trial involved polynucleotide therapy for antitumor immunization with intramuscular injection (Sterman et al., Hum. Gene Ther. 7:1083-9 (1998)).

In various embodiments, one or more polynucleotides encoding a fusion protein of the invention are introduced into the target cell of a subject by transducing the cell with a herpes simplex virus, e.g., HSV—I, HSV-2, comprising the one or more polynucleotides.

The mature HSV virion consists of an enveloped icosahedral capsid with a viral genome consisting of a linear double-stranded DNA molecule that is 152 kb. In one embodiment, the HSV based viral vector is deficient in one or more essential or non-essential HSV genes. In one embodiment, the HSV based viral vector is replication deficient. Most replication deficient HSV vectors contain a deletion to remove one or more intermediate-early, early, or late HSV genes to prevent replication. For example, the HSV vector may be deficient in an immediate early gene selected from the group consisting of: ICP4, ICP22, ICP27, ICP47, and a combination thereof. Advantages of the HSV vector are its ability to enter a latent stage that can result in long-term DNA expression and its large viral DNA genome that can accommodate exogenous DNA inserts of up to 25 kb. HSV-based vectors are described in, for example, U.S. Pat. Nos. 5,837,532, 5,846,782, and 5,804,413, and International Patent Applications WO 91/02788, WO 96/04394, WO 98/15637, and WO 99/06583, each of which is incorporated by reference herein in its entirety.

### V. Cells Expressing the Fusion Protein

In yet another aspect, the invention provides for cells expressing the fusion proteins described herein. Cells can be transfected with a vector encoding the fusion protein as described herein above. In one embodiment, the cell is a prokaryotic cell. In another embodiment, the cell is a eukaryotic cell. In still another embodiment, the cell is a mammalian cell. In a particular embodiment, the cell is a human cell. In another embodiment, the cell is a human cell that is derived from a patient that suffers from, or is at risk of suffering from, a TDP-43-mediated disorder including, but not limited to, ALS, FTD and Alzheimer's Disease. The cell can be a neuronal cell or a muscle cell.

Cells expressing the fusion protein can be useful in producing the fusion protein. In this embodiment, the cells are transfected with a vector overexpressing the fusion protein. The fusion protein may optionally contain an epitope, for example, a human Fc domain or a FLAG epitope, as described herein above, that would facilitate the purification (using a Protein A- or anti-FLAG antibody column, respectively). The epitope may be connected to the rest of the fusion protein via a linker or a protease substrate sequence such that, during or after purification, the epitope can be removed from the fusion protein.

Cells expressing the fusion protein can also be useful in a therapeutic context. In one embodiment, cells are collected from a patient in need of therapy (e.g., a patient who suffers from or is at risk of suffering from a TDP-43-mediated disorder). In one embodiment, the cells are neuronal cells. Collected cells are then transfected with a vector expressing the fusion protein. The transfected cells can then be processed to enrich or select for transfected cells. The transfected cells can also be treated to differentiate into a different type of cell, for example, a neuronal cell. After processing, the transfected cells can be administered to the patient. In one embodiment, the cells are administered by directed injection into the central nervous system by intrathecal injection, intracranial injection or intracerebroventricular injection.

In an alternative embodiment, cells expressing a secreted form of the fusion protein can be used. For example, fusion protein constructs can be designed having a signal sequence on the N-terminal end. Representative signal sequences are shown below in Table 7.

TABLE 7
Representative Signal Sequences

| SEQ ID NO: | SEQUENCE |
|---|---|
| 98 | MGVKVLFALICIAVAEA |
| 99 | MAPVOLLGLLVLELPAMRC |
| 100 | MAVLGLLFCLVTFPSCVLS |

Therefore, in one embodiment, the fusion protein comprises a signal sequence and a fusion protein, wherein the signal sequence is selected from the group consisting of SEQ ID NOs: 98-100, and a fusion protein comprising a J domain and a TDP-43 binding domain. In one embodiment, the the signal sequence is selected from the group consisting of SEQ ID NOS: 98-100, and a fusion protein is selected from the group consisting of SEQ ID NOs: 80-85, 89-90 and 92-96. In another embodiment, the fusion protein comprises the signal sequence of SEQ ID NO: 98, and the fusion protein selected from the group consisting of SEQ ID NOs: 80-85, 89-90 and 92-96. In another embodiment, the fusion protein comprises the signal sequence of SEQ ID NO: 99, and the fusion protein selected from the group consisting of SEQ ID NOs: 80-85, 89-90 and 92-96. In another embodiment, the fusion protein comprises the signal sequence of SEQ ID NO: 100, and the fusion protein selected from the group consisting of SEQ ID NOs: 80-85, 89-90 and 92-96. Cells expressing the fusion protein constructs with the signal sequence can be administered to a subject, for example a human subject (e.g., a patient having or at risk of suffering from a TDP-43 disorder). The fusion protein is secreted from the cells, which help reduce TDP-43 protein aggregation and/or associated cytotoxicity.

As described herein above, in certain embodiments, the fusion protein can further comprise a cell-penetrating peptide. A cell expressing a fusion protein comprising a signal sequence and a cell-penetrating peptide would be capable of secreting the fusion protein, devoid of the signal sequence. The secreted fusion protein, also comprising the cell-penetrating peptide, would then be capable of entering nearby cells, and have the potential to reduce aggregation and/or cytotoxicity mediated by TDP-43 proteins in those cells.

### VI. Methods of Use

In another aspect, the invention provides a method for achieving a beneficial effect in disorders and/or in a TDP-43 disorder, disorder or condition mediated by TDP-43 aggregation. The TDP-43 disorder is selected from the group consisting of amyotrophic lateral sclerosis (ALS), frontotemporal dementia (FTD), Parkinson's disease, Huntington's disease, Alzheimer's disease, hippocampal sclerosis, dementia with Lewy's bodies, and limbic predominant age-related TDP-43 encephalopathy.

In some embodiments, the invention provides methods for treating a subject, such as a human, with a TDP-43 disease, disorder or condition comprising the step of administering to the subject a therapeutically- or prophylactically-effective amount of a fusion protein, a nucleic acid encoding such fusion protein, or a viral vector encoding such fusion protein described herein, wherein said administration results in the improvement of one or more biochemical or physiological parameters or clinical endpoints associated with the TDP-43 disease, disorder or condition.

In other embodiments, the invention provides for a method of reducing aggregation of TDP-43 in a cell. The cell can be a cultured cell or an isolated cell. The cell can also be from a subject, for example, a human subject. In one embodiment, the cell is in the central nervous system of the human subject. In another embodiment, the human subject is suffering from, or is at risk of suffering from a TDP-43 disorder disease, including, but not limited to, amyotrophic lateral sclerosis (ALS), frontotemporal dementia (FTD) and Alzheimer's Disease. In one particular embodiment, the TDP-43 disorder is amyotrophic lateral sclerosis.

Aggregation of TDP-43 proteins can be detected in a number of ways. In one example, aggregated TDP-43 proteins can be distinguished from free (i.e., soluble) TDP-43 containing proteins based on solubility, for example, by trapping the insoluble aggregates by passage of cellular lysates through a selective filter. Non-aggregated proteins pass through these filters, whereas aggregates will be retained on the filter, which can be detected using any number of reagents, including antibodies directed against the TDP-43 protein. The amount of trapped aggregated protein in the lysate of a cell sample treated with the fusion protein, cell expressing the fusion protein, or a nucleic acid, vector, or viral particle encoding the fusion protein as described herein can be compared with lysates from an untreated or control-treated cell, where a reduction in the amount of aggregated TDP-43 protein in the treated sample when compared with the control sample is indicative of efficacy of the fusion protein or a nucleic acid, vector, or viral particle encoding the fusion protein (see, for example, Kim et al., (2014) Mol. Cell. Biol., 34:643-652, and Example 1). A greater reduction in the aggregated TDP-43 protein, when compared with controls indicates a higher potency. Reduction of aggregation of TDP-43 proteins can also be detected directly in the cell, for example, using immunofluorescence microscopy with labeled reagents detecting the TDP-43 protein (see, for example, Ding et al., (2015) Oncotarget, 6:24178-24191; Chou et al., (2015) Hum. Mol. Genet. 24:5154-5173, and Example 1). In certain embodiments, a greater reduction of TDP-43 polypeptide levels when compared with controls indicates a higher potency.

Therefore, in one embodiment, the method comprises contacting the cell with an amount of the fusion protein or a nucleic acid, vector, or viral particle encoding the fusion protein effective to reduce aggregation of TDP-43 proteins by at least 10%, for example, at least 15%, at least 20%, at least 30%, at least 40%, at least 50%, at least 60%, at least 70%, at least 75%, at least 80%, at least 90%, at least 95%, at least 98%, at least 99%, when compared with an untreated or control cell.

As shown below in Example 1, expression of fusion proteins comprising a J domain and a TDP-43 binding domain has been found to reduce the overall level of TDP-43-containing reporter constructs. As such, in another embodiment, the method comprises contacting the cell with an amount of the fusion protein, a cell expressing the fusion protein, a nucleic acid, vector, or viral particle encoding the fusion protein effective to reduce the level of TDP-43 proteins by at least 10%, for example, at least 15%, at least 20%, at least 30%, at least 40%, at least 50%, at least 60%, at least 70%, at least 75%, at least 80%, at least 90%, at least 95%, at least 98%, at least 99%, when compared with an untreated or control cell.

### VII. Pharmaceutical Compositions

The compositions contemplated herein may comprise one or more fusion protein comprising a J domain and TDP-43-binding domain, polynucleotides encoding such fusion proteins, vectors comprising same, genetically modified cells, etc., as contemplated herein. Compositions include, but are not limited to pharmaceutical compositions. A “pharmaceutical composition” refers to a composition formulated in pharmaceutically acceptable or physiologically acceptable solutions for administration to a cell or an animal, either alone, or in combination with one or more other modalities of therapy. It will also be understood that, if desired, the compositions may be administered in combination with other agents as well, such as, e.g., cytokines, growth factors, hormones, small molecules, chemotherapeutics, pro-drugs, drugs, antibodies, or other various pharmaceutically active agents. There is virtually no limit to other components that may also be included in the compositions, provided that the additional agents do not adversely affect the ability of the composition to deliver the intended therapy.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier”, “diluent” or “excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

### VIII. Dosages

The dosage of the compositions (e.g., a composition including a fusion protein construct, nucleic acid or gene therapy viral particle) described herein, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. The compositions described herein can be administered initially in a suitable dosage that can be adjusted as required, depending on the clinical response. In some aspects, the dosage of a composition is a prophylactically or a therapeutically effective amount.

### IX. Kits

Kits including (a) a pharmaceutical composition including a fusion protein construct, nucleic acid encoding such fusion protein, or viral particle encompassing such nucleic acid that reduces aggregation of TDP-43 proteins in a cell or subject described herein, and (b) a package insert with instructions to perform any of the methods described herein are contemplated. In some aspects, the kit includes (a) a pharmaceutical composition including a composition described herein that reduces the aggregation of TDP-43 proteins in a cell or subject described herein, (b) an additional therapeutic agent, and (c) a package insert with instructions to perform any of the methods described herein.

### EXAMPLES

To test whether J domains can be specifically engineered to facilitate the proper folding of aggregated proteins, we designed and tested a number of fusion protein constructs designed to target TDP-43 proteins.

### Example 1: Fusion Protein Design

### A. Methods

### General Techniques and Materials

The practice of the present invention employs, unless otherwise indicated, conventional techniques of immunology, biochemistry, chemistry, molecular biology, microbiology, cell biology, genomics and recombinant DNA, which are within the skill of the art. See Sambrook, J. et al., “Molecular Cloning: A Laboratory Manual,” 3^{rd} edition, Cold Spring Harbor Laboratory Press, 2001; “Current protocols in molecular biology”, F. M. Ausubel, et al., eds., 1987; the series “Methods in Enzymology,” Academic Press, San Diego, Calif.; “PCR 2: a practical approach”, M. J. MacPherson, B. D. Hames and G. R. Taylor eds., Oxford University Press, 1995; “Antibodies, a laboratory manual” Harlow, E. and Lane, D. eds., Cold Spring Harbor Laboratory, 1988; “Goodman & Gilman's The Pharmacological Basis of Therapeutics,” 11th Edition, McGraw-Hill, 2005; and Freshney, R. I., “Culture of Animal Cells: A Manual of Basic Technique,” 4th edition, John Wiley & Sons, Somerset, N J, 2000, the contents of which are incorporated in their entirety herein by reference. HEK-293 cells (human embryonic kidney cells) were purchased from the American Type Culture Collection (Manassas, VA). Anti-FLAG antibody was purchased from Thermo Fisher Scientific. Rabbit anti-GFP antibody was purchased from GenScripts (Piscataway, NJ). For ease of purification and characterization, some of the fusion protein constructs used in this Example 1 contain, in addition to the sequences provided in SEQ ID NOs: 80-85 and 89-96, the FLAG epitope of SEQ ID NO:68 at either the C-terminus or N-terminus of the protein, in addition to a short linker sequence.

### Expression and Detection of Proteins in HEK293 Cells

Expression vector plasmids encoding various protein constructs were transfected into HEK293 cells with Lipofectamine 3000 transfection reagent (Thermo Fisher Scientific). Cell lysates were analyzed for expressed proteins using immunoblot assays. Samples of culture media were centrifuged to remove debris prior to analysis. Cells were lysed in a lysis buffer (10 mM Tris-HCl, pH 8.0, 150 mM NaCl, 10 mM EDTA, 2% SDS) containing 2 mM PMSF and protease cocktail (Complete Protease Inhibitor Cocktail; Sigma). After brief sonication, the samples were analyzed for expressed proteins using immunoblot assays. For immunoblot analysis, samples were boiled in an SDS-sample buffer and run on polyacrylamide electrophoresis. Thereafter, the separated protein bands were transferred to a PVDF membrane.

Expressed proteins were detected using a chemiluminescent signal. Briefly, blots were reacted with a primary antibody capable of binding the particular epitope (e.g., GFP). After rinsing away the unreacted primary antibody, a secondary, enzyme-linked antibody (e.g., HRP-linked anti-lgG antibody) was allowed to react with the primary antibody molecules bound to the blots. Following rinsing, a chemiluminescent reagent was added, and the resultant chemiluminescent signals in the blots were captured on X-ray film.

### Fluorescence Microscopy

In some instances, aggregation of TDP-43 (full-length of C-terminal fragment) GFP reporter constructs (described below) were detected in vivo using fluorescence microscopy. Cultured cells expressing the reporter constructs as well as the fusion protein comprising the J domain and TDP-43-binding domain were washed with PBS and fixed with 4% paraformaldehyde in PBS for 5 minutes. After three 5-min washes with PBS, nuclear DNA was stained with DAPI. Percentage of cells containing TDP-43 (GFP foci) in transfected cells was counted.

### Fractionation Assay

Transfected HEK293 cells are homogenized in RIPA buffer (50 mM Tris, pH7.5, 150 mM NaCl, 0.1% NP-40, 0.5% sodium deoxycholate, 0.1% SDS) supplemented with protease inhibitor cocktail, 2 mM PMSF, 10 mM NaF, and 2 mM Na3VO4. After brief sonication, protein concentration is measured by BCA assay kit (Pierce). The equal protein amount is spun down by centrifugation at 16,000×g for 30 minutes at 4° C. to fractionate into soluble fraction (supernatant) and insoluble fraction (pellet). The insoluble fraction is further solubilized in SDS lysis buffer (10 mM Tris, pH8.0, 150 mM NaCl, 2% SDS). Both soluble and insoluble fractions were applied to SDS-PAGE under reducing condition, followed by immunoblotting assay with anti-GFP antibody.

### B. Reporter Constructs

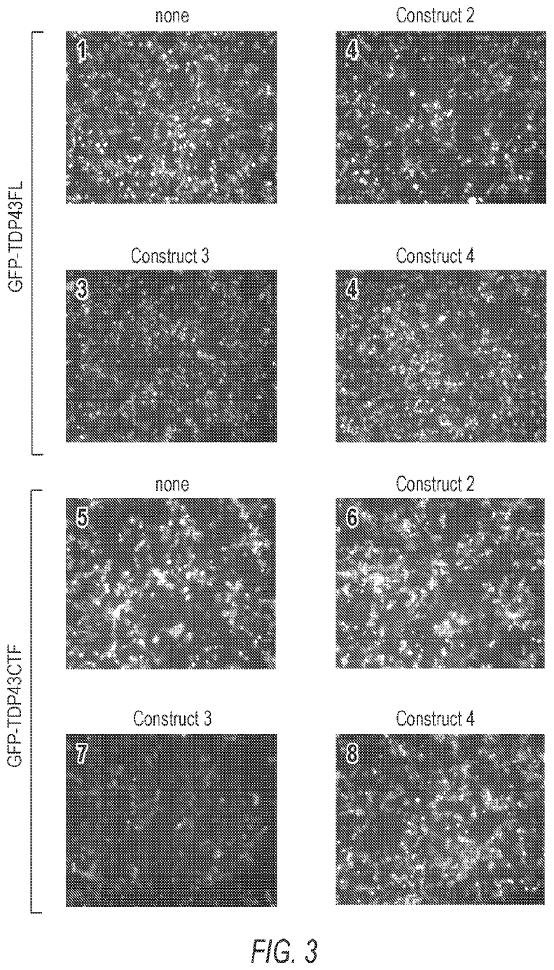

We first investigated whether the fusion molecule of the present invention targeting TDP-43 ameliorates its aggregation in cultured cells. To this end, we generated GFP-based reporter constructs GFP-TDP43 and GFP-TDP43, wherein GFP was fused at its C-terminus to either the full-length human TDP-43 protein or the C-terminal fragment TDP-43 which is known to form cytoplasmic aggregation and cytotoxicity (See Table 8 below). HEK293 cells were cultured and transfected with the plasmids encoding the full-length TDP43 or the C-terminal fragment of TDP43, containing the C-terminal 207 amino acids TDP-43 (amino acids 208-414 of human TDP-43) as a GFP fusion [GFP-TDP43FL (SEQ ID NO: 101) or GFP-TDP43CTF (SEQ ID NO: 102)]. We found that the majority of expressed GFP-TDP43FL was localized in the nucleus of the cell (FIG. 3, panel 1) while GFP-TDP43CTF made conspicuous cytoplasmic inclusions (FIG. 3, panel 5) as previously reported (Zhang et al., (2009) Proc Natl Acad Sci U SA., 106 (18): 7607-12).

TABLE 8
TDP-43 Reporter Constructs

| Construct | SEQ ID | | |
|---|---|---|---|
| Name | NO: | Length | Sequence |
| GFP- | 101 | 660 | MVSKGEELFTGVVPILVELDGDVNGHKFSVSGEGEGDATYG |
| TDP43FL | | | KLTLKFICTTGKLPVPWPTLVTTLTYGVQCFSRYPDHMKQH |
| | | | DFFKSAMPEGYVQERTIFFKDDGNYKTRAEVKFEGDTLVNR |
| | | | IELKGIDFKEDGNILGHKLEYNYNSHNVYIMADKQKNGIKV |
| | | | NFKIRHNIEDGSVQLADHYQQNTPIGDGPVLLPDNHYLSTQ |
| | | | SALSKDPNEKRDHMVLLEFVTAAGITLGMDELYKEFDIAAA |
| | | | MSEYIRVTEDENDEPIEIPSEDDGTVLLSTVTAQFPGACGL |
| | | | RYRNPVSQCMRGVRLVEGILHAPDAGWGNLVYVVNYPKDNK |
| | | | RKMDETDASSAVKVKRAVQKTSDLIVLGLPWKTTEQDLKEY |
| | | | FSTFGEVLMVQVKKDLKTGHSKGFGFVRFTEYETQVKVMSQ |
| | | | RHMIDGRWCDCKLPNSKQSQDEPLRSRKVFVGRCTEDMTED |
| | | | ELREFFSQYGDVMDVFIPKPFRAFAFVTFADDQIAQSLCGE |
| | | | DLIIKGISVHISNAEPKHNSNRQLERSGREGGNPGGFGNQG |
| | | | GFGNSRGGGAGLGNNQGSNMGGGMNFGAFSINPAMMAAAQA |
| | | | ALQSSWGMMGMLASQQNQSGPSGNNQNQGNMQREPNQAFGS |
| | | | GNNSYSGSNSGAAIGWGSASNAGSGSGFNGGFGSSMDSKSS |
| | | | GWGM |
| GFP- | 102 | 453 | MVSKGEELFTGVVPILVELDGDVNGHKFSVSGEGEGDATYG |
| TDP43CTF | | | KLTLKFICTTGKLPVPWPTLVTTLTYGVQCFSRYPDHMKQH |
| | | | DFFKSAMPEGYVQERTIFFKDDGNYKTRAEVKFEGDTLVNR |
| | | | IELKGIDFKEDGNILGHKLEYNYNSHNVYIMADKQKNGIKV |
| | | | NFKIRHNIEDGSVQLADHYQQNTPIGDGPVLLPDNHYLSTQ |
| | | | SALSKDPNEKRDHMVLLEFVTAAGITLGMDELYKEFDIAAA |
| | | | REFFSQYGDVMDVFIPKPFRAFAFVTFADDQIAQSLCGEDL |
| | | | IIKGISVHISNAEPKHNSNRQLERSGREGGNPGGFGNQGGF |
| | | | GNSRGGGAGLGNNQGSNMGGGMNFGAFSINPAMMAAAQAAL |
| | | | QSSWGMMGMLASQQNQSGPSGNNQNQGNMQREPNQAFGSGN |
| | | | NSYSGSNSGAAIGWGSASNAGSGSGENGGFGSSMDSKSSGW |
| | | | GM |
| TDP43FL | 103 | 415 | MSEYIRVTEDENDEPIEIPSEDDGTVLLSTVTAQFPGACGL |
| | | | RYRNPVSQCMRGVRLVEGILHAPDAGWGNLVYVVNYPKDNK |
| | | | RKMDETDASSAVKVKRAVQKTSDLIVLGLPWKTTEQDLKEY |
| | | | FSTFGEVLMVQVKKDLKTGHSKGFGFVRFTEYETQVKVMSQ |
| | | | RHMIDGRWCDCKLPNSKQSQDEPLRSRKVFVGRCTEDMTED |
| | | | ELREFFSQYGDVMDVFIPKPFRAFAFVTFADDQIAQSLCGE |
| | | | DLIIKGISVHISNAEPKHNSNRQLERSGREGGNPGGFGNQG |
| | | | GFGNSRGGGAGLGNNQGSNMGGGMNFGAFSINPAMMAAAQA |
| | | | ALQSSWGMMGMLASQQNQSGPSGNNQNQGNMQREPNQAFGS |
| | | | GNNSYSGSNSGAAIGWGSASNAGSGSGENGGFGSSMDSKSS |
| | | | GWGM* |
| TDP43ANLS | 104 | 415 | MSEYIRVTEDENDEPIEIPSEDDGTVLLSTVTAQFPGACGL |
| | | | RYRNPVSQCMRGVRLVEGILHAPDAGWGNLVYVVNYPKDNA |
| | | | AAMDETDASSAVKVKRAVQKTSDLIVLGLPWKTTEQDLKEY |
| | | | FSTFGEVLMVQVKKDLKTGHSKGFGFVRFTEYETQVKVMSQ |
| | | | RHMIDGRWCDCKLPNSKQSQDEPLRSRKVFVGRCTEDMTED |
| | | | ELREFFSQYGDVMDVFIPKPFRAFAFVTFADDQIAQSLCGE |
| | | | DLIIKGISVHISNAEPKHNSNRQLERSGREGGNPGGFGNQG |
| | | | GFGNSRGGGAGLGNNQGSNMGGGMNFGAFSINPAMMAAAQA |
| | | | ALQSSWGMMGMLASQQNQSGPSGNNQNQGNMQREPNQAFGS |
| | | | GNNSYSGSNSGAAIGWGSASNAGSGSGENGGFGSSMDSKSS |
| | | | GWGM* |

### C. Fusion Protein Constructs

To determine whether the fusion proteins of the present invention could be used to reduce TDP-43 aggregation, an initial experiment was first conducted by co-expression of a fusion protein comprising a J-domain sequence derived from a human Hsp40 J-domain protein, conjugated to the single-chain variable fragment (scFv) that recognizes GFP (data not shown). Surprisingly, when GFP-TDP43CTF was expressed with this construct, most of the aggregation disappeared, whereas no significant effect was observed when GFP scFv (no J-domain sequence) was expressed with GFP-TDP43CTF. This suggested that TDP-43 aggregation could be resolved using the HSP70-mediated pathway (not shown).

We then designed a series of fusion protein constructs, as depicted in Table 9.

TABLE 9
Fusion Protein Constructs and Controls

| Construct | SEQ ID | | |
|---|---|---|---|
| No | NO: | Construct Name | Notes |
| 1 | 78 | J-domain only | Control, containing the J domain from |
| | | | human DnaJB1 |
| 2 | 79 | scFv(3B12A) | scFv(3B12A)-only control; binds TDP-43 |
| 3 | 80 | JB1-scFv(3B12A) | DnaJB1 J domain fused to scFv(3B12A) |
| 4 | 81 | JB1(P33Q)-scFv(3B12A) | Same as construct 3, with a P33Q mutation |
| | | | in the conserved HPD motif |
| 5 | 82 | scFv(3B12A)-JB1 | Reversed arrangement of J domain and |
| | | | TDP-43 from Construct 3 |
| 6 | 83 | scFv(3B12A)-JB1- | J domain sandwiched between two scFv |
| | | scFv(3B12A) | that bind TDP-43 |
| 7 | 84 | JB6-scFv(3B12A) | J domain from DnaJB6 |
| 8 | 85 | JC6-scFv(3B12A) | J domain from DnaJC6 |
| 9 | 86 | DnaJB1 | Full-length J protein control |
| 10 | 87 | Hsp70 | HSP70 control |
| 11 | 88 | Hsp110 | HSP110 control |
| 12 | 89 | JB1-scFv(51C1) | J domain from DnaJB1 fused to alternate |
| | | | scFV that binds TDP-43 |
| 13 | 90 | JB1-scFv(3F10) | J domain from DnaJB1 fused to alternate |
| | | | scFV that binds TDP-43 |
| 14 | 91 | JB1-2XQBP1 | J domain from DnaJB1 fused to two |
| | | | tandem copies of QBP1 |

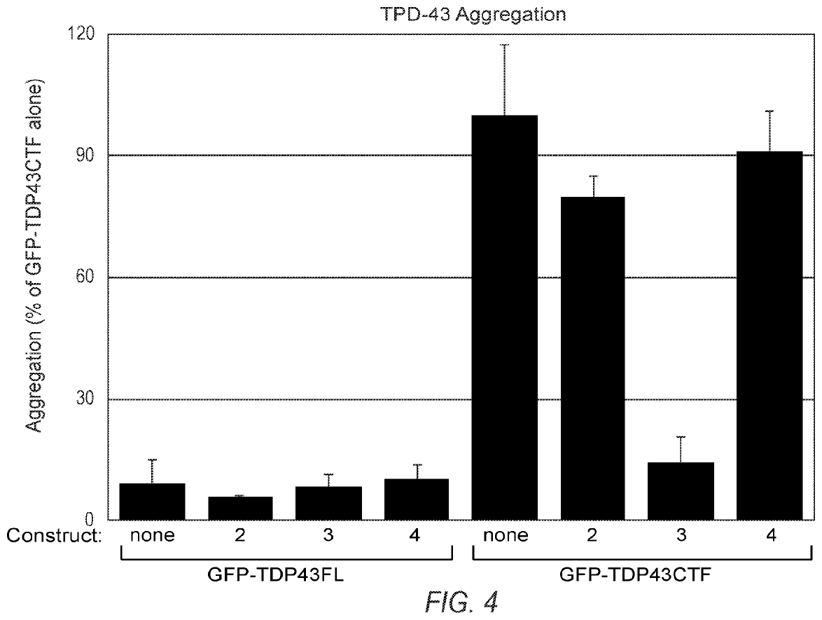

Initial experiments were conducted to test the ability of the fusion protein constructs in reducing TDP-43 aggregation. Construct 3 (JB1-scFv (3B12A)) and companion controls Construct 2 (TDP-43-binding domain only) and Construct 4 (same as Construct 3 but containing a mutation P33Q within the conserved HPD motif of the J domain), and transfected into cells expressing either the GFP-TDP43FL or GFP-TDP43CTF reporter constructs. FIG. 3 shows aggregation of GFP constructs in the cells expressing the GFP-TDP43CTF reporter is greater than in cells expressing GFP-TDP43FL. Cells expressing Construct 3 showed substantially reduced aggregation, whereas expression of the controls (Construct 2 and Construct 4) did not reduce aggregation (FIG. 4; see also Table 10 below). The absence of activity in Construct 4, containing the P33Q, strongly suggests that the ability to reduce aggregation is driven by the J domain acting through the Hsp70 pathway.

TABLE 10
Efficacy of Fusion Protein Constructs in Reducing Aggregation

| | | | % Aggregation |
|---|---|---|---|
| | Construct | | (normalized to GFP- |
| Reporter | No | Construct Name | TDP43CTF alone) |
| GFP-TDP43FL | None | | 9.07% |
| GFP-TDP43FL | 2 | scFv(3B12A) | 5.86% |
| GFP-TDP43FL | 3 | JB1-scFv(3B12A) | 8.54% |
| GFP-TDP43FL | 4 | JB1(P33Q)- | 10.56% |
| | | scFv(3B12A) | |
| GFP-TDP43CTF | None | | 100% |
| GFP-TDP43CTF | 2 | scFv(3B12A) | 80.05% |
| GFP-TDP43CTF | 3 | JB1-scFv(3B12A) | 14.58% |
| GFP-TDP43CTF | 4 | JB1(P33Q)- | 91.25% |
| | | scFv(3B12A) | |

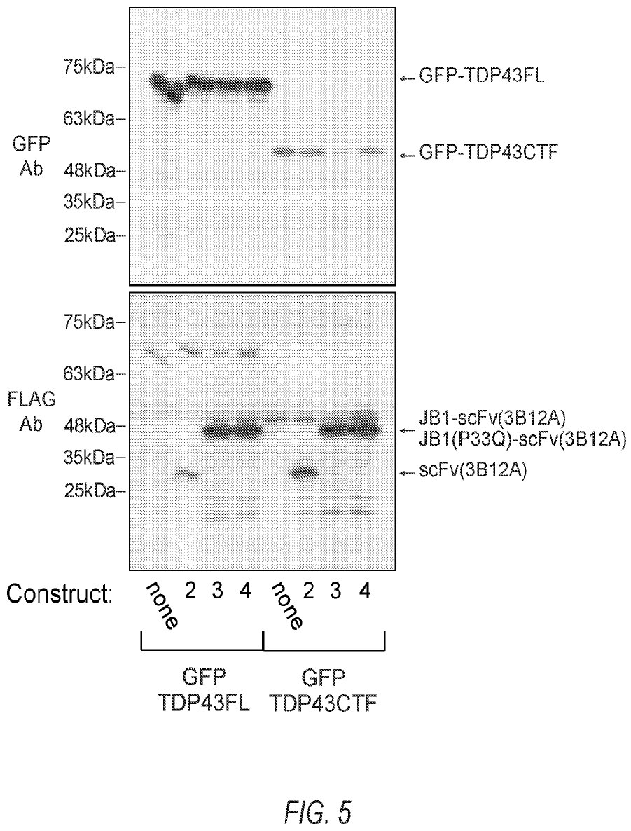

Cellular extracts of these cells were analyzed using immunoblots, using either anti-GFP or anti-FLAG antibodies to determine the level of the GFP-containing reporter construct or the FLAG epitope-containing fusion proteins, respectively (FIG. 5). Cell extracts expressing the GFP-TDP43FL show the presence of a prominent ˜70 kDa band when probed with anti-GFP antibodies, whereas cells expressing GFP-TDP43CTF show the presence of a ˜ 50 kDa band. Interestingly, cells harboring the GFP-TDP43CTF reporter and also expressing construct 3 (JB1-scFv (3B12A)) show a marked reduction in the amount of reporter protein, when compared with the negative control, scFv control (construct 2), or the P33Q mutant (construct 4). No differences in the levels of the full-length reporter construct (GFP-TDP43FL) were seen.

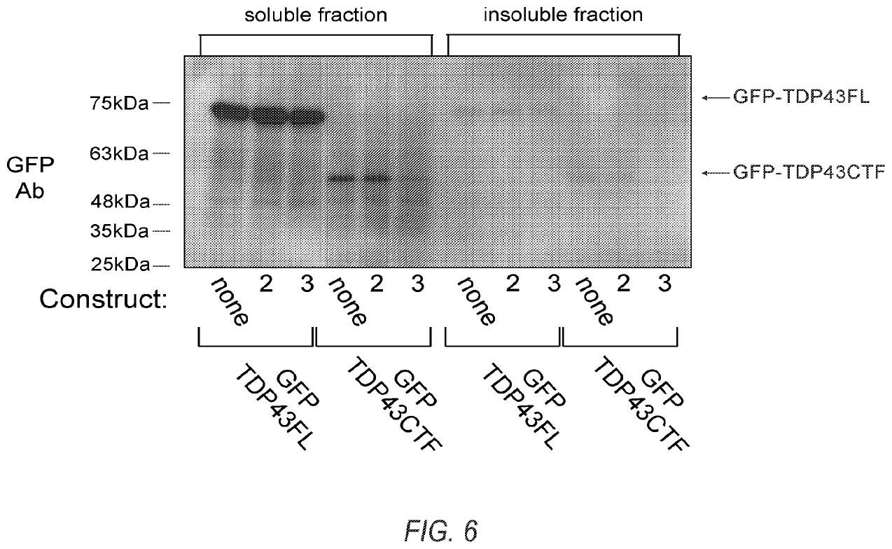

To investigate whether the reduction in TDP-43 levels were dependent on protein aggregation, extracts of cells expressing either the GFP-TDP43FL or GFP-TDP43CTF and also expressing constructs 2 or 3 were fractionated into soluble (unaggregated) and insoluble (aggregated) fractions and detected for the presence of the reporter by probing with anti-GFP antibody. As shown in FIG. 6, in cells expressing the full-length reporter (GFP-TDP43FL), there is no significant change in the level of the reporter in both the soluble and insoluble fractions. In contrast, in cells expressing the GFP-TDP43CTF reporter, a modest decrease in reporter was seen in the soluble fraction of cells expressing construct 3 (JB1-scFv (3B12A)), but not in cells expressing construct 2 (scFv (3B12A)). In contrast, there is significant reduction in the level of reporter an almost complete disappearance of the reporter in the insoluble fraction (presumably the aggregated form).

Taken together, these data strongly suggest that the fusion protein is able to reduce the level of TDP-43 in cells, and may act to preferentially accelerate the clearance of aggregated TDP43 protein.

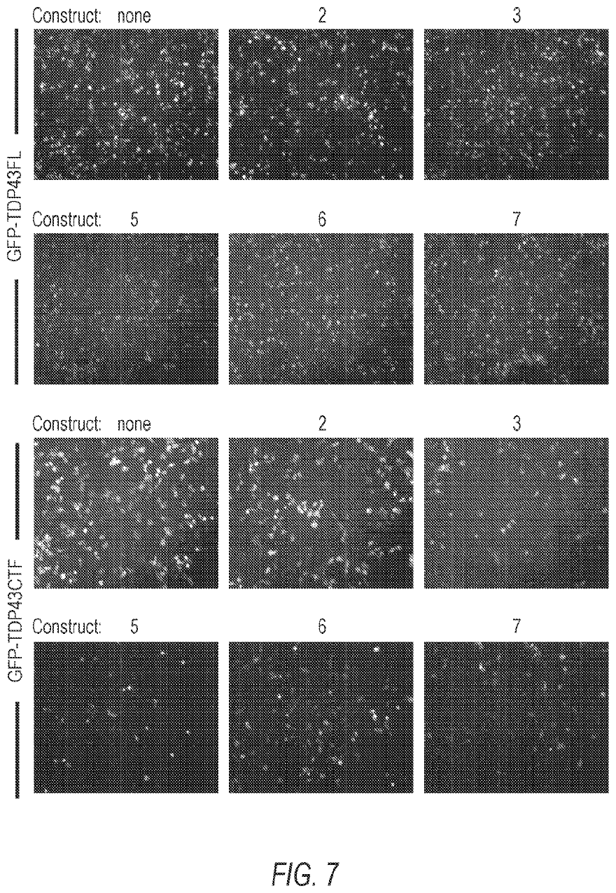

Based on the results above, several additional constructs (Constructs 5-7) were generated containing different configurations of the TDP-43-binding domain in relation to the J domain. These constructs were tested for their ability to reduce aggregation. These new constructs were compared with construct 3 (JB1-scFv (3B12A)), as well as cells expressing none (negative control) or scFv alone (construct 2). FIG. 7 shows results of those experiments (also summarized in Table 11 below).

TABLE 11
Additional Constructs and Controls

| Reporter | Construct No | Construct Name |
|---|---|---|
| GFP-TDP43FL | None | |
| GFP-TDP43FL | 2 | scFv(3B12A) |
| GFP-TDP43FL | 3 | JB1-scFv(3B12A) |
| GFP-TDP43FL | 5 | scFv(3B12A)-JB1 |
| GFP-TDP43FL | 6 | scFv(3B12A)-JB1-scFv(3B12A) |
| GFP-TDP43FL | 7 | JB6-scFv(3B12A) |
| GFP-TDP43CTF | None | |
| GFP-TDP43CTF | 2 | scFv(3B12A) |
| GFP-TDP43CTF | 3 | JB1-scFv(3B12A) |
| GFP-TDP43CTF | 5 | scFv(3B12A)-JB1 |
| GFP-TDP43CTF | 6 | scFv(3B12A)-JB1-scFv(3B12A) |
| GFP-TDP43CTF | 7 | JB6-scFv(3B12A) |

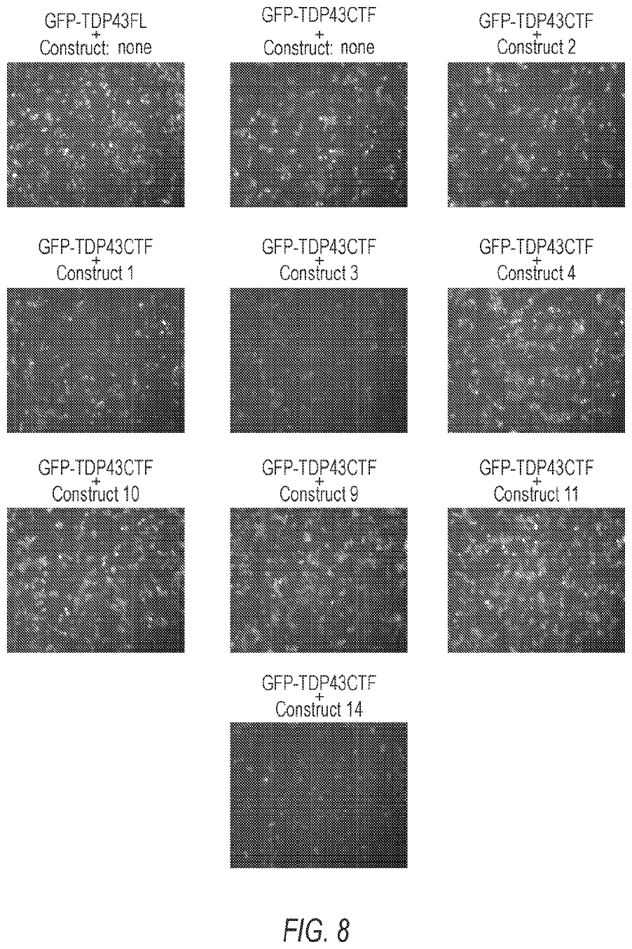

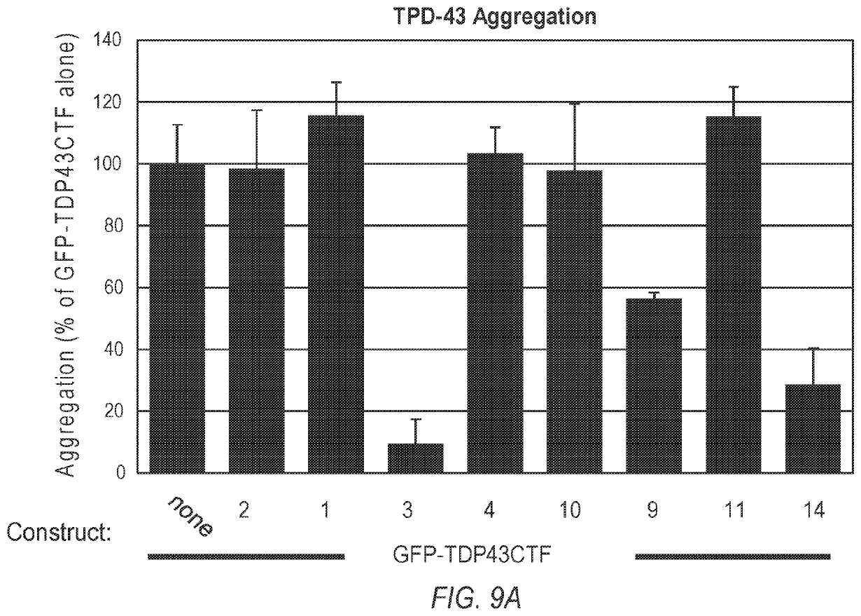

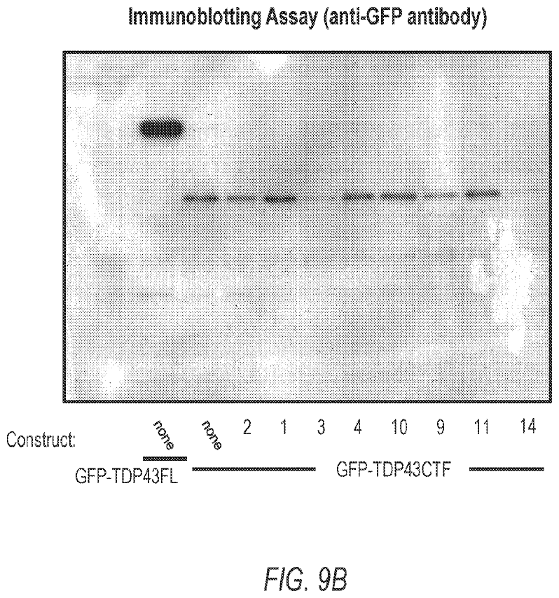

As can be seen in FIG. 7, construct 3 (JB1-scFv (3B12A)), construct 5 (scFv (3B12A)-JB1), construct 6 (scFv (3B12A)-JB1-scFv (3B12A)) and construct 7 (JB6-scFv (3B12A)) show a modest reduction in the aggregation level in cells expressing the GFP-TDP43FL reporter construct when compared with negative controls and cells expressing construct 2 (scFv alone). In contrast, in cells expressing the GFP-TDP43CTF construct, there is a greater overall level of protein aggregation in negative controls and cells expressing construct 2, consistent with earlier observations. Furthermore, cells also expressing construct 3, construct 5, construct 6 and construct 7 all had dramatically reduced levels of protein aggregation, demonstrating that the following configurations of the fusion protein were effective: DNAJ-T, T-DNAJ, T-DNAJ-T (where DNAJ is the J domain, and T is the TDP-43 binding domain). Furthermore, multiple J domains (e.g., from DnaJB1 and DnaJB6) were found to be active in the fusion protein. Additional constructs were then generated and tested, as shown in FIGS. 8 and 9 (see also Table 12 below). Interestingly, expression of the full-length DnaJB1 without a TDP-43 binding domain was able to reduce TDP-43 aggregation by ˜43%, compared with >90% reduction by Construct 3 (JB1-scFv (3B12A)). In addition, expression of Construct 14, which contains two tandem copies of the QBP1 peptide also showed substantial reduction in aggregation (˜71% reduction). QBP1 was previously shown to interact with TDP-43 (see, for example, Mompean et al., (2019) Arch. Biochem. Biophys. 675:108113). Therefore, multiple TDP-43 binding domains (scFv (3B12A) and QBP1) were found to be active when placed in the fusion protein. Furthermore, when cell extracts were analyzed by immunblotting using anti-GFP antibody to quantitate the level of the reporter construct (FIG. 9B), cells expressing Construct 3 (JB1-scFv (3B12A)), Construct 9 (Full-length DnaJB1), and Construct 14 (JB1-2XQBP1) were found to have lower levels of the reporter construct.

TABLE 12
Generation and testing of additional fusion protein constructs

| | | | % Aggregation |
|---|---|---|---|
| | Construct | Construct | (normalized to GFP- |
| Reporter | No | Name | TDP43CTF alone) |
| GFP-TDP43FL | None | | |
| GFP-TDP43CTF | None | | 100% |
| GFP-TDP43CTF | 2 | scFv(3B12A) | 98.40% |
| GFP-TDP43CTF | 1 | JB1-Domain Only | 115.66% |
| GFP-TDP43CTF | 3 | JB1-scFv(3B12A) | 9.29% |
| GFP-TDP43CTF | 4 | JB1(p33Q)- | 103.40% |
| | | scFv(3B12A) | |
| GFP-TDP43CTF | 10 | Hsc70 Control | 97.96% |
| GFP-TDP43CTF | 9 | Full-length | 56.31% |
| | | DnaJB1 | |
| GFP-TDP43CTF | 11 | Hsp110 | 115.34% |
| GFP-TDP43CTF | 14 | JB1-2XQBP1 | 28.55% |

Additional constructs were tested, as shown in Tables 13 and 14 below.

TABLE 13
Generation and testing of additional fusion protein constructs

| | | | | % Phospho-TDP43 |
|---|---|---|---|---|
| | | | | density (normalized |
| | | | | to GFP-TDP43CTF |
| Reporter | Construct No | Construct Name | SEQ ID NO: | alone) |
| GFP-TDP43FL | None | | | 3.36% |
| GFP-TDP43CTF | None | | | 100.00% |
| GFP-TDP43CTF | 3 | JB1-scFv(3B12A) | 80 | 51.84% |
| GFP-TDP43CTF | 7 | JB6-scFv(3B12A) | 84 | 42.21% |
| GFP-TDP43CTF | 2 | scFv(3B12A) | 79 | 122.81% |
| GFP-TDP43CTF | 15 | JC7-scFv(3B12A) | 92 | 47.89% |
| GFP-TDP43CTF | 16 | JB13-scFv(3B12A) | 93 | 106.49% |

TABLE 14
Generation and testing of additional fusion protein constructs

| | | | | % Phospho-TDP43 density | |
|---|---|---|---|---|---|
| | | | | (normalized to GFP- | |
| | | | | TDP43CTF alone) | |
| | Construct | | SEQ ID | Soluble | Insoluble |
| Reporter | No | Construct Name | NO: | fraction | fraction |
| GFP-TDP43FL | None | | | 30.00% | 68.63% |
| GFP-TDP43CTF | None | | | 100.00% | 100.00% |
| GFP-TDP43CTF | 3 | JB1-scFv(3B12A) | 80 | 7.91% | 92.59% |
| GFP-TDP43CTF | 17 | JB1-scFv(3B12A) (1) | 94 | 9.80% | 66.34% |
| GFP-TDP43CTF | 18 | JB1-scFv(3B12A) (2) | 95 | 9.93% | 23.49% |
| GFP-TDP43CTF | 19 | JB1-scFv(3B12A) (3) | 96 | 11.20% | 22.77% |

As shown above, numerous constructs, including those using alternative J domains (see for example, JB6 and JC7 domains) were effective in reducing the GFP-TDP43CTF aggregations. Other fusion protein constructs indicated that J domains from DNAJC6, and the J domain from SV40 or bacterial J-domain protein (DnaJ), were also found to be effective in reducing aggregation of other reporter constructs (data not shown). However, Construct 16, which comprises a J domain without the consensus HPD sequence (See Table 1, SEQ ID NO: 16), was not able to reduce aggregation of the GFP-TDP43CTF reporter construct.

Additional constructs were tested, as shown below in Table 15. Construct 13 (JB1-scFv (3F10), SEQ ID NO: 90), which uses the scFv 3F10 that binds to TDP-43, was fused to the J domain of DNAJB1. Construct 20 (JB1-scFv (3B12A)-DD), SEQ ID NO: 97, containing the dimerization domain from human DnaJA1. As shown below, Construct 13 showed modest ability to reduce aggregation of the GFP-TDP43CTF. Expression of Construct 20, Construct 3 containing the dimerization domain, showed potent reduction of GFP-TDP43CTF aggregation. The further enhancement of the effect by dimerization domain is probably due to the enhanced interaction between the dimerized Construct 3 and the GFP-TDP43CTF, consistent with the domain configuration found in some natural J-domain proteins (Sha (2000) Structure 8 (8), 799-807).

TABLE 15
Generation and testing of additional fusion protein constructs

| | | | % TDP43 density (normalized | |
|---|---|---|---|---|
| | | | to GFP-TDP43CTF alone) | |
| | | | Soluble | Insoluble |
| Reporter | Construct No | Construct Name | fraction | fraction |
| GFP-TDP43FL | None | | 191.46% | 90.99% |
| GFP-TDP43CTF | None | | 100.00% | 100.00% |
| GFP-TDP43CTF | 3 | JB1-scFv(3B12A) | 24.46% | 38.15% |
| GFP-TDP43CTF | 13 | JB1-scFv(3F10) | 43.90% | 61.58% |
| GFP-TDP43CTF | 20 | JB1-scFv(3B12A)-DD | 12.41% | 14.41% |

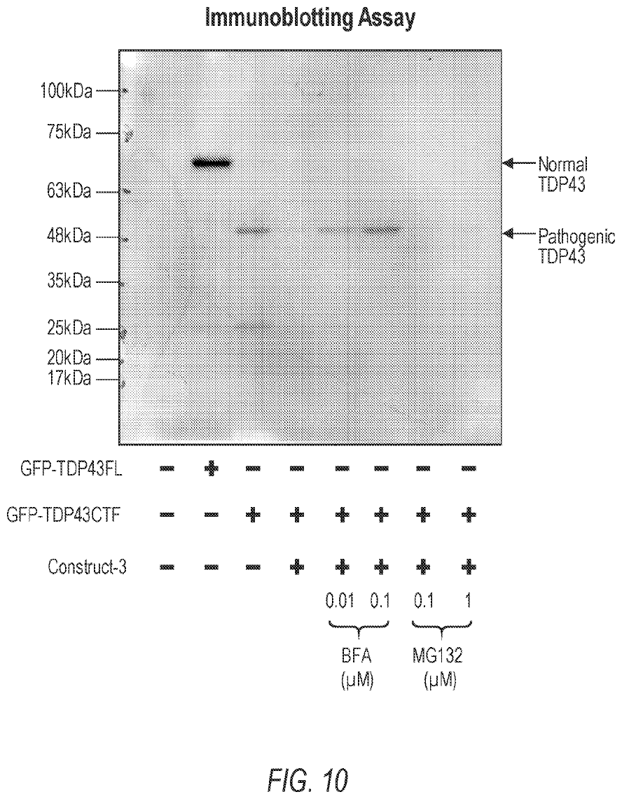

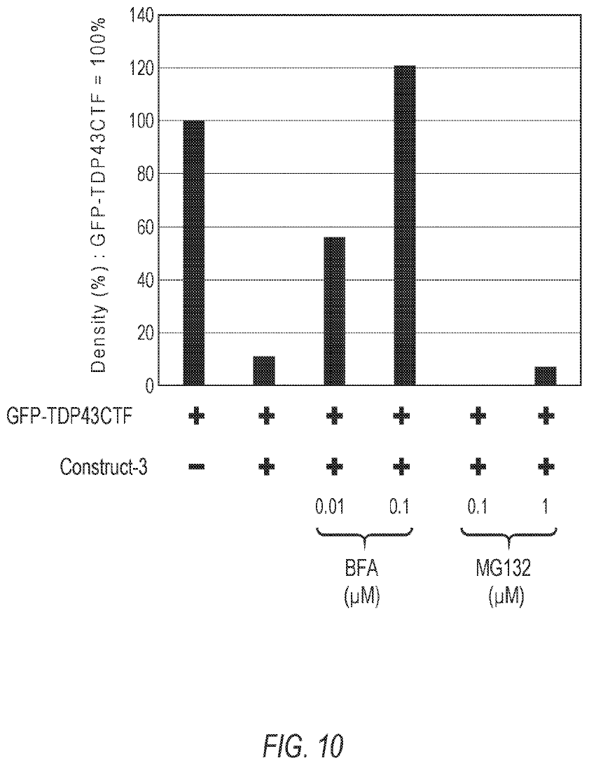

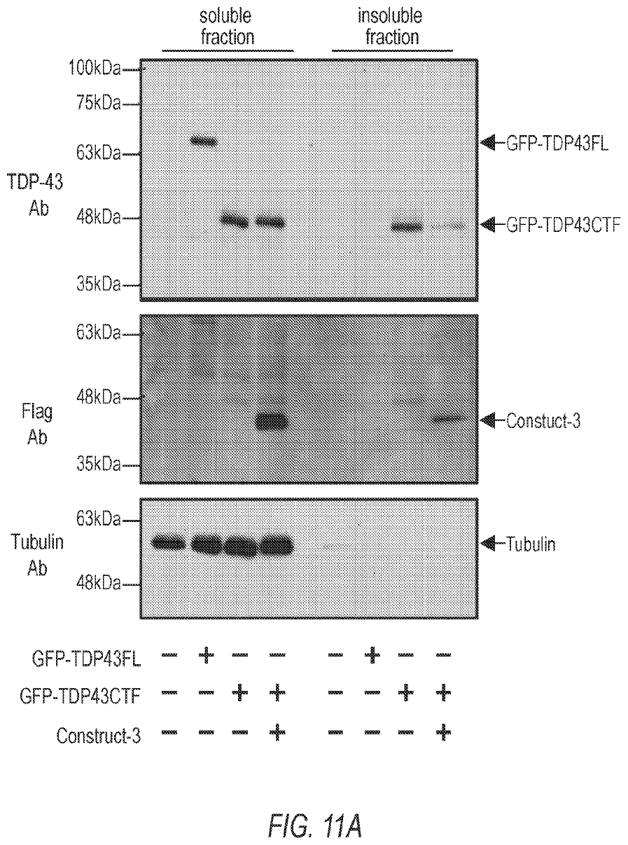

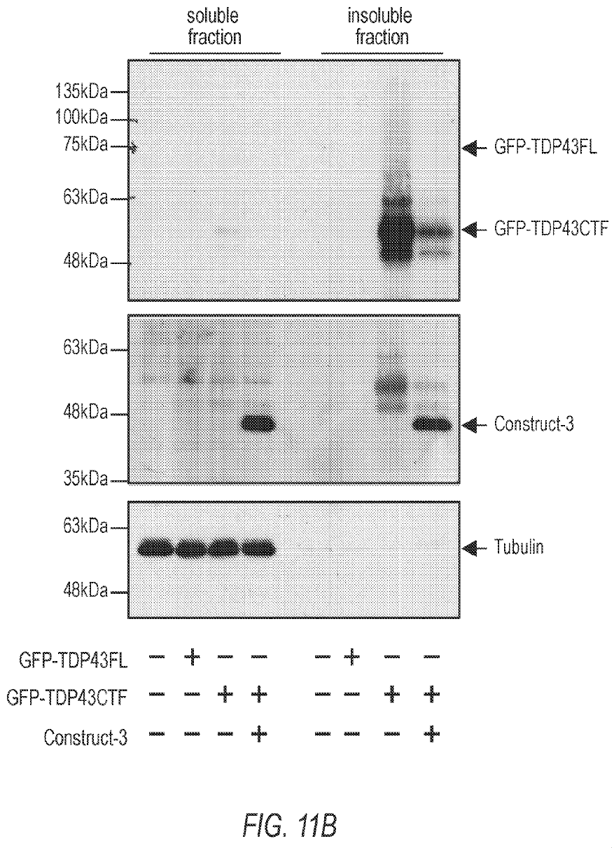

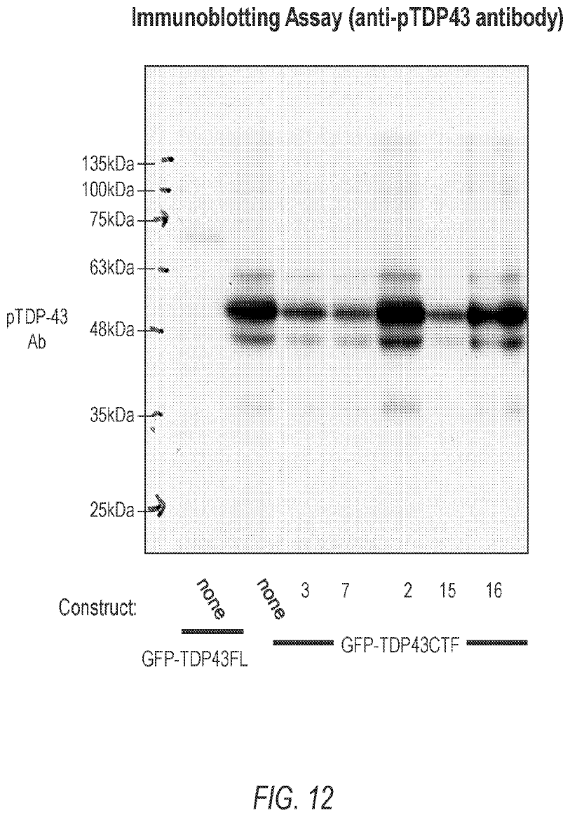

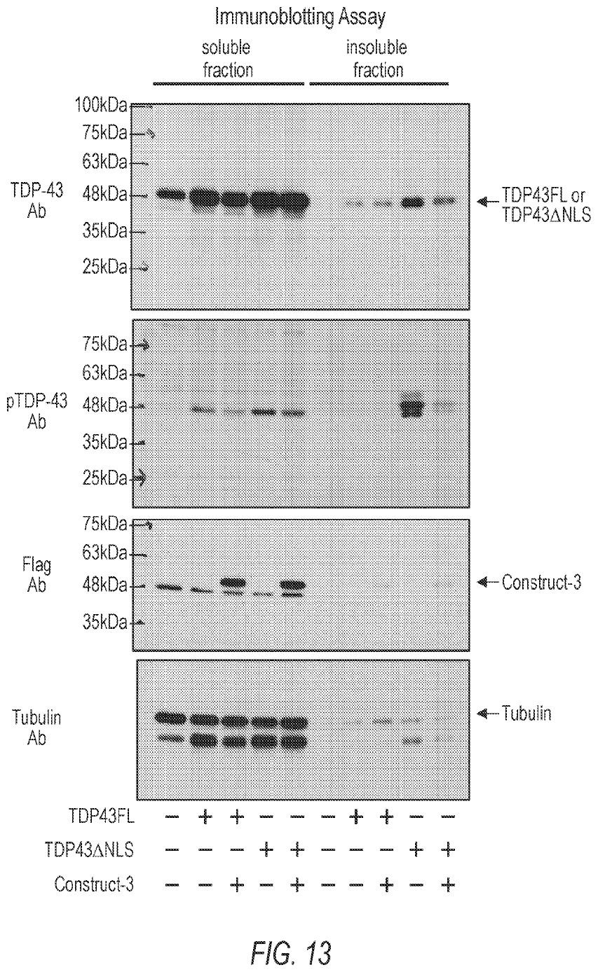

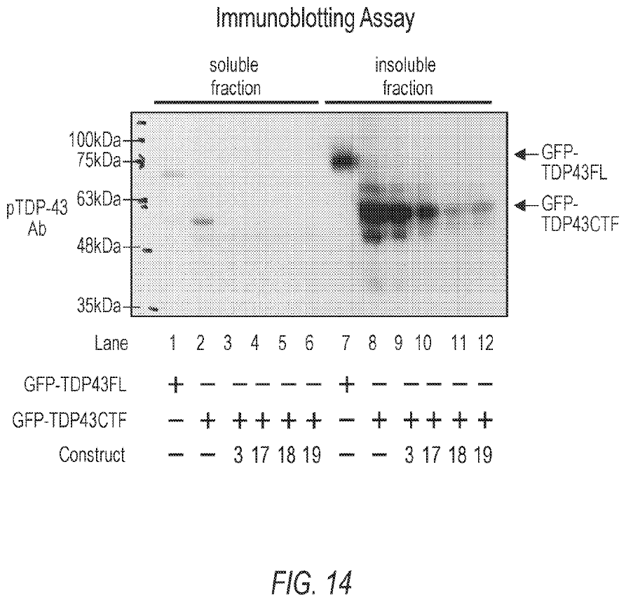

We next investigated the mechanism of the fusion protein in reducing aggregation. HEK293 cells were transfected with the GFP-TDP43FL or GFP-TDP43CTF reporter construct, either alone or with Construct 3 (JB1-scFv (3B12A)), as well as with BFA (Bafilomycin A1, an inhibitor of the late phase of autophagy) or MG132 (proteasome inhibitor). As shown in FIG. 10, treatment of cells with 10 nM or 100 nM BFA resulted in the reappearance of pathogenic TDP43 in a dose dependent manner. In contrast, treatment with 0.1 μM or 1.0 μM MG132 had no little to no effect on accumulation of the pathogenic TDP43 form. Taken together, these results suggest that the fusion protein constructs exert their effects via chaperone-mediated autophagy.

### Example 2: AAV Vectors Encoding Fusion Protein Constructs

An exemplary gene therapy vector is constructed by an AAV9 vector bearing a codon-optimized cDNA encoding the fusion protein constructs of Table 6, specifically constructs 2, 4, 6, 7, 17, and 20-31, as well as control construct 1 (DnaJB1 J domain only), GFP (negative control), under the control of a CAG promoter, containing the cytomegalovirus (CMV) early enhancer element and the chicken beta-actin promoter. The cDNA encoding the construct is located downstream of the Kozak sequence and is polyadenylated by the bovine growth hormone polyadenylation (BGHpA) signal. The entire cassette is flanked by two non-coding terminal inverted sequences of AAV-2.

Recombinant AAV vector is prepared using a baculovirus expression system similar to that described above (Urabe et al., 2002, Unzu et al., 2011 (reviewed in Kotin, 2011)). Briefly, three recombinant baculoviruses, one encoding REP for replication and packaging, one encoding CAP-5 for the capsid of AAV9, and one having an expression cassette is used to infect SF9 insect cells. Purification is performed using AVB Sepharose high speed affinity media (GE Healthcare Life Sciences, Piscataway, NJ). Vectors are titrated using QPCR with the primer-probe combination for the transgene and titers are expressed as genomic copies per ml (GC/ml). The titer of the vector is approximately between 8×10¹³ to 2×10¹⁴ GC/ml.

### Example 3: Testing of Expression and Efficacy in a Mouse Model of ALS

Experiments were first conducted in wild type C57BL/6J mice to confirm expression of Construct 3, and to ascertain whether expression has deleterious effects on the animal. 6×10¹⁰ vg AAV rh10 capsids containing either the control or Construct 3 as described above were injected by either intrathecal or intracerebroventricular injection as shown below in Table 16.

TABLE 16
IT and ICV injection of AAV rh10 containing
an expression vector encoding Construct 3.

| | | | | | | Dosing |
|---|---|---|---|---|---|---|
| | | | | Dose | | Frequency |
| Group | N | Sex | Compounds | (vg/mouse) | Dosing Route | and age |
| 1 | 6 | Male | AAV (empty) | 1 × 10¹¹ | intrathecal | 1 × at 5 weeks |
| 2 | 6 | Male | AAV (Construct-3) | 1 × 10¹¹ | intrathecal | 1 × at 5 weeks |
| 3 | 6 | Male | AAV (empty) | 6 × 10¹⁰ | ICV | 1 × P1 |
| 4 | 6 | Male | AAV (Construct-3) | 6 × 10¹⁰ | ICV | 1 × P1 |

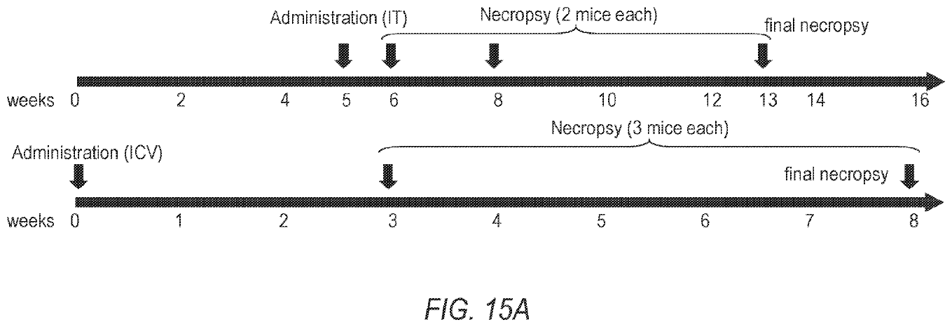

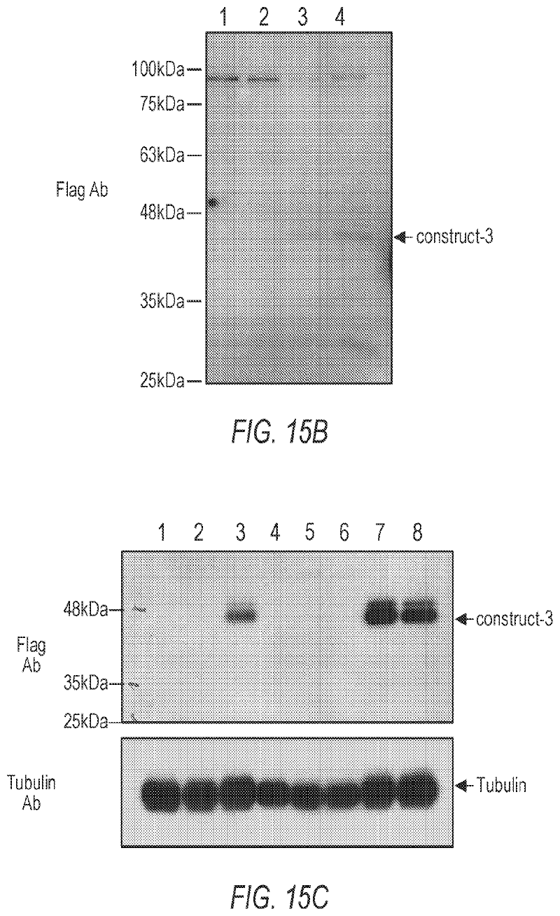

Mice were observed for ataxia, hind limb weakness, or foot dragging. One week after the AAV injection, body weights and clinical observations were made weekly. Mice (n=3) were humanely euthanized by CO2 at 3-week to confirm the AAV expression. No difference in body weight was found during the three weeks after ICV or IT injection (data not shown). As shown in FIG. 15, mice injected by intrathecal (FIG. 15B) or ICV (FIG. 15C) injection resulted in expression of Construct 3, as detected using immunoblots of cerebrum extracts, with no detection in control mice. Furthermore, expression was found to be significantly higher 8 weeks after ICV injection than at 3 weeks.

Vectors prepared above were tested on TDP-43-asociated pathologies of a novel AAV NEFH-tTA×hTDP-43ΔNLS bigenic mice (rNLS mice). This model has a doxycycline (DOX)-repressible construct expressing pathogenic TDP-43 (human TDP-43ΔNLS). Upon removal of DOX, TDP-43ΔNLS expression causes a rapid and progressive deterioration of the animals, causing severe weight loss and, generally within 6-8 weeks, death. We tested the efficacy of Construct 3 (JB1-scFv (3B12A), SEQ ID NO: 80) in slowing the progression of TDP-43ΔNLS-mediated pathology. AAV rh10 containing control or Construct 3 were administered ICV unilaterally at P1/P2 at a volume of 2 ul (maximum 4 ul) in different groups as shown below in Table 17. Except for the control group 1, DOX removal occurred at week 5. Control mice in group

TABLE 17
ICV injection of ΔNLS8 mice

| | | | | | | Time of |
|---|---|---|---|---|---|---|
| Group | No | Mouse Strain | Gender | Treatment | Dose | Administration |
| 1 | 4 | rNLS8 mice (Dox +) | 4Male | AAV (control) | 6 × 10¹⁰ vg | P1 |
| 2 | 8 | rNLS8 mice (Dox −) | 5M/3F | AAV (control) | 6 × 10¹⁰ vg | P1 |
| 3 | 10 | rNLS8 mice (Dox −) | 4M/6F | AAV (Construct3) | 6 × 10¹⁰ vg | P1 |

Body weight was measured twice weekly after weaning. After completion of testing, samples were collected from all surviving mice. Whole brains were collected and split into 2 hemispheres. One hemisphere were weighed and frozen on dry ice. The second hemisphere were post fixed in 4% PFA for histology assessments.

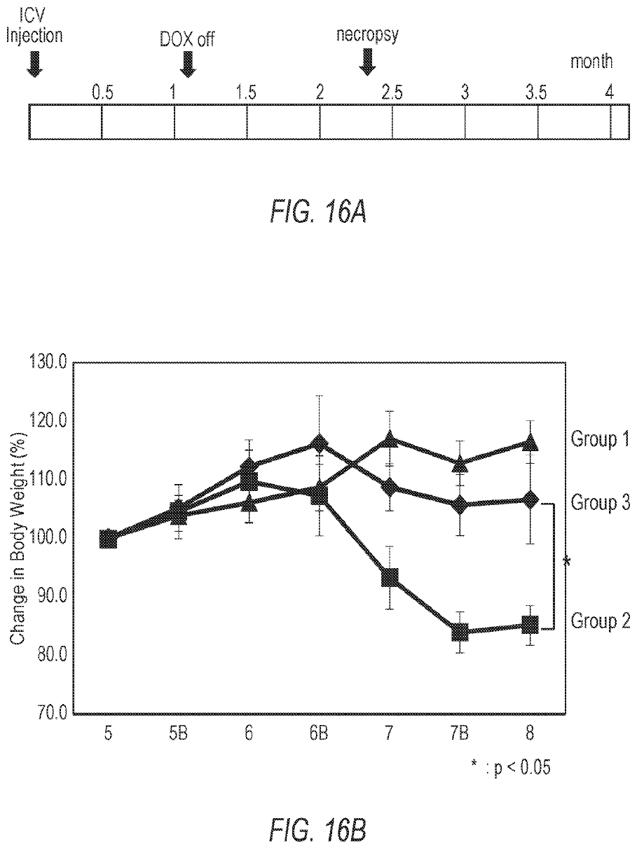

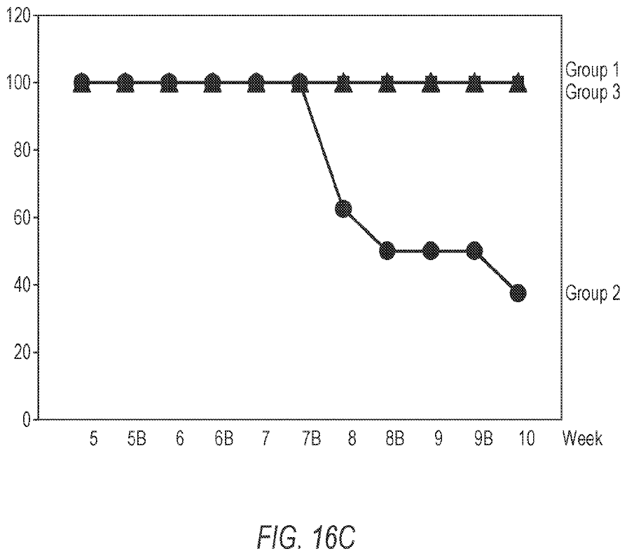

As shown in FIG. 16B, Group 2 control mice, upon DOX removal, began to lose significant weight over the next several weeks, resulting in a statistically significant weight discrepany with Group 1. Surprisingly, Group 3, expressing Construct 3, also exhibited statistically significant weight increase when compared with Group 2 (due to the Group 1 containing only males, results for Group 2 & 3 only show the male body weights to account for gender differences).

When survival was examined, we found that animals in control Group 2 (DOX off) rapidly deteriorated in health, leading to a survival of only 37.5% of the mice at week 10 (0% of the males). However, mice expressing Construct 3 showed 100% survival at week 10, indistinguishable from the Group 1 (DOX on) controls.

### Other Aspects

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific aspects thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and can be applied to the essential features hereinbefore set forth, and follows in the scope of the claimed.

What is claimed is:

1. An isolated fusion protein comprising a J domain of a J protein and a TDP-43-binding domain.

2. (canceled)

3. The fusion protein of claim 1, wherein the J domain of a J protein is of human origin.

4. (canceled)

5. The fusion protein of claim 1, wherein the J domain of a J protein is selected from the group consisting of SEQ ID Nos: 1-50.

6. The fusion protein of claim 5, wherein the J domain comprises the sequence selected from the group consisting of SEQ ID NOs: 1, 5, 6, 10, 16, 24, 25, 31 and 49.

7. The fusion protein of claim 6, wherein the J domain comprises the sequence of SEQ ID NO: 5.

8-12. (canceled)

13. The fusion protein of claim 1, wherein the TDP-43-binding domain comprises the sequence selected from the group consisting of SEQ ID NOs: 51-55.

14. (canceled)

15. The fusion protein of claim 13, wherein the TDP-43-binding domain comprises the sequence of SEQ ID NO:51.

16-19. (canceled)

20. The fusion protein of claim 1, comprising one of the following constructs:
a. DNAJ-X-T,
b. DNAJ-X-T-X-T,
c. DNAJ-X-T-X-T-X-T,
d. T-X-DNAJ,
e. T-X-T-X-DNAJ,
f. T-X-T-X-T-X-DNAJ,
g. T-X-DNAJ-X-T,
h. T-X-DNAJ-X-T-X-T,
i. TDNAJ-X-TTTTTDNAJ-X-T,
j. T-X-T-X-DNAJ-X-TT,
k. TTDNAJ-X-T-X-TTTTTDNAJ-X-T,
l. T-X-T-X-DNAJ-X-T-X-T-X-T,
m. T-X-T-X-T-X-DNAJ-X-T,
n. T-X-T-X-T-X-DNAJ-X-T-X-T,
o. T-X-T-X-T-X-DNAJ-X-T-X-T-X-T,
p. DnaJ-X-DnaJ-X-T-X-T,
q. T-X-DnaJ-X-DnaJ,
r. T-X-T-X-DnaJ-X-DnaJ, and
s. T-X-TDnaJ-X-TDnaJ-X-TTTT
wherein,
T is a TDP-43-binding domain,
DNAJ is a J domain of a J protein, and
X is an optional linker.

21. The fusion protein of claim 20, wherein the fusion protein comprises the J domain sequence of SEQ ID NO: 5 and the TDP-43-binding domain sequence of SEQ ID NO: 51.

22. (canceled)

23. The fusion protein of claim 1, wherein the fusion protein comprises the sequence selected from the group consisting of SEQ ID NOs: 80-85 and 89-97.

24. The fusion protein of claim 23, wherein the fusion protein comprises the sequence selected from the group consisting of SEQ ID NOs: 80, 94, 95 and 96.

25. The fusion protein of claim 23, wherein the fusion protein comprises the sequence of SEQ ID NO: 80.

26. (canceled)

27. (canceled)

28. The fusion protein of claim 23, wherein the fusion protein comprises the sequence of SEQ ID NO: 94.

29. The fusion protein of claim 23, wherein the fusion protein comprises the sequence of SEQ ID NO: 95.

30. The fusion protein of claim 23, wherein the fusion protein comprises the sequence of SEQ ID NO: 96.

31-37. (canceled)

38. The fusion protein of claim 1, which is capable of reducing aggregation of TDP-43 proteins in a cell.

39. The fusion protein of claim 38, which is capable of reducing TDP-43-mediated cytotoxicity.

40. A nucleic acid sequence encoding the fusion protein of any one of claims 1, 3, 5-7, 13, 15, 20, 21, 23-25, 28-30, 38, and 39.

41. The nucleic acid sequence of claim 40, wherein said nucleic acid is DNA.

42. (canceled)

43. (canceled)

44. The nucleic acid sequence of claim 40, further comprising a promoter region, 5′ UTR, and 3′ UTR, such as poly (A) signal.

45. The nucleic acid sequence of claim 44, wherein the promoter region comprises a sequence selected from the group consisting of a CMV enhancer sequence, a CMV promoter, a CBA promoter, UBC promoter, GUSB promoter, NSE promoter, Synapsin promoter, MeCP2 promoter and GFAP promoter.

46. A vector comprising the nucleic acid sequence of any one of claims 40, 41, 44 and 45.

47. The vector of claim 46, wherein the vector is selected from the group consisting of adeno-associated virus (AAV), adenovirus, lentivirus, retrovirus, herpesvirus, poxvirus (vaccinia or myxoma), paramyxovirus (measles, RSV or Newcastle disease virus), baculovirus, reovirus, alphavirus, and flavivirus.

48. The vector of claim 47, wherein the vector is an AAV.

49. A virus particle comprising a capsid and the vector of any one of claims 46-48.

50. The virus particle of claim 49, wherein the capsid is selected from the group consisting of AAV1, AAV2, AAV3, AAV4, AAV5, AAV6, AAV7, AAV8, AAV9, AAV10 AAV11, AAV12, pseudotyped AAV, a rhesus-derived AAV, AAVrh8, AAVrh10 and AAV-DJan AAV capsid mutant, an AAV hybrid serotype, an organ-tropic AAV, a cardiotropic AAV, and a cardiotropic AAVM41 mutant.

51. The virus particle of claim 50, wherein the capsid is selected from the group consisting of AAV2, AAV5, AAV8, AAV9 and AAVrh10.

52-54. (canceled)

55. The virus particle of claim 51, wherein the capsid is AAV9.

56. The virus particle of claim 51, wherein the capsid is AAV rh10.

57. A pharmaceutical composition comprising an agent selected from the group consisting of the fusion protein of any one of claims 1, 3, 5-7, 13, 15, 20, 21, 23-25 28-30, 38, and 39, a cell expressing the fusion protein of claims 1, 3, 5-7, 13, 15, 20, 21 23-25, 28-30, 38, and 39, the nucleic acid of any one of claims 40, 41, 44 and 45, the vector of any one of claims 46-48, the virus particle of any one of claims 49-51, 55 and 56 and a pharmaceutically acceptable carrier or excipient.

58. A method of reducing toxicity of a TDP-43 protein in a cell, comprising contacting said cell with an effective amount of one or more agents selected from the group consisting of the fusion protein of any one of claims 1, 3, 5-7, 13, 15, 20, 21, 23-25, 28-30, 38, and 39, a cell expressing the fusion protein of claims 1, 3, 5-7, 13, 15, 20, 21, 23-25, 28-30, 38, and 39, the nucleic acid of any one of claims 40, 41, 44 and 45, the vector of any one of claims 46-48, the virus particle of any one of claims 49-51, 55 and 56, and the pharmaceutically composition of claim 57.

59. The method of claim 58, wherein the cell is in a subject.

60. The method of claim 59, wherein the subject is a human.

61. The method of claim 59, wherein the cell is a cell of the central nervous system.

62. The method of claim 61, wherein subject is identified as having a TDP-43 disease.

63. The method of claim 62, wherein the TDP-43 disease is selected from the group consisting of ALS, FTD, Parkinson's disease, Huntington's disease, Alzheimer's disease, hippocampal sclerosis, and dementia with Lewy's bodies.

64. The method of claim 63, wherein the TDP-43 disease is ALS.

65. The method of claim 64, wherein there is a reduction in the amount of aggregated TDP-43 protein in the cell when compared with a control cell.

66. A method of treating, preventing, or delaying the progression of a TDP-43 disease in a subject in need thereof, the method comprising administering an effective amount of one or more agents selected from the group consisting of the fusion protein of any one of claims 1, 3, 5-7, 13, 15, 20, 21, 23-25, 28-30, 38, and 39, a cell expressing the fusion protein of claims 1, 3, 5-7, 13, 15, 20, 21, 23-25, 28-30, 38, and 39, the nucleic acid of any one of claims 40, 41, 44 and 45, the vector of any one of claims 46-48, the virus particle of any one of claims 49-51, 55 and 56, and the pharmaceutically composition of claim 57.

67. The method of claim 66, wherein the TDP-43 disease is selected from the group consisting of ALS, FTD, Parkinson's disease, Huntington's disease, Alzheimer's disease, hippocampal sclerosis, and dementia with Lewy's bodies.

68. The method of claim 67, wherein the TDP-43 disease is ALS.

69-71. (canceled)


SEQUENCE LISTING (104 sequences)

SEQ ID NO: 1 - protein, 63 aa, Artificial
  synthetic construct
TYYDVLGVKPNATQEELKKAYRKLALKYHPDKNPNEGEKFKQISQAYEVLSDAKKRELYDKGG

SEQ ID NO: 2 - protein, 63 aa, Artificial
  synthetic construct
KLYDILGVPPGASENELKKAYRKLAKEYHPDKNPNAGDKFKEISFAYEVLSNPEKRELYDRYG

SEQ ID NO: 3 - protein, 66 aa, Artificial
  synthetic construct
DYYQILGVPRNASQKEIKKAYYQLAKKYHPDTNKDDPKAKEKFSQLAEAYEVLSDEVKRKQYDAYG

SEQ ID NO: 4 - protein, 67 aa, Artificial
  synthetic construct
ETQYYDILGVKPSASPEEIKKAYRKLALKYHPDKNPDEGEKFKLISQAYEVLSDPKKRDVYDQGGEQ

SEQ ID NO: 5 - protein, 69 aa, Artificial
  synthetic construct
GKDYYQTLGLARGASDEEIKRAYRRQALRYHPDKNKEPGAEEKFKEIAEAYDVLSDPRKREIFDRYGEE

SEQ ID NO: 6 - protein, 70 aa, Artificial
  synthetic construct
ASYYEILDVPRSASADDIKKAYRRKALQWHPDKNPDNKEFAEKKFKEVAEAYEVLSDKHKREIYDRYGRE

SEQ ID NO: 7 - protein, 69 aa, Artificial
  synthetic construct
MVDYYEVLDVPRQASSEAIKKAYRKLALKWHPDKNPENKEEAERRFKQVAEAYEVLSDAKKRDIYDRYG

SEQ ID NO: 8 - protein, 69 aa, Artificial
  synthetic construct
GKDYYCILGIEKGASDEDIKKAYRKQALKFHPDKNKSPQAEEKFKEVAEAYEVLSDPKKREIYDQFGEE

SEQ ID NO: 9 - protein, 65 aa, Artificial
  synthetic construct
DYYKILGIPSGANEDEIKKAYRKMALKYHPDKNKEPNAEEKFKEIAEAYDVLSDPKKRGLYDQYG

SEQ ID NO: 10 - protein, 68 aa, Artificial
  synthetic construct
VDYYEVLGVQRHASPEDIKKAYRKLALKWHPDKNPENKEEAERKFKQVAEAYEVLSDAKKRDIYDKYG

SEQ ID NO: 11 - protein, 67 aa, Artificial
  synthetic construct
DYYEVLGLQRYASPEDIKKAYHKVALKWHPDKNPENKEEAERKFKEVAEAYEVLSNDEKRDIYDKYG

SEQ ID NO: 12 - protein, 67 aa, Artificial
  synthetic construct
NYYEVLGVQASASPEDIKKAYRKLALRWHPDKNPDNKEEAEKKFKLVSEAYEVLSDSKKRSLYDRAG

SEQ ID NO: 13 - protein, 65 aa, Artificial
  synthetic construct
SYYDILGVPKSASERQIKKAFHKLAMKYHPDKNKSPDAEAKFREIAEAYETLSDANRRKEYDTLG

SEQ ID NO: 14 - protein, 66 aa, Artificial
  synthetic construct
DFYKILGVPRSASIKDIKKAYRKLALQLHPDRNPDDPQAQEKFQDLGAAYEVLSDSEKRKQYDTYG

SEQ ID NO: 15 - protein, 65 aa, Artificial
  synthetic construct
YEILGVSRGASDEDLKKAYRRLALKFHPDKNHAPGATEAFKAIGTAYAVLSNPEKRKQYDQFGDD

SEQ ID NO: 16 - protein, 65 aa, Artificial
  synthetic construct
DYYSVLGITRNSEDAQIKQAYRRLALKHHPLKSNEPSSAEIFRQIAEAYDVLSDPMKRGIYDKFG

SEQ ID NO: 17 - protein, 65 aa, Artificial
  synthetic construct
NYYEVLGVTKDAGDEDLKKAYRKLALKFHPDKNHAPGATDAFKKIGNAYAVLSNPEKRKQYDLTG

SEQ ID NO: 18 - protein, 65 aa, Artificial
  synthetic construct
NFYQFLGVQQDASSADIRKAYRKLSLTLHPDKNKDENAETQFRQLVAIYEVLKDDERRQRYDDIL

SEQ ID NO: 19 - protein, 74 aa, Artificial
  synthetic construct
DHYAVLGLGHVRYKATQRQIKAAHKAMVLKHHPDKRKAAGEPIKEGDNDYFTCITKAYEMLSDPVKRRAFNSVD

SEQ ID NO: 20 - protein, 69 aa, Artificial
  synthetic construct
DYYKILGVKRNAKKQEIIKAYRKLALQWHPDNFQNEEEKKKAEKKFIDIAAAKEVLSDPEMRKKFDDGE

SEQ ID NO: 21 - protein, 66 aa, Artificial
  synthetic construct
TYYELLGVHPGASTEEVKRAFFSKSKELHPDRDPGNPSLHSRFVELSEAYRVLSREQSRRSYDDQL

SEQ ID NO: 22 - protein, 70 aa, Artificial
  synthetic construct
GESLYHVLGLDKNATSDDIKKSYRKLALKYHPDKNPDNPEAADKFKEINNAHAILTDATKRNIYDKYGSL

SEQ ID NO: 23 - protein, 66 aa, Artificial
  synthetic construct
ALYEILGLHKGASNEEIKKTYRKLALKHHPDKNPDDPAATEKFKEINNAHAILTDISKRSIYDKYG

SEQ ID NO: 24 - protein, 65 aa, Artificial
  synthetic construct
TKWKPVGMADLVTPEQVKKVYRKAVLVVHPDKATGQPYEQYAKMIFMELNDAWSEFENQGQKPLY

SEQ ID NO: 25 - protein, 71 aa, Artificial
  synthetic construct
DYYKILGVDKNASEDEIKKAYRKRALMHHPDRHSGASAEVQKEEEKKFKEVGEAFTILSDPKKKTRYDSGQ

SEQ ID NO: 26 - protein, 68 aa, Artificial
  synthetic construct
NPFEVLQIDPEVTDEEIKKRFRQLSILVHPDKNQDDADRAQKAFEAVDKAYKLLLDQEQKKRALDVIQ

SEQ ID NO: 27 - protein, 68 aa, Artificial
  synthetic construct
DLYRVLGVRREASDGEVRRGYHKVSLQVHPDRVGEGDKEDATRRFQILGKVYSVLSDREQRAVYDEQG

SEQ ID NO: 28 - protein, 66 aa, Artificial
  synthetic construct
DFYSLLGVSKTASSREIRQAFKKLALKLHPDKNPNNPNAHGDFLKINRAYEVLKDEDLRKKYDKYG

SEQ ID NO: 29 - protein, 69 aa, Artificial
  synthetic construct
DYYSLLNVRREASSEELKAAYRRLCMLYHPDKHRDPELKSQAERLFNLVHQAYEVLSDPQTRAIYDIYG

SEQ ID NO: 30 - protein, 66 aa, Artificial
  synthetic construct
DYYTLLGCDELSSVEQILAEFKVRALECHPDKHPENPKAVETFQKLQKAKEILTNEESRARYDHWR

SEQ ID NO: 31 - protein, 66 aa, Artificial
  synthetic construct
DAYEVLNLPQGQGPHDESKIRKAYFRLAQKYHPDKNPEGRDMFEKVNKAYEFLCTKSAKIVDGPDP

SEQ ID NO: 32 - protein, 65 aa, Artificial
  synthetic construct
NPFHVLGVEATASDVELKKAYRQLAVMVHPDKNHHPRAEEAFKVLRAAWDIVSNAEKRKEYEMKR

SEQ ID NO: 33 - protein, 55 aa, Artificial
  synthetic construct
EAGLILGVSPSAGKAKIRTAHRRVMILNHPDKGGSPYVAAKINEAKDLLETTTKH

SEQ ID NO: 34 - protein, 65 aa, Artificial
  synthetic construct
DPYRVLGVSRTASQADIKKAYKKLAREWHPDKNKDPGAEDKFIQISKAYEILSNEEKRSNYDQYG

SEQ ID NO: 35 - protein, 66 aa, Artificial
  synthetic construct
DLYALLGIEEKAADKEVKKAYRQKALSCHPDKNPDNPRAAELFHQLSQALEVLTDAAARAAYDKVR

SEQ ID NO: 36 - protein, 65 aa, Artificial
  synthetic construct
NYYEILGVSRDASDEELKKAYRKLALKFHPDKNCAPGATDAFKAIGNAFAVLSNPDKRLRYDEYG

SEQ ID NO: 37 - protein, 55 aa, Artificial
  synthetic construct
EAALILGVSPTANKGKIRDAHRRIMLLNHPDKGGSPYIAAKINEAKDLLEGQAKK

SEQ ID NO: 38 - protein, 72 aa, Artificial
  synthetic construct
DYFSLMDCNRSFRVDTAKLQHRYQQLQRLVHPDFFSQRSQTEKDFSEKHSTLVNDAYKTLLAPLSRGLYLLK

SEQ ID NO: 39 - protein, 67 aa, Artificial
  synthetic construct
CHYEALGVRRDASEEELKKAYRKLALKWHPDKNLDNAAEAAEQFKLIQAAYDVLSDPQERAWYDNHR

SEQ ID NO: 40 - protein, 65 aa, Artificial
  synthetic construct
LAYQVLGLSEGATNEEIHRSYQELVKVWHPDHNLDQTEEAQRHFLEIQAAYEVLSQPRKPWGSRR

SEQ ID NO: 41 - protein, 62 aa, Artificial
  synthetic construct
NPYEVLNLDPGATVAEIKKQYRLLSLKYHPDKGGDEVMFMRIAKAYAALTDEESRKNWEEFG

SEQ ID NO: 42 - protein, 72 aa, Artificial
  synthetic construct
DWYSILGADPSANISDLKQKYQKLILMYHPDKQSTDVPAGTVEECVQKFIEIDQAWKILGNEETKREYDLQR

SEQ ID NO: 43 - protein, 76 aa, Artificial
  synthetic construct
DCYEVLGVSRSAGKAEIARAYRQLARRYHPDRYRPQPGDEGPGRTPQSAEEAFLLVATAYETLKDEETRKDYDYML

SEQ ID NO: 44 - protein, 65 aa, Artificial
  synthetic construct
SRWTPVGMADLVAPEQVKKHYRRAVLAVHPDKAAGQPYEQHAKMIFMELNDAWSEFENQGSRPLF

SEQ ID NO: 45 - protein, 57 aa, Artificial
  synthetic construct
DSWDMLGVKPGASRDEVNKAYRKLAVLLHPDKCVAPGSEDAFKAVVNARTALLKNIK

SEQ ID NO: 46 - protein, 65 aa, Artificial
  synthetic construct
EYYRLLNVEEGCSADEVRESFHKLAKQYHPDSGSNTADSATFIRIEKAYRKVLSHVIEQTNASQS

SEQ ID NO: 47 - protein, 88 aa, Artificial
  synthetic construct
ILKEVTSVVEQAWKLPESERKKIIRRLYLKWHPDKNPENHDIANEVFKHLQNEINRLEKQAFLDQNADRASRRTFSTSASRFQSDKYS

SEQ ID NO: 48 - protein, 66 aa, Artificial
  synthetic construct
ALYDLLGVPSTATQAQIKAAYYRQCFLYHPDRNSGSAEAAERFTRISQAYVVLGSATLRRKYDRGL

SEQ ID NO: 49 - protein, 64 aa, Artificial
  synthetic construct
QLMDLLGLERSAWGNIPLMRKAYLKKCKEFHPDKGGDEEKMKKMNTLYKKMEDGVKYAHQPDFG

SEQ ID NO: 50 - protein, 70 aa, Artificial
  synthetic construct
KQDYYEILGVSKTAEEREIRKAYKRLAMKYHPDRNQGDKEAEAKFKEIKEAYEVLTDSQKRAAYDQYGHA

SEQ ID NO: 51 - protein, 241 aa, Artificial
  synthetic construct
EVQLQQSGAELVKPGASVKLSCTASGFNIKDYYMHWVKQRTEQGLEWIGRIDPEDGETKYAPKFQGKATITADTSSNTAYLQLSSLTSEDTAVYYCTIIYYYGSRYVDYWGQGTTLTVSGGGGSGGGGSGGGGSEIVLTQSPTTMAASPGEKITITCSASSSISSSYLHWYQQKPGFSPKLLIYRTSNLASGVPARFSGSGSGTSYSLTIGTMEAEDVATYYCQQGSSIPLTFGSGTKLEI

SEQ ID NO: 52 - protein, 239 aa, Artificial
  synthetic construct
EVQLVESGGGLVQPGGSLRISCTTSGFIFSDYWMHWVRQAPGKGLTWVSRINLDGSDTIYADSVKGRFTISRDNDKNTLYLQMNSLRVEDTAIYYCARSRKSVWGQGTMVTVSSGGGGSGGGGSGGGGSQSALTQPASVSGSPGQSITISCTGSNTDVGAYDYVSWSQQLPGKAPKFVIFDVDVRPSGISDRFSGSKSGNTASLTISGLQAEDEADYYCSSYTKSGTLVFGGGTKVTVV

SEQ ID NO: 53 - protein, 248 aa, Artificial
  synthetic construct
EVQLLESGGDLVQPGGSLRLSCAASGFTFSSQAMSWVRQAPGKGLEWVSALSRTGDYTWYADSVRGRFTVSRDDSKNIFYLEMNSLRAEDTAVYYCAKNYYSSFGYNWAAFHIWGQGTMVTVSSGGGGSGGGGSGGGGSEIVLTQSPGTLSLSPGERATLSCRASQDVNNNYLAWYQQKPGQAPRLLIYGASRRATGVPDRFSGRGSGTDFTLTINRLEPEDFAMYFCQQYGGSPPYTFGQGTKLEIK

SEQ ID NO: 54 - protein, 25 aa, Artificial
  synthetic construct
QQQQQQQQQQQQQQQQQQQQQQQQQ

SEQ ID NO: 55 - protein, 11 aa, Artificial
  synthetic construct
SNWKWWPGIFD

SEQ ID NO: 56 - skipped

SEQ ID NO: 57 - protein, 4 aa, Artificial
  synthetic construct
GTGS

SEQ ID NO: 58 - protein, 5 aa, Artificial
  synthetic construct
GLESR

SEQ ID NO: 59 - protein, 4 aa, Artificial
  synthetic construct
GGSG

SEQ ID NO: 60 - protein, 4 aa, Artificial
  synthetic construct
GGGS

SEQ ID NO: 61 - protein, 5 aa, Artificial
  synthetic construct
DIAAA

SEQ ID NO: 62 - protein, 9 aa, Artificial
  synthetic construct
DIAAALESR

SEQ ID NO: 63 - protein, 15 aa, Artificial
  synthetic construct
GGGGSGGGGSGGGGS

SEQ ID NO: 64 - protein, 11 aa, Artificial
  synthetic construct
AEAAAKEAAAK

SEQ ID NO: 65 - protein, 15 aa, Artificial
  synthetic construct
SGGGSGGGGSGGGGS

SEQ ID NO: 66 - protein, 25 aa, Artificial
  synthetic construct
DIGGGGSGGGGSGGGGSGGGGSAAA

SEQ ID NO: 67 - protein, 232 aa, Artificial
  synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 8 aa, Artificial
  synthetic construct
DYKDDDDK

SEQ ID NO: 69 - protein, 6 aa, Artificial
  synthetic construct
HHHHHH

SEQ ID NO: 70 - protein, 10 aa, Artificial
  synthetic construct
EQKLISEEDL

SEQ ID NO: 71 - protein, 9 aa, Artificial
  synthetic construct
YPYDVPDYA

SEQ ID NO: 72 - protein, 14 aa, Artificial
  synthetic construct
GKPIPNPLLGLDST

SEQ ID NO: 73 - protein, 220 aa, Homo sapiens
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSD

SEQ ID NO: 74 - protein, 9 aa, Artificial
  synthetic construct
RKKRRQRRR

SEQ ID NO: 75 - protein, 15 aa, Artificial
  synthetic construct
RQIKWFQNRRMKWKK

SEQ ID NO: 76 - protein, 21 aa, Artificial
  synthetic construct
KETWWETWWTEWSQPKKKRKV

SEQ ID NO: 77 - protein, 17 aa, Artificial
  synthetic construct
CSIPPEVKFNKPFVYLI

SEQ ID NO: 78 - protein, 17 aa, Artificial
  synthetic construct
CSIPPEVKFNKPFVYLI

SEQ ID NO: 79 - protein, 253 aa, Artificial
  synthetic construct
MGTGSEFDIAAAEVQLQQSGAELVKPGASVKLSCTASGFNIKDYYMHWVKQRTEQGLEWIGRIDPEDGETKYAPKFQGKATITADTSSNTAYLQLSSLTSEDTAVYYCTIIYYYGSRYVDYWGQGTTLTVSGGGGSGGGGSGGGGSEIVLTQSPTTMAASPGEKITITCSASSSISSSYLHWYQQKPGFSPKLLIYRTSNLASGVPARFSGSGSGTSYSLTIGTMEAEDVATYYCQQGSSIPLTFGSGTKLEI

SEQ ID NO: 80 - protein, 341 aa, Artificial
  synthetic construct
MGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAAEVQLQQSGAELVKPGASVKLSCTASGFNIKDYYMHWVKQRTEQGLEWIGRIDPEDGETKYAPKFQGKATITADTSSNTAYLQLSSLTSEDTAVYYCTIIYYYGSRYVDYWGQGTTLTVSGGGGSGGGGSGGGGSEIVLTQSPTTMAASPGEKITITCSASSSISSSYLHWYQQKPGFSPKLLIYRTSNLASGVPARFSGSGSGTSYSLTIGTMEAEDVATYYCQQGSSIPLTFGSGTKLEI

SEQ ID NO: 81 - protein, 341 aa, Artificial
  synthetic construct
MGKDYYQTLGLARGASDEEIKRAYRRQALRYHQDKNKEPGAEEKFKEIAEAYDVLSDPRKREIFDRYGEEGLKGSDIGGGGSGGGGSGGGGSGGGGSAAAEVQLQQSGAELVKPGASVKLSCTASGFNIKDYYMHWVKQRTEQGLEWIGRIDPEDGETKYAPKFQGKATITADTSSNTAYLQLSSLTSEDTAVYYCTIIYYYGSRYVDYWGQGTTLTVSGGGGSGGGGSGGGGSEIVLTQSPTTMAASPGEKITITCSASSSISSSYLHWYQQKPGFSPKLLIYRTSNLASGVPARFSGSGSGTSYSLTIGTMEAEDVATYYCQQGSSIPLTFGSGTKLEI

SEQ ID NO: 82 - protein, 323 aa, Artificial
  synthetic construct
MGTEVQLQQSGAELVKPGASVKLSCTASGFNIKDYYMHWVKQRTEQGLEWIGRIDPEDGETKYAPKFQGKATITADTSSNTAYLQLSSLTSEDTAVYYCTIIYYYGSRYVDYWGQGTTLTVSGGGGSGGGGSGGGGSEIVLTQSPTTMAASPGEKITITCSASSSISSSYLHWYQQKPGFSPKLLIYRTSNLASGVPARFSGSGSGTSYSLTIGTMEAEDVATYYCQQGSSIPLTFGSGTKLEIGSEFMGKDYYQTLGLARGASDEEIKRAYRRQALRYHPDKNKEPGAEEKFKEIAEAYDVLSDPRKREIFDRYGEEGLKGS

SEQ ID NO: 83 - protein, 589 aa, Artificial
  synthetic construct
MGTEVQLQQSGAELVKPGASVKLSCTASGFNIKDYYMHWVKQRTEQGLEWIGRIDPEDGETKYAPKFQGKATITADTSSNTAYLQLSSLTSEDTAVYYCTIIYYYGSRYVDYWGQGTTLTVSGGGGSGGGGSGGGGSEIVLTQSPTTMAASPGEKITITCSASSSISSSYLHWYQQKPGFSPKLLIYRTSNLASGVPARFSGSGSGTSYSLTIGTMEAEDVATYYCQQGSSIPLTFGSGTKLEIGSEFMGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAAEVQLQQSGAELVKPGASVKLSCTASGFNIKDYYMHWVKQRTEQGLEWIGRIDPEDGETKYAPKFQGKATITADTSSNTAYLQLSSLTSEDTAVYYCTIIYYYGSRYVDYWGQGTTLTVSGGGGSGGGGSGGGGSEIVLTQSPTTMAASPGEKITITCSASSSISSSYLHWYQQKPGFSPKLLIYRTSNLASGVPARFSGSGSGTSYSLTIGTMEAEDVATYYCQQGSSIPLTFGSGTKLEI

SEQ ID NO: 84 - protein, 342 aa, Artificial
  synthetic construct
MVDYYEVLGVQRHASPEDIKKAYRKLALKWHPDKNPENKEEAERKFKQVAEAYEVLSDAKKRDIYDKYGKEGLNGGDIGGGGSGGGGSGGGGSGGGGSAAAEVQLQQSGAELVKPGASVKLSCTASGFNIKDYYMHWVKQRTEQGLEWIGRIDPEDGETKYAPKFQGKATITADTSSNTAYLQLSSLTSEDTAVYYCTIIYYYGSRYVDYWGQGTTLTVSGGGGSGGGGSGGGGSEIVLTQSPTTMAASPGEKITITCSASSSISSSYLHWYQQKPGFSPKLLIYRTSNLASGVPARFSGSGSGTSYSLTIGTMEAEDVATYYCQQGSSIPLTFGSGTKLEI

SEQ ID NO: 85 - protein, 346 aa, Artificial
  synthetic construct
MTKWKPVGMADLVTPEQVKKVYRKAVLVVHPDKATGQPYEQYAKMIFMELNDAWSEFENQGQKPLYDIYDKYGKEGLNGGDIGGGGSGGGGSGGGGSGGGGSAAAEVQLQQSGAELVKPGASVKLSCTASGFNIKDYYMHWVKQRTEQGLEWIGRIDPEDGETKYAPKFQGKATITADTSSNTAYLQLSSLTSEDTAVYYCTIIYYYGSRYVDYWGQGTTLTVSGGGGSGGGGSGGGGSEIVLTQSPTTMAASPGEKITITCSASSSISSSYLHWYQQKPGFSPKLLIYRTSNLASGVPARFSGSGSGTSYSLTIGTMEAEDVATYYCQQGSSIPLTFGSGTKLEI

SEQ ID NO: 86 - protein, 343 aa, Artificial
  synthetic construct
MGTMGKDYYQTLGLARGASDEEIKRAYRRQALRYHPDKNKEPGAEEKFKEIAEAYDVLSDPRKREIFDRYGEEGLKGSGPSGGSGGGANGTSFSYTFHGDPHAMFAEFFGGRNPFDTFFGQRNGEEGMDIDDPFSGFPMGMGGFTNVNFGRSRSAQEPARKKQDPPVTHDLRVSLEEIYSGCTKKMKISHKRLNPDGKSIRNEDKILTIEVKKGWKEGTKITFPKEGDQTSNNIPADIVFVLKDKPHNIFKRDGSDVIYPARISLREALCGCTVNVPTLDGRTIPVVFKDVIRPGMRRKVPGEGLPLPKTPEKRGDLIIEFEVIFPERIPQTSRTVLEQVLPI

SEQ ID NO: 87 - protein, 641 aa, Artificial
  synthetic construct
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 88 - protein, 814 aa, Artificial
  synthetic construct
MSVVGLDVGSQSCYIAVARAGGIETIANEFSDRCTPSVISFGSKNRTIGVAAKNQQITHANNTVSNFKRFHGRAFNDPFIQKEKENLSYDLVPLKNGGVGIKVMYMGEEHLFSVEQITAMLLTKLKETAENSLKKPVTDCVISVPSFFTDAERRSVLDAAQIVGLNCLRLMNDMTAVALNYGIYKQDLPSLDEKPRIVVFVDMGHSAFQVSACAFNKGKLKVLGTAFDPFLGGKNFDEKLVEHFCAEFKTKYKLDAKSKIRALLRLYQECEKLKKLMSSNSTDLPLNIECFMNDKDVSGKMNRSQFEELCAELLQKIEVPLYSLLEQTHLKVEDVSAVEIVGGATRIPAVKERIAKFFGKDISTTLNADEAVARGCALQCAILSPAFKVREFSVTDAVPFPISLIWNHDSEDTEGVHEVFSRNHAAPFSKVLTFLRRGPFELEAFYSDPQGVPYPEAKIGRFVVQNVSAQKDGEKSRVKVKVRVNTHGIFTISTASMVEKVPTEENEMSSEADMECLNQRPPENPDTDANEKKVDQPPEAKKPKIKVVNVELPIEANLVWQLGKDLLNMYIETEGKMIMQDKLEKERNDAKNAVEEYVYEFRDKLCGPYEKFICEQDHQNFLRLLTETEDWLYEEGEDQAKQAYVDKLEELMKIGTPVKVRFQEAEERPKMFEELGQRLQHYAKIAADFRNKDEKYNHIDESEMKKVEKSVNEVMEWMNNVMNAQAKKSLDQDPVVRAQEIKTKIKELNNTCEPVVTQPKPKIESPKLERTPNGPNIDKKEEDLEDKNNFGAEPPHQNGECYPNEKNSVNMDLD

SEQ ID NO: 89 - protein, 339 aa, Artificial
  synthetic construct
MGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAAEVQLVESGGGLVQPGGSLRISCTTSGFIFSDYWMHWVRQAPGKGLTWVSRINLDGSDTIYADSVKGRFTISRDNDKNTLYLQMNSLRVEDTAIYYCARSRKSVWGQGTMVTVSSGGGGSGGGGSGGGGSQSALTQPASVSGSPGQSITISCTGSNTDVGAYDYVSWSQQLPGKAPKFVIFDVDVRPSGISDRFSGSKSGNTASLTISGLQAEDEADYYCSSYTKSGTLVFGGGTKVTVV

SEQ ID NO: 90 - protein, 348 aa, Artificial
  synthetic construct
MGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAAEVQLLESGGDLVQPGGSLRLSCAASGFTFSSQAMSWVRQAPGKGLEWVSALSRTGDYTWYADSVRGRFTVSRDDSKNIFYLEMNSLRAEDTAVYYCAKNYYSSFGYNWAAFHIWGQGTMVTVSSGGGGSGGGGSGGGGSEIVLTQSPGTLSLSPGERATLSCRASQDVNNNYLAWYQQKPGQAPRLLIYGASRRATGVPDRFSGRGSGTDFTLTINRLEPEDFAMYFCQQYGGSPPYTFGQGTKLEIK

SEQ ID NO: 91 - protein, 135 aa, Artificial
  synthetic construct
MGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAASNWKWWPGIFDGLESNWKWWPGIFDSRDYKDDDDK

SEQ ID NO: 92 - protein, 338 aa, Artificial
  synthetic construct
MDYYKILGVDKNASEDEIKKAYRKRALMHHPDRHSGASAEVQKEEEKKFKEVGEAFTILSDPKKKTRYDSGQDIGGGGSGGGGSGGGGSGGGGSAAAEVQLQQSGAELVKPGASVKLSCTASGFNIKDYYMHWVKQRTEQGLEWIGRIDPEDGETKYAPKFQGKATITADTSSNTAYLQLSSLTSEDTAVYYCTIIYYYGSRYVDYWGQGTTLTVSGGGGSGGGGSGGGGSEIVLTQSPTTMAASPGEKITITCSASSSISSSYLHWYQQKPGFSPKLLIYRTSNLASGVPARFSGSGSGTSYSLTIGTMEAEDVATYYCQQGSSIPLTFGSGTKLEI

SEQ ID NO: 93 - protein, 341 aa, Artificial
  synthetic construct
MGQDYYSVLGITRNSEDAQIKQAYRRLALKHHPLKSNEPSSAEIFRQIAEAYDVLSDPMKRGIYDKFGEEGLKGGDIGGGGSGGGGSGGGGSGGGGSAAAEVQLQQSGAELVKPGASVKLSCTASGFNIKDYYMHWVKQRTEQGLEWIGRIDPEDGETKYAPKFQGKATITADTSSNTAYLQLSSLTSEDTAVYYCTIIYYYGSRYVDYWGQGTTLTVSGGGGSGGGGSGGGGSEIVLTQSPTTMAASPGEKITITCSASSSISSSYLHWYQQKPGFSPKLLIYRTSNLASGVPARFSGSGSGTSYSLTIGTMEAEDVATYYCQQGSSIPLTFGSGTKLEI

SEQ ID NO: 94 - protein, 321 aa, Artificial
  synthetic construct
MGKDYYQTLGLARGASDEEIKRAYRRQALRYHPDKNKEPGAEEKFKEIAEAYDVLSDPRKREIFDRYGEEGLKGSGGGGSEVQLQQSGAELVKPGASVKLSCTASGFNIKDYYMHWVKQRTEQGLEWIGRIDPEDGETKYAPKFQGKATITADTSSNTAYLQLSSLTSEDTAVYYCTIIYYYGSRYVDYWGQGTTLTVSGGGGSGGGGSGGGGSEIVLTQSPTTMAASPGEKITITCSASSSISSSYLHWYQQKPGFSPKLLIYRTSNLASGVPARFSGSGSGTSYSLTIGTMEAEDVATYYCQQGSSIPLTFGSGTKLEI

SEQ ID NO: 95 - protein, 321 aa, Artificial
  synthetic construct
MGKDYYQTLGLARGASDEEIKRAYRRQALRYHPDKNKEPGAEEKFKEIAEAYDVLSDPRKREIFDRYGEEGLKGSEAAAKEVQLQQSGAELVKPGASVKLSCTASGFNIKDYYMHWVKQRTEQGLEWIGRIDPEDGETKYAPKFQGKATITADTSSNTAYLQLSSLTSEDTAVYYCTIIYYYGSRYVDYWGQGTTLTVSGGGGSGGGGSGGGGSEIVLTQSPTTMAASPGEKITITCSASSSISSSYLHWYQQKPGFSPKLLIYRTSNLASGVPARFSGSGSGTSYSLTIGTMEAEDVATYYCQQGSSIPLTFGSGTKLEI

SEQ ID NO: 96 - protein, 316 aa, Artificial
  synthetic construct
MGKDYYQTLGLARGASDEEIKRAYRRQALRYHPDKNKEPGAEEKFKEIAEAYDVLSDPRKREIFDRYGEEGLKGSEVQLQQSGAELVKPGASVKLSCTASGFNIKDYYMHWVKQRTEQGLEWIGRIDPEDGETKYAPKFQGKATITADTSSNTAYLQLSSLTSEDTAVYYCTIIYYYGSRYVDYWGQGTTLTVSGGGGSGGGGSGGGGSEIVLTQSPTTMAASPGEKITITCSASSSISSSYLHWYQQKPGFSPKLLIYRTSNLASGVPARFSGSGSGTSYSLTIGTMEAEDVATYYCQQGSSIPLTFGSGTKLEI

SEQ ID NO: 97 - protein, 497 aa, Artificial
  synthetic construct
MGKDYYQTLGLARGASDEEIKRAYRRQALRYHPDKNKEPGAEEKFKEIAEAYDVLSDPRKREIFDRYGEEGLKGSDIGGGGSGGGGSGGGGSAAAEVQLQQSGAELVKPGASVKLSCTASGFNIKDYYMHWVKQRTEQGLEWIGRIDPEDGETKYAPKFQGKATITADTSSNTAYLQLSSLTSEDTAVYYCTIIYYYGSRYVDYWGQGTTLTVSGGGGSGGGGSGGGGSEIVLTQSPTTMAASPGEKITITCSASSSISSSYLHWYQQKPGFSPKLLIYRTSNLASGVPARFSGSGSGTSYSLTIGTMEAEDVATYYCQQGSSIPLTFGSGTKLEILEGDYKDDDDKGSRFTRRGEDLFMCMDIQLVEALCGFQKPISTLDNRTIVITSHPGQIVKHGDIKCVLNEGMPIYRRPYEKGRLIIEFKVNFPENGFLSPDKLSLLEKLLPERKEVEETDEMDQVELVDFDPNQERRRHYNGEAYEDDEHHPRGGVQCQTS

SEQ ID NO: 98 - protein, 17 aa, Artificial
  synthetic construct
MGVKVLFALICIAVAEA

SEQ ID NO: 99 - protein, 19 aa, Artificial
  synthetic construct
MAPVQLLGLLVLFLPAMRC

SEQ ID NO: 100 - protein, 19 aa, Artificial
  synthetic construct
MAVLGLLFCLVTFPSCVLS

SEQ ID NO: 101 - protein, 660 aa, Artificial
  synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKEFDIAAAMSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 102 - protein, 453 aa, Artificial
  synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKEFDIAAAREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 103 - protein, 414 aa, Artificial
  synthetic construct
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 104 - protein, 414 aa, Artificial [sequence omitted]